SEQUENCE LISTING (102 sequences)

SEQ ID NO: 1 - protein, 446 aa, Artificial Sequence
  HC BHA 10 IgG1s knob
QVQLVQSGAEVKKPGSSVKVSCKASGYTFTTYYLHWVRQAPGQGLEWMGWIYPGNVHAQYNEKFKGRVTITADKSTSTAYMELSSLRSEDTAVYYCARSWEGFPYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  LC BHA10
DIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 446 aa, Artificial Sequence
  HC L19 IgG1s knob
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 446 aa, Artificial Sequence
  HC L19 IgG1s hole
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 5 - protein, 215 aa, Artificial Sequence
  LC L19
EIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 450 aa, Artificial Sequence
  HC B21M (RSV) IgG1s knob
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 450 aa, Artificial Sequence
  HC B21M (RSV) IgG1s hole
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 8 - protein, 218 aa, Artificial Sequence
  LC B21M (RSV)
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 450 aa, Artificial Sequence
  HC CBE11 IgG1s knob
EVQLVESGGGLVKPGGSLRLSCAASGFTFSDYYMYWFRQAPGKGLEWVATISDGGSYTYYPDSVKGRFTISRDNAKNSLYLQMSSLRAEDTAVYYCAREENGNFYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  LC CBE11
DIQMTQSPSSLSASVGDRVTITCKAGQDIKSYLSWYQQKPGKAPKLLIYYATRLADGVPSRFSGSGSGTDYTLTISSLQPEDFATYYCLQHGESPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 446 aa, Artificial Sequence
  HC BHA10 IgG1s
QVQLVQSGAEVKKPGSSVKVSCKASGYTFTTYYLHWVRQAPGQGLEWMGWIYPGNVHAQYNEKFKGRVTITADKSTSTAYMELSSLRSEDTAVYYCARSWEGFPYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 446 aa, Artificial Sequence
  HC L19 IgG1s
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 450 aa, Artificial Sequence
  HC CBE11 IgG1s
EVQLVESGGGLVKPGGSLRLSCAASGFTFSDYYMYWFRQAPGKGLEWVATISDGGSYTYYPDSVKGRFTISRDNAKNSLYLQMSSLRAEDTAVYYCAREENGNFYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 450 aa, Artificial Sequence
  HC B21M (RSV) IgG1s
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 744 aa, Artificial Sequence
  3x hmLIGHT fusion to Fc with IgG1s, knob and pA mut
RRSHEVNPAAHLTGANSSLTGSGGPLLWETQLGLAFLRGLSYHDGALVVTKTGYYYIYSKVQLGGVGCPLGLAGTITHGLYKRTPRYPEELELLVSQQSPCGRATSSSRVWWDSSFLGGVVHLEAGEKVVVRVLGKRLVRLRDGTRSYFGAFMVGGGGSGGGGSGGGGSGGGGSRRSHEVNPAAHLTGANSSLTGSGGPLLWETQLGLAFLRGLSYHDGALVVTKTGYYYIYSKVQLGGVGCPLGLAGTITHGLYKRTPRYPEELELLVSQQSPCGRATSSSRVWWDSSFLGGVVHLEAGEKVVVRVLGKRLVRLRDGTRSYFGAFMVGGGGSGGGGSGGGGSGGGGSRRSHEVNPAAHLTGANSSLTGSGGPLLWETQLGLAFLRGLSYHDGALVVTKTGYYYIYSKVQLGGVGCPLGLAGTITHGLYKRTPRYPEELELLVSQQSPCGRATSSSRVWWDSSFLGGVVHLEAGEKVVVRVLGKRLVRLRDGTRSYFGAFMVGGGGSGGGGSGGGGSDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 16 - protein, 502 aa, Artificial Sequence
  3x hmLIGHT single chain used for fusion
RRSHEVNPAAHLTGANSSLTGSGGPLLWETQLGLAFLRGLSYHDGALVVTKTGYYYIYSKVQLGGVGCPLGLAGTITHGLYKRTPRYPEELELLVSQQSPCGRATSSSRVWWDSSFLGGVVHLEAGEKVVVRVLGKRLVRLRDGTRSYFGAFMVGGGGSGGGGSGGGGSGGGGSRRSHEVNPAAHLTGANSSLTGSGGPLLWETQLGLAFLRGLSYHDGALVVTKTGYYYIYSKVQLGGVGCPLGLAGTITHGLYKRTPRYPEELELLVSQQSPCGRATSSSRVWWDSSFLGGVVHLEAGEKVVVRVLGKRLVRLRDGTRSYFGAFMVGGGGSGGGGSGGGGSGGGGSRRSHEVNPAAHLTGANSSLTGSGGPLLWETQLGLAFLRGLSYHDGALVVTKTGYYYIYSKVQLGGVGCPLGLAGTITHGLYKRTPRYPEELELLVSQQSPCGRATSSSRVWWDSSFLGGVVHLEAGEKVVVRVLGKRLVRLRDGTRSYFGAFMV

SEQ ID NO: 17 - protein, 468 aa, Artificial Sequence
  LTa1b2 used for fusion
KPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 18 - protein, 923 aa, Artificial Sequence
  HC B21M fusion to LTa1b2 IgG1s
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 19 - protein, 923 aa, Artificial Sequence
  HC B21M fusion to LTa1b2, IgG1s, knob, with pA mutations
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 20 - protein, 919 aa, Artificial Sequence
  HC L19 fusion to LTa1b2 IgG1s
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 21 - protein, 919 aa, Artificial Sequence
  HC L19 fusion to LTa1b2, IgG1s, knob, with pA mutations
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 22 - protein, 240 aa, Artificial Sequence
  Stapled scFv BHA10 (VH-VL)
QVQLVQSGAEVKKPGSSVKVSCKASGYTFTTYYLHWVRQAPGCGLEWMGWIYPGNVHAQYNEKFKGRVTITADKSTSTAYMELSSLRSEDTAVYYCARSWEGFPYWGQGTTVTVSSGGGSGGGSGCPPCGGGGDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIK

SEQ ID NO: 23 - protein, 239 aa, Artificial Sequence
  Stapled scFv BHA10 (VL-VH)
DIQMTQSPSSLSASVGDRVTITCKASQNVGINVAWYQQKPGCAPKSLISSASYRYSGVPSRFSGSGSGTDFTLTISSLQPEDFATYFCQQYDTYPFTFGQGTKVEIKGGSGGSGGCPPCGSGGQVQLVQSGAEVKKPGSSVKVSCKASGYTFTTYYLHWVRQAPGQGLEWMGWIYPGNVHAQYNEKFKGRVTITADKSTSTAYMELSSLRSEDTAVYYCARSWEGFPYWGCGTTVTVSS

SEQ ID NO: 24 - protein, 17 aa, Artificial Sequence
  Stapled linker (VH-VL)
GGGSGGGSGCPPCGGGG

SEQ ID NO: 25 - protein, 243 aa, Artificial Sequence
  Disulfide stabilized scFv BHA10 (VH-VL)
QVQLVQSGAEVKKPGSSVKVSCKASGYTFTTYYLHWVRQAPGQCLEWMGWIYPGNVHAQYNEKFKGRVTITADKSTSTAYMELSSLRSEDTAVYYCARSWEGFPYWGQGTTVTVSSGGGGSGGGGSGGGGSGGGGSDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIK

SEQ ID NO: 26 - protein, 715 aa, Artificial Sequence
  HC B21M N-term stapled BHA10 (VH-VL), IgG1s, knob, with pA mutations
QVQLVQSGAEVKKPGSSVKVSCKASGYTFTTYYLHWVRQAPGCGLEWMGWIYPGNVHAQYNEKFKGRVTITADKSTSTAYMELSSLRSEDTAVYYCARSWEGFPYWGQGTTVTVSSGGGSGGGSGCPPCGGGGDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIKGGGGSGGGGSGGGGSGGGGSGGGGSQITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 27 - protein, 714 aa, Artificial Sequence
  HCB21M N-term stapled BHA10 (VL-VH), IgG1s, knob, with pA mutations
DIQMTQSPSSLSASVGDRVTITCKASQNVGINVAWYQQKPGCAPKSLISSASYRYSGVPSRFSGSGSGTDFTLTISSLQPEDFATYFCQQYDTYPFTFGQGTKVEIKGGSGGSGGCPPCGSGGQVQLVQSGAEVKKPGSSVKVSCKASGYTFTTYYLHWVRQAPGQGLEWMGWIYPGNVHAQYNEKFKGRVTITADKSTSTAYMELSSLRSEDTAVYYCARSWEGFPYWGCGTTVTVSSGGGGSGGGGSGGGGSGGGGSGGGGSQITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 28 - protein, 705 aa, Artificial Sequence
  HC B21M C-term stapled BHA (VH-VL), IgG1s, knob, with pA mutations
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKGGGGSGGGGSGGGGSQVQLVQSGAEVKKPGSSVKVSCKASGYTFTTYYLHWVRQAPGCGLEWMGWIYPGNVHAQYNEKFKGRVTITADKSTSTAYMELSSLRSEDTAVYYCARSWEGFPYWGQGTTVTVSSGGGSGGGSGCPPCGGGGDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIK

SEQ ID NO: 29 - protein, 704 aa, Artificial Sequence
  HC B21M C-term stapled BHA (VL-VH), IgG1s, knob, with pA mutations
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKGGGGSGGGGSGGGGSDIQMTQSPSSLSASVGDRVTITCKASQNVGINVAWYQQKPGCAPKSLISSASYRYSGVPSRFSGSGSGTDFTLTISSLQPEDFATYFCQQYDTYPFTFGQGTKVEIKGGSGGSGGCPPCGSGGQVQLVQSGAEVKKPGSSVKVSCKASGYTFTTYYLHWVRQAPGQGLEWMGWIYPGNVHAQYNEKFKGRVTITADKSTSTAYMELSSLRSEDTAVYYCARSWEGFPYWGCGTTVTVSS

SEQ ID NO: 30 - protein, 711 aa, Artificial Sequence
  HC L19 N-term stapled BHA10 (VH-VL), IgG1s, knob, with pA mutations
QVQLVQSGAEVKKPGSSVKVSCKASGYTFTTYYLHWVRQAPGCGLEWMGWIYPGNVHAQYNEKFKGRVTITADKSTSTAYMELSSLRSEDTAVYYCARSWEGFPYWGQGTTVTVSSGGGSGGGSGCPPCGGGGDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIKGGGGSGGGGSGGGGSGGGGSGGGGSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 31 - protein, 710 aa, Artificial Sequence
  HC L19 N-term stapled BHA10 (VL-VH), IgG1s, knob, with pA mutations
DIQMTQSPSSLSASVGDRVTITCKASQNVGINVAWYQQKPGCAPKSLISSASYRYSGVPSRFSGSGSGTDFTLTISSLQPEDFATYFCQQYDTYPFTFGQGTKVEIKGGSGGSGGCPPCGSGGQVQLVQSGAEVKKPGSSVKVSCKASGYTFTTYYLHWVRQAPGQGLEWMGWIYPGNVHAQYNEKFKGRVTITADKSTSTAYMELSSLRSEDTAVYYCARSWEGFPYWGCGTTVTVSSGGGGSGGGGSGGGGSGGGGSGGGGSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 32 - protein, 701 aa, Artificial Sequence
  HC L19 C-term stapled BHA10 (VH-VL), IgG1s, knob, with pA mutations
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKGGGGSGGGGSGGGGSQVQLVQSGAEVKKPGSSVKVSCKASGYTFTTYYLHWVRQAPGCGLEWMGWIYPGNVHAQYNEKFKGRVTITADKSTSTAYMELSSLRSEDTAVYYCARSWEGFPYWGQGTTVTVSSGGGSGGGSGCPPCGGGGDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIK

SEQ ID NO: 33 - protein, 700 aa, Artificial Sequence
  HC L19 C-term stapled BHA10 (VL-VH), IgG1s, knob, with pA mutations
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKGGGGSGGGGSGGGGSDIQMTQSPSSLSASVGDRVTITCKASQNVGINVAWYQQKPGCAPKSLISSASYRYSGVPSRFSGSGSGTDFTLTISSLQPEDFATYFCQQYDTYPFTFGQGTKVEIKGGSGGSGGCPPCGSGGQVQLVQSGAEVKKPGSSVKVSCKASGYTFTTYYLHWVRQAPGQGLEWMGWIYPGNVHAQYNEKFKGRVTITADKSTSTAYMELSSLRSEDTAVYYCARSWEGFPYWGCGTTVTVSS

SEQ ID NO: 34 - protein, 701 aa, Artificial Sequence
  HC L19 C-term stapled (VL3_Y36F_S49Y_F87Y) BHA (VH-VL), IgG1s, knob, with pA mutations
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKGGGGSGGGGSGGGGSQVQLVQSGAEVKKPGSSVKVSCKASGYTFTTYYLHWVRQAPGCGLEWMGWIYPGNVHAQYNEKFKGRVTITADKSTSTAYMELSSLRSEDTAVYYCARSWEGFPYWGQGTTVTVSSGGGSGGGSGCPPCGGGGDIQMTQSPSSLSASVGDRVTITCKASQNVGINVAWFQQKPGKAPKSLIYSASYRYSGVPSRFSGSGSGTDFTLTISSLQPEDFATYYCQQYDTYPFTFGCGTKVEIK

SEQ ID NO: 35 - protein, 701 aa, Artificial Sequence
  HC L19 C-term stapled (VH_CDR1_Y33A) BHA10 (VH-VL), IgG1s, knob, with pA mutations
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKGGGGSGGGGSGGGGSQVQLVQSGAEVKKPGSSVKVSCKASGYTFTTYALHWVRQAPGCGLEWMGWIYPGNVHAQYNEKFKGRVTITADKSTSTAYMELSSLRSEDTAVYYCARSWEGFPYWGQGTTVTVSSGGGSGGGSGCPPCGGGGDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIK

SEQ ID NO: 36 - protein, 705 aa, Artificial Sequence
  HC B21M C-term stapled (VL3_Y36F_S49Y_F87Y) BHA (VH-VL), IgG1s, knob, with pA mutations
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKGGGGSGGGGSGGGGSQVQLVQSGAEVKKPGSSVKVSCKASGYTFTTYYLHWVRQAPGCGLEWMGWIYPGNVHAQYNEKFKGRVTITADKSTSTAYMELSSLRSEDTAVYYCARSWEGFPYWGQGTTVTVSSGGGSGGGSGCPPCGGGGDIQMTQSPSSLSASVGDRVTITCKASQNVGINVAWFQQKPGKAPKSLIYSASYRYSGVPSRFSGSGSGTDFTLTISSLQPEDFATYYCQQYDTYPFTFGCGTKVEIK

SEQ ID NO: 37 - protein, 705 aa, Artificial Sequence
  HC B21M C-term stapled (VH_CDR1_Y33A) BHA10 (VH-VL), IgG1s, knob, with pA mutations
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKGGGGSGGGGSGGGGSQVQLVQSGAEVKKPGSSVKVSCKASGYTFTTYALHWVRQAPGCGLEWMGWIYPGNVHAQYNEKFKGRVTITADKSTSTAYMELSSLRSEDTAVYYCARSWEGFPYWGQGTTVTVSSGGGSGGGSGCPPCGGGGDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIK

SEQ ID NO: 38 - protein, 701 aa, Artificial Sequence
  HC L19 C-term stapled BHA10 (VH-VL), IgG1s, knob, no pA mutations
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGSSVKVSCKASGYTFTTYYLHWVRQAPGCGLEWMGWIYPGNVHAQYNEKFKGRVTITADKSTSTAYMELSSLRSEDTAVYYCARSWEGFPYWGQGTTVTVSSGGGSGGGSGCPPCGGGGDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIK

SEQ ID NO: 39 - protein, 704 aa, Artificial Sequence
  HC L19 C-term disulfide stab, BHA (VH-VL), IgG1s, knob, without pA mutations
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGSSVKVSCKASGYTFTTYYLHWVRQAPGQCLEWMGWIYPGNVHAQYNEKFKGRVTITADKSTSTAYMELSSLRSEDTAVYYCARSWEGFPYWGQGTTVTVSSGGGGSGGGGSGGGGSGGGGSDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIK

SEQ ID NO: 40 - protein, 708 aa, Artificial Sequence
  HC B21M C-term disulfide stab, BHA10 (VH-VL), IgG1s, knob, without pA mutations
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGSSVKVSCKASGYTFTTYYLHWVRQAPGQCLEWMGWIYPGNVHAQYNEKFKGRVTITADKSTSTAYMELSSLRSEDTAVYYCARSWEGFPYWGQGTTVTVSSGGGGSGGGGSGGGGSGGGGSDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIK

SEQ ID NO: 41 - protein, 705 aa, Artificial Sequence
  HC B21M C-term stapled BHA (VH-VL), IgG1s, knob, no pA mutations
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGSSVKVSCKASGYTFTTYYLHWVRQAPGCGLEWMGWIYPGNVHAQYNEKFKGRVTITADKSTSTAYMELSSLRSEDTAVYYCARSWEGFPYWGQGTTVTVSSGGGSGGGSGCPPCGGGGDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIK

SEQ ID NO: 42 - protein, 20 aa, Artificial Sequence
  (GGGGS)4 linker
GGGGSGGGGSGGGGSGGGGS

SEQ ID NO: 43 - protein, 116 aa, Artificial Sequence
  VH BHA10
QVQLVQSGAEVKKPGSSVKVSCKASGYTFTTYYLHWVRQAPGQGLEWMGWIYPGNVHAQYNEKFKGRVTITADKSTSTAYMELSSLRSEDTAVYYCARSWEGFPYWGQGTTVTVSS

SEQ ID NO: 44 - protein, 107 aa, Artificial Sequence
  VL BHA10
DIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGQGTKVEIK

SEQ ID NO: 45 - protein, 116 aa, Artificial Sequence [sequence omitted]

SEQ ID NO: 46 - protein, 108 aa, Artificial Sequence [sequence omitted]

SEQ ID NO: 47 - protein, 120 aa, Artificial Sequence
  VH CBE11
EVQLVESGGGLVKPGGSLRLSCAASGFTFSDYYMYWFRQAPGKGLEWVATISDGGSYTYYPDSVKGRFTISRDNAKNSLYLQMSSLRAEDTAVYYCAREENGNFYYFDYWGQGTTVTVSS

SEQ ID NO: 48 - protein, 107 aa, Artificial Sequence
  VL CBE11
DIQMTQSPSSLSASVGDRVTITCKAGQDIKSYLSWYQQKPGKAPKLLIYYATRLADGVPSRFSGSGSGTDYTLTISSLQPEDFATYYCLQHGESPWTFGGGTKLEIK

SEQ ID NO: 49 - protein, 120 aa, Artificial Sequence
  VH B21M
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSS

SEQ ID NO: 50 - protein, 111 aa, Artificial Sequence
  VL B21M
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIK

SEQ ID NO: 51 - protein, 371 aa, Artificial Sequence
  Fibronectin domains 7B89
PLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTHHHHHH

SEQ ID NO: 52 - protein, 280 aa, Artificial Sequence
  Fibronectin domains 789
PLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTHHHHHH

SEQ ID NO: 53 - protein, 240 aa, Artificial Sequence
  Stapled scFv (VL3_Y36F_S49Y_F87Y) BHA10 (VH-VL)
QVQLVQSGAEVKKPGSSVKVSCKASGYTFTTYYLHWVRQAPGCGLEWMGWIYPGNVHAQYNEKFKGRVTITADKSTSTAYMELSSLRSEDTAVYYCARSWEGFPYWGQGTTVTVSSGGGSGGGSGCPPCGGGGDIQMTQSPSSLSASVGDRVTITCKASQNVGINVAWFQQKPGKAPKSLIYSASYRYSGVPSRFSGSGSGTDFTLTISSLQPEDFATYYCQQYDTYPFTFGCGTKVEIK

SEQ ID NO: 54 - protein, 240 aa, Artificial Sequence
  Stapled scFv (VH_CDR1_Y33A) BHA10 (VH-VL)
QVQLVQSGAEVKKPGSSVKVSCKASGYTFTTYALHWVRQAPGCGLEWMGWIYPGNVHAQYNEKFKGRVTITADKSTSTAYMELSSLRSEDTAVYYCARSWEGFPYWGQGTTVTVSSGGGSGGGSGCPPCGGGGDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIK

SEQ ID NO: 55 - protein, 16 aa, Artificial Sequence
  Stapled linker (VL-VH)
GGSGGSGGCPPCGSGG

SEQ ID NO: 56 - protein, 704 aa, Artificial Sequence
  HC L19 C-term disulfide stab BHA10 (VH-VL), IgG1s, knob, with pA mutations
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKGGGGSGGGGSGGGGSQVQLVQSGAEVKKPGSSVKVSCKASGYTFTTYYLHWVRQAPGQCLEWMGWIYPGNVHAQYNEKFKGRVTITADKSTSTAYMELSSLRSEDTAVYYCARSWEGFPYWGQGTTVTVSSGGGGSGGGGSGGGGSGGGGSDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIK

SEQ ID NO: 57 - protein, 708 aa, Artificial Sequence
  HC B21M C-term disulfide stab, BHA10 (VH-VL), IgG1s, knob, with pA mutations
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKGGGGSGGGGSGGGGSQVQLVQSGAEVKKPGSSVKVSCKASGYTFTTYYLHWVRQAPGQCLEWMGWIYPGNVHAQYNEKFKGRVTITADKSTSTAYMELSSLRSEDTAVYYCARSWEGFPYWGQGTTVTVSSGGGGSGGGGSGGGGSGGGGSDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIK

SEQ ID NO: 58 - protein, 227 aa, Artificial Sequence
  IgG1 sigma Fc
DKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 15 aa, Artificial Sequence
  (GGGGS)3 linker
GGGGSGGGGSGGGGS

SEQ ID NO: 60 - protein, 5 aa, Artificial Sequence
  BHA10 HCDR1
TYYLH

SEQ ID NO: 61 - protein, 17 aa, Artificial Sequence
  BHA10 HCDR2
WIYPGNVHAQYNEKFKG

SEQ ID NO: 62 - protein, 7 aa, Artificial Sequence
  BHA10 HCDR3
SWEGFPY

SEQ ID NO: 63 - protein, 11 aa, Artificial Sequence
  BHA10 LCDR1
KASQNVGINVA

SEQ ID NO: 64 - protein, 7 aa, Artificial Sequence
  BHA10 LCDR2
SASYRYS

SEQ ID NO: 65 - protein, 9 aa, Artificial Sequence
  BHA10 LCDR3
QQYDTYPFT

SEQ ID NO: 66 - protein, 5 aa, Artificial Sequence
  CBE11 HCDR1
DYYMY

SEQ ID NO: 67 - protein, 16 aa, Artificial Sequence
  CBE11 HCDR2
TISDGGSYTYYPDSVK

SEQ ID NO: 68 - protein, 11 aa, Artificial Sequence
  CBE11 HCDR3
EENGNFYYFDY

SEQ ID NO: 69 - protein, 11 aa, Artificial Sequence
  CBE11 LCDR1
KAGQDIKSYLS

SEQ ID NO: 70 - protein, 7 aa, Artificial Sequence
  CBE11 LCDR2
YATRLAD

SEQ ID NO: 71 - protein, 9 aa, Artificial Sequence
  CBE11 LCDR3
LQHGESPWT

SEQ ID NO: 72 - protein, 5 aa, Artificial Sequence
  L19 HCDR1
SFSMS

SEQ ID NO: 73 - protein, 17 aa, Artificial Sequence
  L19 HCDR2
SISGSSGTTYYADSVKG

SEQ ID NO: 74 - protein, 7 aa, Artificial Sequence
  L19 HCDR3
PFPYFDY

SEQ ID NO: 75 - protein, 12 aa, Artificial Sequence
  L19 LCDR1
RASQSVSSSFLA

SEQ ID NO: 76 - protein, 7 aa, Artificial Sequence
  L19 LCDR2
YASSRAT

SEQ ID NO: 77 - protein, 9 aa, Artificial Sequence
  L19 LCDR3
QQTGRIPPT

SEQ ID NO: 78 - protein, 119 aa, Artificial Sequence
  VH MSLN mAb1
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSS

SEQ ID NO: 79 - protein, 106 aa, Artificial Sequence
  VL MSLN mAb1
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 80 - protein, 704 aa, Artificial Sequence
  MSLN mAb1 HC C-term stapled BHA10 (VH-VL), IgG1s, knob, with pA mutations
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGKGGGGSGGGGSGGGGSQVQLVQSGAEVKKPGSSVKVSCKASGYTFTTYYLHWVRQAPGCGLEWMGWIYPGNVHAQYNEKFKGRVTITADKSTSTAYMELSSLRSEDTAVYYCARSWEGFPYWGQGTTVTVSSGGGSGGGSGCPPCGGGGDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIK

SEQ ID NO: 81 - protein, 449 aa, Artificial Sequence
  HC MSLN mAb1 IgG1s hole
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 82 - protein, 213 aa, Artificial Sequence
  LC MSLN mAb1
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 5 aa, Artificial Sequence
  BHA10 HCDR1 low affinity variant
TYALH

SEQ ID NO: 84 - protein, 445 aa, Artificial Sequence
  HC L19 IgG1s knob with pA mutations
VQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 85 - protein, 450 aa, Artificial Sequence
  HC B21M (RSV) IgG1s knob with pA mutations
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 86 - protein, 449 aa, Artificial Sequence
  MSLNmAb1 HC, IgG1s, knob, with pA mutations
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 87 - protein, 121 aa, Artificial Sequence
  VH of Anti-TnCA2 Ab 2B10
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARLYGYAYYGAFDYWGQGTTVTVSS

SEQ ID NO: 88 - protein, 107 aa, Artificial Sequence
  VL of Anti-TnCA2 Ab 2B10
DIQMTQSPSSLSASVGDRVTITCRASQGIRNDLGWYQQKPGKAPKRLIYAASSLQSGVPSRFSGGGSGTEFTLTISSLQPEDFATYYCLQNGLQPATFGQGTKVEIK

SEQ ID NO: 89 - protein, 118 aa, Artificial Sequence
  VH Anti-EDA Ab F8
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 90 - protein, 108 aa, Artificial Sequence
  VL Anti-EDA Ab F8
EIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 91 - protein, 278 aa, Artificial Sequence
  Hu Fibronectin domains 11-A-12
EIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLEHHHHHH

SEQ ID NO: 92 - protein, 188 aa, Artificial Sequence
  Hu Fibronectin domains 11-12
EIDKPSQMQVTDVQDNSISVKWLPSSSPVTGYRVTTTPKNGPGPTKTKTAGPDQTEMTIEGLQPTVEYVVSVYAQNPSGESQPLVQTAVTTIPAPTDLKFTQVTPTSLSAQWTPPNVQLTGYRVRVTPKEKTGPMKEINLAPDSSSVVVSGLMVATKYEVSVYALKDTLTSRPAQGVVTTLEHHHHHH

SEQ ID NO: 93 - protein, 279 aa, Artificial Sequence
  Hu Tenascin C domains A1-A2-A3
EQAPELENLTVTEVGWDGLRLNWTAADQAYEHFIIQVQEANKVEAARNLTVPGSLRAVDIPGLKAATPYTVSIYGVIQGYRTPVLSAEASTGETPNLGEVVVAEVGWDALKLNWTAPEGAYEYFFIQVQEADTVEAAQNLTVPGGLRSTDLPGLKAATHYTITIRGVTQDFSTTPLSVEVLTEEVPDMGNLTVTEVSWDALRLNWTTPDGTYDQFTIQVQEADQVEEAHNLTVPGSLRSMEIPGLRAGTPYTVTLHGEVRGHSTRPLAVEVVTHHHHHH

SEQ ID NO: 94 - protein, 185 aa, Artificial Sequence
  Hu Tenascin C domains A1-A3
EQAPELENLTVTEVGWDGLRLNWTAADQAYEHFIIQVQEANKVEAARNLTVPGSLRAVDIPGLKAATPYTVSIYGVIQGYRTPVLSAEEEVPDMGNLTVTEVSWDALRLNWTTPDGTYDQFTIQVQEADQVEEAHNLTVPGSLRSMEIPGLRAGTPYTVTLHGEVRGHSTRPLAVEVVTHHHHHH

SEQ ID NO: 95 - protein, 451 aa, Artificial Sequence
  2B10 HC, IgG1 sigma and hole mutations
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARLYGYAYYGAFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 96 - protein, 706 aa, Artificial Sequence
  2B10 HC with BHA10 stapled (VH-VL) scFv C-ter fused, IgG1 sigma, knob mutations
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARLYGYAYYGAFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGSSVKVSCKASGYTFTTYYLHWVRQAPGCGLEWMGWIYPGNVHAQYNEKFKGRVTITADKSTSTAYMELSSLRSEDTAVYYCARSWEGFPYWGQGTTVTVSSGGGSGGGSGCPPCGGGGDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIK

SEQ ID NO: 97 - protein, 214 aa, Artificial Sequence
  2B10 LC
DIQMTQSPSSLSASVGDRVTITCRASQGIRNDLGWYQQKPGKAPKRLIYAASSLQSGVPSRFSGGGSGTEFTLTISSLQPEDFATYYCLQNGLQPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 448 aa, Artificial Sequence
  F8 HC, IgG1 sigma and hole mutations
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 99 - protein, 703 aa, Artificial Sequence
  F8 HC with BHA10 stapled (VH-VL) scFv C-ter fused, IgG1 sigma, knob mutations
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGSSVKVSCKASGYTFTTYYLHWVRQAPGCGLEWMGWIYPGNVHAQYNEKFKGRVTITADKSTSTAYMELSSLRSEDTAVYYCARSWEGFPYWGQGTTVTVSSGGGSGGGSGCPPCGGGGDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIK

SEQ ID NO: 100 - protein, 215 aa, Artificial Sequence
  F8 LC
EIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 448 aa, Artificial Sequence
  F8 HC, IgG1 sigma, knob mutations
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 451 aa, Artificial Sequence
  2B10 HC, IgG1 sigma, knob mutations
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARLYGYAYYGAFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGASSVFLFPPKPKDTLMISRTPEVTCVVVDVSAEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2021/0188990 A1
(43) Pub. Date: Jun. 24, 2021

(54) MULTISPECIFIC BINDING MOLECULES COMPRISING LTBR AND EDB BINDING DOMAINS AND USES THEREOF

(71) Applicant: Cilag GmbH International, Zug (CH)

(72) Inventors: Matthew LORENZI, Philadelphia, PA (US); Sylvie LAQUERRE, Wayne, PA (US); Simon BRACK, Winterthur (CH); Kristina KLUPSCH, Zurich (CH); Babette SCHADE, Zurich-Schlieren (CH); Vanessa BAERISWYL, Zurich (CH); Michela SILACCI MELKKO, Zurich (CH); Julian BERTSCHINGER, Zurich (CH)

(21) Appl. No.: 17/117,304

(22) Filed: Dec. 10, 2020

Related U.S. Application Data
(60) Provisional application No. 62/946,452, filed on Dec. 11, 2019.

(51) Int. Cl. C07K 16/28; A61K 45/06

(52) U.S. Cl. CPC C07K 16/2878; A61K 2039/505; C07K 2317/31; A61K 45/06

(57) ABSTRACT

Provided herein are anti-LTBR multispecific binding molecules, nucleic acids encoding the anti-LTBR multispecific binding molecules, vectors comprising the nucleic acids, host cells comprising the vectors, and pharmaceutical compositions comprising the anti-LTBR multispecific binding molecules. Also provided are methods of treating cancer in a subject in need thereof, the methods comprising administering the pharmaceutical compositions disclosed herein.

### CROSS-REFERENCE TO RELATED APPLICATIONS

This application claims the benefit of U.S. Provisional Application Ser. No. 62/946,452, filed 11 Dec. 2019. The entire content of the aforementioned application is incorporated herein by reference in its entirety.

### FIELD OF THE INVENTION

This invention relates to anti-LTBR multispecific binding molecules, nucleic acids and expression vectors encoding the binding molecules, recombinant cells containing the vectors, and compositions comprising the binding molecules. Methods of making the binding molecules, and methods of using the binding molecules to kill cancer cells, are also provided.

### BACKGROUND OF THE INVENTION

Immunotherapy of cancer has the potential to improve the survival of cancer patients by promoting an immune response towards the tumor. While certain patients experience deep and long responses to currently available anti-cancer immunotherapy (e.g. anti-CTLA4 antibody ipilimumab, anti-PD-1/PD-L1 antibodies such as pembrolizumab or nivolumab), a large fraction of patients do not benefit from the such therapies (Ribas et al., Science 359:1350-1355 (2018)). For instance, patients that have so called “cold” or non-inflamed tumors that are characterized by the lack of immune cell infiltrate or by the absence of an inflammatory signature have a lower benefit from anti-cancer immunotherapies (Chen and Mellman, Nature 541:321-30 (2017)). Thus, there is a need for novel anti-cancer immunotherapies with improved efficacy.

The lymphotoxin beta receptor (LTBR/TNFRSF3), a receptor of the TNF superfamily, is one of many possible targets for anti-cancer immunotherapy. LTBR plays a central role in the development and homeostasis of lymph nodes and secondary lymphoid organs by regulating the expression of several homeostatic lymphoid cytokines (e.g. CCL19, CCL21, CXCL13) and adhesion molecules (ICAM-1, VCAM-1, MAdCAM1) via the NF-κB pathway (Dejardin et al., Immunity 17:525-535 (2002), Schneider et al., Immunol. Rev. 202:49-66 (2004)). LTBR is activated by two different trimeric ligands, LIGHT (TNFSF14) and lymphotoxin α1β2 (LTα1β2). Whereas LTα1β2 is specific for LTBR, LIGHT also binds to and activates HVEM (TNFRSF14), a receptor expressed on and implicated in the regulation of immune cells (Pasero et al., Curr. Opin. Pharmacol. 12:478-85 (2012)).

Of particular interest from a cancer immunotherapy perspective is the finding that activation of LTBR by its ligands leads to ectopic formation of tertiary lymphoid structures (TLS) (Schrama et al., Immunity 14:111-121 (2001); Tang et al., Cell. Mol. Immunol. 14:809-18 (2017)). Presence of TLS in the tumor microenvironment typically correlates with immune infiltration and is also associated with better prognosis, suggesting that TLS are involved in anti-tumor immune responses (Dieu-Nosjean et al., J. Clin. Oncol. 26:4410-17 (2008; Weinstein and Storkus, Adv. Cancer Res. 128:197-233 (2015)). Therefore, activation of LTBR has the potential to promote TLS formation in the tumor microenvironment and induce anti-tumor immune responses and improve current cancer immunotherapies.

The therapeutic concept of targeting LTBR with the aim to promote a protective anti-tumor immune response has been established in several preclinical studies.

Several groups have targeted LTBR using its natural ligand LIGHT (TNFSF14). LIGHT binds to LTBR as well as to a second receptor, HVEM (TNFRSF14), which is expressed on immune cells such as B cells, T cells, NK cells, monocytes and DCs (Pasero et al., Curr. Opin. Pharmacol. 12:478-85 (2012)). Therefore, it should be noted that LIGHT-mediated immune biological effects might depend either on LTBR or on HVEM.

Yu et al. demonstrated that forced expression of a membrane-bound form of LIGHT in murine tumor cell lines resulted in massive infiltration of naive T lymphocytes that correlated with an upregulation of chemokine production and adhesion molecules, resulting in rejection of established tumors at local and distal sites (Yu et al., Nat. Immunol. 5:141-9 (2004)). Similar findings were made in a setting when forced expression of membrane-bound LIGHT was achieved through adenoviral delivery of the LIGHT gene into established tumors (Yu et al., J. Immunol. 179:1960-8 (2007)).

Building on these findings, and in an attempt to exploit this mode-of-action with a modality that is better suitable for clinical applications, Tang and colleagues generated a homotrimeric single-chain LIGHT variant with improved stability and with human and mouse cross-reactivity, termed 3×hmLIGHT (Tang et al., Cancer Cell 29:285-96 (2016)). When fused to an EGFR-specific tumor targeting antibody, 3×hmLIGHT induced anti-tumor immunity in mouse and human tumor models by increasing lymphocyte infiltration and thereby could overcome resistance to checkpoint blockade immunotherapy when combined with an anti-PD-L1 antibody in models with low lymphocyte infiltration. Tang et al., reported on tolerability upon intratumoral injection into tumor bearing mice. No significant side effects were observed as no significant changes in body weight or serum cytokines were seen. The authors did not report on tolerability after systemic administration.

Johansson-Percival et al. developed a fusion construct composed of mouse LIGHT fused to the C-terminus of a vascular targeting peptide (VTP) (Johansson-Percival et al., Nat. Immunol. 18:1207-17 (2017)). In a murine solid tumor model, the VTP-LIGHT construct homed to tumor vessels, promoted vessel normalization and induced TLS. Addition of VTP-LIGHT enhanced the activity of a combination of anti-CTLA4 and anti-PD-1 antibodies and of anti-tumor vaccination in vivo. Weight loss was observed in treated mice after intravenous administration of VTP-LIGHT.

Gurney et al. reported in vitro and in vivo studies with a bispecific fusion construct consisting of a heterotrimeric single-chain LTα1β2 moiety fused to a B7-H4 specific tumor-targeting antibody (WO2018/119118). Importantly, unlike LIGHT used in the various approaches mentioned above, the LTα1β2 fusion construct is a specific agonist of LTBR and does not activate HVEM. Infiltration of immune cells, induced cytokine expression, and formation of TLS were observed after treatment with the LTα1β2 fusion construct in murine tumor models. The anti-tumor activity of the LTα1β2-antibody fusion combined with an anti-PD-L1 antibody was superior to each of the compounds individually. The efficacy models used by Gurney et al. were artificial models consisting of engineered cell lines overexpressing B7-H4. Activity in models with non-engineered B7-H4 expression levels that are more representative for B7-H4 levels in human tumors remains unclear. No observations on tolerability in mice were reported.

Michaelson et al. set out to construct a bispecific antibody targeting TRAIL-R2 and LTBR to explore the possibility that the bispecific antibody might trigger an enhanced, synergistic, or broader anti-tumor response than that achieved by treating with a mixture of the two antibodies (Michaelson et al., MAbs 1:128-41 (2009)). TRAIL-R2 is a TNF family receptor that is widely expressed in normal tissue including colon, lung, liver and brain (Spierings et al., J. Histochem. Cytochem. 52:821-31 (2004)) but also found co-expressed with LTBR on the surface of human epithelial cancer cell lines. The bispecific construct showed enhanced activity relative to the parental antibodies in vitro and in murine tumor xenograft models. No observations on tolerability in mice were reported.

These studies indicate a potential of targeting LTBR for tumor immunotherapy and suggest that activating LTBR signaling can enhance immune cell infiltration, induce TLS in the tumor environment, and potentially help to overcome resistance to checkpoint inhibition therapy.

The immune system is tightly regulated to ensure immune-mediated eradication of pathogens without causing tissue damage or autoimmunity. It is commonly observed that systemic immune modulating therapies tip this fine balance out of equilibrium and cause immune-related adverse events such as pneumonitis, colitis, hepatitis, thyroid disfunction, skin reactions, eye inflammation and others, representing a challenge for the development of novel immunotherapies, particularly in the context of combination therapies where toxicities may be additive or synergistic.

Due to the broad expression of LTBR in the organism, an agonistic LTBR-targeting drug capable of inducing TLS and creating an activating immune environment bears a substantial risk of causing systemic immune-related adverse events. Interestingly, Johansson-Percival et al. reported weight loss in mice after systemic administration of an LTBR activating compound, VTP-LIGHT (Johansson-Percival et al., Nat. Immunol. 18:1207-17 (2017)). Therefore, as postulated in the prior art, a therapeutic modality which activates LTBR specifically in the tumor but not in other tissues is needed to reduce the risk of toxicity and to generate a well tolerated drug that can be employed for combination therapies (Allen et al., Oncotarget 8:99207-8 (2017); Tang et al., Cell Mol. Immunol. 14:809-18 (2017))

While several groups have investigated LTBR as therapeutic target using various LTBR-targeting moieties, so far therapeutic modalities capable of LTBR activation specifically in the tumor have not been described.

### BRIEF SUMMARY OF THE INVENTION

Provided herein are multispecific binding molecules, such as bispecific antibodies, capable of activating lymphotoxin beta receptor (LTBR) specifically in a tumor. The multispecific binding molecules have a first specificity for LTBR and a second specificity for extra-domain B of fibronectin (EDB). EDB is a tumor-associated antigen (TAA) of the extracellular matrix. The multispecific binding molecules activate LTBR in tumors expressing EDB, but do not or only modestly activate LTBR in the absence of EDB, to an extent well below that of its ligands LIGHT and LTα1132, thereby reducing the risk of an immune related undesired event. Unlike LTBR activating molecules previously described, efficient LTBR activation in the presence of EDB is achieved upon EDB binding via the EDB specific part of the multispecific binding molecules of the invention and LTBR binding via the LTBR specific part of the multispecific binding molecules of the invention. If the EDB is not present, the multispecific binding molecule will not result in the activation of LTBR in normal tissue. This is a significant advantage over molecules described in the prior art that are based on natural LTBR ligands, e.g. LIGHT-antibody fusions, which can activate LTBR independent of a TAA, and thus are much less tumor specific for activation of LTBR as compared to the molecules of the invention, as shown in the examples herein.

Provided herein are multispecific binding molecules. The multispecific binding molecules can comprise (i) a first binding domain that specifically binds to a lymphotoxin beta receptor (LTBR), and (ii) a second binding domain that specifically binds to EDB, wherein the multispecific binding molecule activates LTBR upon binding of the EDB. More specifically, the multispecific binding molecule activates LTBR when the multispecific binding molecule simultaneously binds the LTBR and the EDB, via its respective specific binding domains for these targets. Preferably this happens in a tumor environment wherein cells expressing LTBR and the cells expressing the EDB are present, resulting in specific activation of LTBR in tumor tissue. In certain embodiments, the multispecific binding molecule activates LTBR in a tumor specific manner. The multispecific binding molecule can, for example, be a bispecific antibody. In certain embodiments, the multispecific binding molecule comprises two antigen binding domains. In certain embodiments, the multispecific binding molecule comprises three antigen binding domains. The three antigen binding domains can, for example, comprise one binding domain that specifically binds to LTBR. The three antigen binding domains can, for example, comprise two binding domains that specifically bind EDB.

In certain embodiments, the multispecific binding molecule comprises three antigen binding domains and is comprised of an antibody (e.g. in IgG format) to which an additional binding domain, e.g. in the form of a single chain variable domain, has been fused, e.g. to the N- or C-terminus of the heavy or of the light chain of the antibody.

For multispecific binding molecules of the invention that specifically bind LTBR and a TAA present in the extracellular matrix, the TAA present in the extracellular matrix is fibronectin. Preferably, a binding domain that binds to the TAA specifically binds to extra domain B of fibronectin (EDB).

In certain non-limiting embodiments, the binding domain that specifically binds to LTBR comprises a BHA10 antibody or of a CBE11 antibody or a fragment or derivative thereof, for instance a single chain antibody fragment (scFv), comprising a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said VH and VL comprise any of the following:

(i) HCDR1, HCDR2 and HCDR3 comprising the amino acid sequences of SEQ ID NO:60, SEQ ID NO:61, and SEQ ID NO:62, respectively, and LCDR1, LCDR2 and LCDR3 comprising the amino acid sequences of SEQ ID NO:63, SEQ ID NO:64, and SEQ ID NO:65, respectively; or
(ii) HCDR1, HCDR2 and HCDR3 comprising the amino acid sequences of SEQ ID NO:83, SEQ ID NO:61, and SEQ ID NO:62, respectively, and LCDR1, LCDR2 and LCDR3 comprising the amino acid sequences of SEQ ID NO:63, SEQ ID NO:64, and SEQ ID NO:65, respectively; or
(iii) HCDR1, HCDR2 and HCDR3 comprising the amino acid sequences of SEQ ID NO:66, SEQ ID NO:67, and SEQ ID NO:68, respectively, and LCDR1, LCDR2, and LCDR3 comprising the amino acid sequences of SEQ ID NO:69, SEQ ID NO:70, and SEQ ID NO:71, respectively; or
(iv) VH comprises an amino acid sequence having at least 95%, 96%, 97%, 98%, 99% identity or 100% identity to the amino acid sequence of SEQ ID NO:43, and VL comprises an amino acid sequence having at least 95%, 96%, 97%, 98%, 99% identity or 100% identity to the amino acid sequence of SEQ ID NO:44, such as wherein VH comprises an amino acid sequence having at least 95% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 96% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 97% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 98% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 99% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having 100% identity to the amino acid sequence of SEQ ID NO:43, and VL comprises an amino acid sequence having at least 95%, 96%, 97%, 98%, 99% identity or 100% identity to the amino acid sequence of SEQ ID NO:44; or
(v) VH comprises an amino acid sequence having at least 95%, 96%, 97%, 98%, 99% identity or 100% identity to the amino acid sequence of SEQ ID NO:47, and VL comprises an amino acid sequence having at least 95%, 96%, 97%, 98%, 99% identity or 100% identity to the amino acid sequence of SEQ ID NO:48, such as wherein VH comprises an amino acid sequence having at least 95% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 96% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 97% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 98% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 99% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having 100% identity to the amino acid sequence of SEQ ID NO:47, and VL comprises an amino acid sequence having at least 95%, 96%, 97%, 98%, 99% identity or 100% identity to the amino acid sequence of SEQ ID NO:48; or

### (vi) SEQ ID NO: 22; or

(vii) SEQ ID NO: 23; or
(viii) SEQ ID NO: 25.

In certain non-limiting embodiments, the second binding domain that specifically binds to EDB comprises an L19 antibody or a fragment or derivative thereof, for instance comprising a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said antibody or fragment thereof comprise any of the following:

(i) HCDR1, HCDR2 and HCDR3 comprising the amino acid sequences of SEQ ID NO:72, SEQ ID NO:73, and SEQ ID NO:74, respectively, and LCDR1, LCDR2 and LCDR3 comprising the amino acid sequences of SEQ ID NO:75, SEQ ID NO:76, and SEQ ID NO:77, respectively; or
(ii) VH comprises an amino acid sequence having at least 95%, 96%, 97%, 98%, 99% identity or 100% identity to the amino acid sequence of SEQ ID NO:45, and VL comprises an amino acid sequence having at least 95%, 96%, 97%, 98%, 99% identity or 100% identity to the amino acid sequence of SEQ ID NO:46, such as wherein VH comprises an amino acid sequence having at least 95% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 96% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 97% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 98% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 99% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having 100% identity to the amino acid sequence of SEQ ID NO:45, and VL comprises an amino acid sequence having at least 95%, 96%, 97%, 98%, 99% identity or 100% identity to the amino acid sequence of SEQ ID NO:46.

In certain non-limiting embodiments, the multispecific binding molecule comprises:

(1) a binding domain that specifically binds to LTBR comprises a BHA10 antibody or of a CBE11 antibody or a fragment or derivative thereof, for instance a single chain antibody fragment (scFv), comprising a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said VH and VL comprise any of the following:
(i) HCDR1, HCDR2 and HCDR3 comprising the amino acid sequences of SEQ ID NO:60, SEQ ID NO:61, and SEQ ID NO:62, respectively, and LCDR1, LCDR2 and LCDR3 comprising the amino acid sequences of SEQ ID NO:63, SEQ ID NO:64, and SEQ ID NO:65, respectively; or
(ii) HCDR1, HCDR2 and HCDR3 comprising the amino acid sequences of SEQ ID NO:83, SEQ ID NO:61, and SEQ ID NO:62, respectively, and LCDR1, LCDR2 and LCDR3 comprising the amino acid sequences of SEQ ID NO:63, SEQ ID NO:64, and SEQ ID NO:65, respectively; or
(iii) HCDR1, HCDR2 and HCDR3 comprising the amino acid sequences of SEQ ID NO:66, SEQ ID NO:67, and SEQ ID NO:68, respectively, and LCDR1, LCDR2, and LCDR3 comprising the amino acid sequences of SEQ ID NO:69, SEQ ID NO:70, and SEQ ID NO:71, respectively; or
(iv) VH comprises an amino acid sequence having at least 95%, 96%, 97%, 98%, 99% identity or 100% identity to the amino acid sequence of SEQ ID NO:43, and VL comprises an amino acid sequence having at least 95%, 96%, 97%, 98%, 99% identity or 100% identity to the amino acid sequence of SEQ ID NO:44, such as wherein VH comprises an amino acid sequence having at least 95% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 96% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 97% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 98% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 99% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having 100% identity to the amino acid sequence of SEQ ID NO:43, and VL comprises an amino acid sequence having at least 95%, 96%, 97%, 98%, 99% identity or 100% identity to the amino acid sequence of SEQ ID NO:44; or
(v) VH comprises an amino acid sequence having at least 95%, 96%, 97%, 98%, 99% identity or 100% identity to the amino acid sequence of SEQ ID NO:47, and VL comprises an amino acid sequence having at least 95%, 96%, 97%, 98%, 99% identity or 100% identity to the amino acid sequence of SEQ ID NO:48, such as wherein VH comprises an amino acid sequence having at least 95% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 96% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 97% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 98% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 99% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having 100% identity to the amino acid sequence of SEQ ID NO:47, and VL comprises an amino acid sequence having at least 95%, 96%, 97%, 98%, 99% identity or 100% identity to the amino acid sequence of SEQ ID NO:48; or

### (vi) SEQ ID NO: 22; or

(vii) SEQ ID NO: 23; or
(viii) SEQ ID NO: 25; and
(2) a second binding domain that specifically binds to EDB comprises an L19 antibody or a fragment or derivative thereof, for instance comprising a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said antibody or fragment thereof comprise HCDR1, HCDR2 and HCDR3 comprising the amino acid sequences of SEQ ID NO:72, SEQ ID NO:73, and SEQ ID NO:74, respectively, and LCDR1, LCDR2 and LCDR3 comprising the amino acid sequences of SEQ ID NO:75, SEQ ID NO:76, and SEQ ID NO:77, respectively.

In certain non-limiting embodiments, the multispecific binding molecule comprises:

(1) a binding domain that specifically binds to LTBR comprises a BHA10 antibody or of a CBE11 antibody or a fragment or derivative thereof, for instance a single chain antibody fragment (scFv), comprising a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said VH and VL comprise any of the following:
(i) HCDR1, HCDR2 and HCDR3 comprising the amino acid sequences of SEQ ID NO:60, SEQ ID NO:61, and SEQ ID NO:62, respectively, and LCDR1, LCDR2 and LCDR3 comprising the amino acid sequences of SEQ ID NO:63, SEQ ID NO:64, and SEQ ID NO:65, respectively; or
(ii) HCDR1, HCDR2 and HCDR3 comprising the amino acid sequences of SEQ ID NO:83, SEQ ID NO:61, and SEQ ID NO:62, respectively, and LCDR1, LCDR2 and LCDR3 comprising the amino acid sequences of SEQ ID NO:63, SEQ ID NO:64, and SEQ ID NO:65, respectively; or
(iii) HCDR1, HCDR2 and HCDR3 comprising the amino acid sequences of SEQ ID NO:66, SEQ ID NO:67, and SEQ ID NO:68, respectively, and LCDR1, LCDR2, and LCDR3 comprising the amino acid sequences of SEQ ID NO:69, SEQ ID NO:70, and SEQ ID NO:71, respectively; or
(iv) VH comprises an amino acid sequence having at least 95%, 96%, 97%, 98%, 99% identity or 100% identity to the amino acid sequence of SEQ ID NO:43, and VL comprises an amino acid sequence having at least 95%, 96%, 97%, 98%, 99% identity or 100% identity to the amino acid sequence of SEQ ID NO:44, such as wherein VH comprises an amino acid sequence having at least 95% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 96% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 97% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 98% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 99% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having 100% identity to the amino acid sequence of SEQ ID NO:43, and VL comprises an amino acid sequence having at least 95%, 96%, 97%, 98%, 99% identity or 100% identity to the amino acid sequence of SEQ ID NO:44; or
(v) VH comprises an amino acid sequence having at least 95%, 96%, 97%, 98%, 99% identity or 100% identity to the amino acid sequence of SEQ ID NO:47, and VL comprises an amino acid sequence having at least 95%, 96%, 97%, 98%, 99% identity or 100% identity to the amino acid sequence of SEQ ID NO:48, such as where VH comprises an amino acid sequence having at least 95% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 96% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 97% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 98% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 99% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having 100% identity to the amino acid sequence of SEQ ID NO:47, and VL comprises an amino acid sequence having at least 95%, 96%, 97%, 98%, 99% identity or 100% identity to the amino acid sequence of SEQ ID NO:48; or

### (vi) SEQ ID NO: 22; or

(vii) SEQ ID NO: 23; or
(viii) SEQ ID NO: 25; and
(2) a second binding domain that specifically binds to EDB comprises an L19 antibody or a fragment or derivative thereof, for instance comprising a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said antibody or fragment thereof comprises a VH that comprises an amino acid sequence having at least 95%, 96%, 97%, 98%, 99% identity or 100% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 95% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 96% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 97% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 98% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 99% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having 100% identity to the amino acid sequence of SEQ ID NO:45, and VL comprises an amino acid sequence having at least 95%, 96%, 97%, 98%, 99% identity or 100% identity to the amino acid sequence of SEQ ID NO:46.

In certain non-limiting embodiments, the multispecific molecule comprises:

(1) a binding domain that specifically binds to LTBR comprising SEQ ID NO: 22; and
(2) a second binding domain that specifically binds to EDB comprises an L19 antibody or a fragment or derivative thereof, for instance comprising a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said antibody or fragment thereof comprise any of the following:
(i) HCDR1, HCDR2 and HCDR3 comprising the amino acid sequences of SEQ ID NO:72, SEQ ID NO:73, and SEQ ID NO:74, respectively, and LCDR1, LCDR2 and LCDR3 comprising the amino acid sequences of SEQ ID NO:75, SEQ ID NO:76, and SEQ ID NO:77, respectively; or
(ii) VH comprises an amino acid sequence having at least 95%, 96%, 97%, 98%, 99% identity or 100% identity to the amino acid sequence of SEQ ID NO:45, and VL comprises an amino acid sequence having at least 95%, 96%, 97%, 98%, 99% identity or 100% identity to the amino acid sequence of SEQ ID NO:46.
In certain non-limiting embodiments, the multispecific molecule comprises:
(1) a binding domain that specifically binds to LTBR comprising SEQ ID NO: 23; and
(2) a second binding domain that specifically binds to EDB comprises an L19 antibody or a fragment or derivative thereof, for instance comprising a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said antibody or fragment thereof comprise any of the following:
(i) HCDR1, HCDR2 and HCDR3 comprising the amino acid sequences of SEQ ID NO:72, SEQ ID NO:73, and SEQ ID NO:74, respectively, and LCDR1, LCDR2 and LCDR3 comprising the amino acid sequences of SEQ ID NO:75, SEQ ID NO:76, and SEQ ID NO:77, respectively; or
(ii) VH comprises an amino acid sequence having at least 95%, 96%, 97%, 98%, 99% identity or 100% identity to the amino acid sequence of SEQ ID NO:45, and VL comprises an amino acid sequence having at least 95%, 96%, 97%, 98%, 99% identity or 100% identity to the amino acid sequence of SEQ ID NO:46.
In certain non-limiting embodiments, the multispecific molecule comprises: and
(1) a binding domain that specifically binds to LTBR comprising SEQ ID NO: 25;
(2) a second binding domain that specifically binds to EDB comprises an L19 antibody or a fragment or derivative thereof, for instance comprising a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said antibody or fragment thereof comprise any of the following:
(i) HCDR1, HCDR2 and HCDR3 comprising the amino acid sequences of SEQ ID NO:72, SEQ ID NO:73, and SEQ ID NO:74, respectively, and LCDR1, LCDR2 and LCDR3 comprising the amino acid sequences of SEQ ID NO:75, SEQ ID NO:76, and SEQ ID NO:77, respectively; or
(ii) VH comprises an amino acid sequence having at least 95%, 96%, 97%, 98%, 99% identity or 100% identity to the amino acid sequence of SEQ ID NO:45, and VL comprises an amino acid sequence having at least 95%, 96%, 97%, 98%, 99% identity or 100% identity to the amino acid sequence of SEQ ID NO:46.

In certain non-limiting embodiments, the multispecific binding molecule comprises any of the following:

(a) (i) a first heavy chain comprising the amino acid sequence of SEQ ID NO: 1 forming a binding domain with a first light chain comprising the amino acid sequence of SEQ ID NO: 2, and (ii) a second heavy chain comprising the amino acid sequence of SEQ ID NO: 4 forming a binding domain with a second light chain comprising the amino acid sequence of SEQ ID NO: 5 [multispecific binding molecule referred to as COVA14121]; or
(b) (i) a first heavy chain comprising the amino acid sequence of SEQ ID NO: 9 forming a binding domain with a first light chain comprising the amino acid sequence of SEQ ID NO: 10, and (ii) a second heavy chain comprising the amino acid sequence of SEQ ID NO: 4 forming a binding domain with a second light chain comprising the amino acid sequence of SEQ ID NO: 5 [multi specific binding molecule referred to as COVA14122].

In certain further non-limiting embodiments, the multispecific binding molecule comprises any of the following:

(c) (i) an scFv-heavy chain fusion comprising the amino acid sequence of SEQ ID NO: 30, the heavy chain part thereof (comprising SEQ ID NO: 84) forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 [multispecific binding molecule referred to as COVA1480]; or
(d) (i) an scFv-heavy chain fusion comprising the amino acid sequence of SEQ ID NO: 31, the heavy chain part thereof (comprising SEQ ID NO: 84) forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 [multispecific binding molecule referred to as COVA1481]; or
(e) (i) an scFv-heavy chain fusion comprising the amino acid sequence of SEQ ID NO: 32, the heavy chain part thereof (comprising SEQ ID NO: 84) forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 [multispecific binding molecule referred to as COVA1482]; or
(f) (i) an scFv-heavy chain fusion comprising the amino acid sequence of SEQ ID NO: 33, the heavy chain part thereof (comprising SEQ ID NO: 84) forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 [multispecific binding molecule referred to as COVA1483]; or
(g) (i) an scFv-heavy chain fusion comprising the amino acid sequence of SEQ ID NO: 34, the heavy chain part thereof (comprising SEQ ID NO: 84) forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 [multispecific binding molecule referred to as COVA14107]; or
(h) (i) an scFv-heavy chain fusion comprising the amino acid sequence of SEQ ID NO: 35, the heavy chain part thereof (comprising SEQ ID NO: 84) forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 [multispecific binding molecule referred to as COVA14108]; or
(j) (i) an scFv-heavy chain fusion comprising the amino acid sequence of SEQ ID NO: 38, the heavy chain part thereof (comprising SEQ ID NO: 3) forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 [multispecific binding molecule referred to as COVA14133]; or
(k) (i) an scFv-heavy chain fusion comprising the amino acid sequence of SEQ ID NO: 39, the heavy chain part thereof (comprising SEQ ID NO: 3) forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 [multispecific binding molecule referred to as COVA14174]; or
(l) (i) an scFv-heavy chain fusion comprising the amino acid sequence of SEQ ID NO: 56, the heavy chain part thereof (comprising SEQ ID NO: 84) forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 [multispecific binding molecule referred to as COVA1456].

In some embodiments the multispecific molecule comprises (i) an scFv-heavy chain fusion comprising the amino acid sequence of SEQ ID NO: 38, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5.

Also provided are one or more nucleic acid molecules encoding the multispecific binding molecules as disclosed herein. Also provided are one or more vectors comprising the one or more nucleic acid molecules as disclosed herein. Also provided is an isolated host cell comprising the one or more vectors as disclosed herein.

Also provided are pharmaceutical compositions comprising the multispecific binding molecule as disclosed herein and a pharmaceutically acceptable carrier.

Also provided are methods of treating cancer in a subject in need thereof. The methods comprise administering to the subject the multispecific binding molecule as disclosed herein, the one or more nucleic acid molecules as disclosed herein, the one or more vectors as disclosed herein, or the pharmaceutical composition as disclosed herein.

Also provided are uses of the multispecific binding molecule as disclosed herein, the one or more nucleic acid molecules as disclosed herein, the one or more vectors as disclosed herein, or the pharmaceutical composition as disclosed herein, for activating LTBR in tumor tissue.

Also provided are methods of producing a multispecific binding molecule as disclosed herein, the method comprising expressing the one or more nucleic acid molecules as disclosed herein or the one or more vectors of as disclosed herein in a host cell and harvesting the multispecific binding molecule.

For the multispecific binding molecules of the invention, the binding domain for the first antigen binds to LTBR on cells present in tumors (e.g., tumor cells, fibroblasts, monocytes, etc.). The binding domain for the second antigen binds to EDB, which is a tumor associated antigen (TAA) of an extracellular matrix present in tumors.

In certain embodiments, the multispecific binding molecule, such as bispecific antibody, or antigen binding fragment thereof comprises two heavy chains (HCs) and two light chains (LCs) to form two binding domains for EDB.

In certain embodiments, an scFv is fused to a carboxy (C)-terminal or an amino (N)-terminal end of one HC. In certain embodiments, the scFv fused to the HC comprises an amino acid sequence selected from SEQ ID NO:30, SEQ ID NO:31, SEQ ID NO:32, SEQ ID NO:33, SEQ ID NO:34, SEQ ID NO:35, SEQ ID NO:38, SEQ ID NO:39, or SEQ ID NO:56.

Also provided are isolated nucleic acids encoding the scFv fused to the HC of the isolated anti-LTBR bispecific antibody or antigen binding fragment thereof as disclosed herein. Also provided are isolated nucleic acids encoding the HC and LC of the anti-LTBR bispecific antibody or antigen binding fragment thereof as disclosed herein.

In certain embodiments, the heavy chains, light chains, and/or functional fragments thereof such as the antigen-specific binding domains are human, or humanized.

Also provided are nucleic acids encoding the heavy chains, light chains, and/or functional fragments thereof of the multispecific binding molecules as disclosed herein.

Also provided are vectors comprising the nucleic acids as disclosed herein.

Also provided are host cells comprising the nucleic acids or vectors as disclosed herein.

In preferred embodiments, the multispecific binding molecules, bispecific antibodies, nucleic acids, vectors, or host cells, according to the invention are isolated multispecific binding molecules, isolated bispecific antibodies, isolated nucleic acids, isolated vectors, or isolated host cells, respectively.

Also provided are pharmaceutical compositions comprising a multispecific binding molecule, such as a bispecific antibody, or antigen binding fragment thereof as disclosed herein and a pharmaceutically acceptable carrier.

Also provided are methods of treating a cancer in a subject in need thereof. The methods comprise (a) identifying a subject in need of cancer treatment; and (b) administering to the subject in need thereof the multispecific binding molecule, e.g. in the form of a pharmaceutical composition, of the invention, wherein administering the pharmaceutical composition to the subject in need thereof treats the cancer in the subject.

Also provided are methods of activating a LTBR-expressing cell. The methods comprise contacting the LTBR-expressing cell with the multispecific binding molecule, e.g. in the form of a pharmaceutical composition, of the invention, wherein contacting the LTBR-expressing cell with the multispecific binding molecule or pharmaceutical composition results in an increase in RANTES, IL-6, IL-8, MIP-3b, ICAM-1, I-TAC, IP-10, IL-12p70, TNF-α, MIP-3a, and/or SDF-1a expression as compared to a cell expressing LTBR in an environment where EDB is absent.

Also provided are methods of inhibiting growth or proliferation of cancer cells in tumors that express EDB. The methods comprise contacting the cancer cells and/or cells in the tumor microenvironment with the multispecific binding molecule, e.g. in the form of a pharmaceutical composition, of the invention, wherein contacting the cancer cells and/or the cells in the tumor microenvironment with the pharmaceutical composition inhibits growth or proliferation of the cancer cells.

Also provided are methods of producing a pharmaceutical composition as disclosed herein. The methods comprise combining the isolated multispecific binding molecule, e.g. bispecific antibody, or antigen binding fragment thereof, of the invention with a pharmaceutically acceptable carrier to obtain the pharmaceutical composition.

Also provided are methods of making a multispecific binding molecule such as bispecific antibody or antigen binding fragment thereof. The methods comprise culturing a host cell comprising the nucleic acids as disclosed herein under conditions to produce the multispecific binding molecule such as bispecific antibody or antigen binding fragment thereof and recovering the multispecific binding molecule such as bispecific antibody or antigen binding fragment thereof.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-1A6 show schematic representations of anti-LTBR bispecific antibodies and of control molecules. FIGS. 1A-1D show control monoclonal antibodies with silencing Fc mutations IgG1σ. FIGS. 1E-1F show 1:1 Knob-into-Hole (KiH) heterodimers comprising targeting arm (B21M or EDBmAb1) and human LIGHT fused to Fc. A set of mutations were introduced in the Fc fused to human LIGHT to abrogate binding to protein A and favor purification of heterodimer. FIGS. 1G-1J show human LTα1β2 antibody fusions. FIGS. 1K-10 show 1:1 KiH heterodimers. FIGS. 1P-1S show 2:1 heterodimers, isotype control antibody fused to stapled scFv derived from LTBRmAb1. FIGS. 1T-1W show 2:1 heterodimers, EDBmAb1 fused to stapled scFv derived from LTBRmAb1. FIGS. 1X-1Y show 2:1 heterodimers, EDBmAb1 fused to stapled scFv derived from lower affinity variants of LTBRmAb1. FIGS. 1Z-1A1 show 2:1 heterodimers, EDBmAb1 or B21M fused to stapled scFv derived from LTBRmAb1, without protein A mutations in the Fc region. FIGS. 1A2-1A5 show 2:1 heterodimers, EDBmAb1 or B21M fused to disulfide-stabilized scFv derived from LTBRmAb1. FIG. 1A6 shows a 2:1 heterodimer, MSLNmAb1 fused to stapled scFv derived from LTBRmAb1.

FIGS. 2A-2G show size exclusion chromatograms (SECs) of: FIG. 2A: COVA1418 consisting of 3×hmLIGHT-Fc with the heavy chain and light chain of an anti-RSV antibody B21M; FIG. 2B: COVA1454 consisting of 3×hmLIGHT-Fc with the heavy chain and light chain of an anti-EDB antibody EDBmAb1; FIG. 2C: COVA14133 consisting of an anti-EDB antibody EDBmAb1 heavy chain carrying a C-terminal stapled scFv BHA10 (VH-VL orientation) fusion with the heavy chain and light chain of an anti-EDB antibody EDBmAb1; FIG. 2D: COVA14113 consisting of the EDBmAb1 heavy chain carrying a C-terminal LTα1β2 fusion with the light chain of an anti-EDB antibody EDBmAb1; FIG. 2E: COVA14114 consisting of the anti-RSV B21M antibody heavy chain carrying a C-terminal LTα1β2 fusion with the light chain of an anti-RSV B21M antibody; FIG. 2F: COVA14116 consisting of the EDBmAb1 heavy chain carrying a C-terminal LTα1β2 fusion with the heavy chain and light chain of an anti-EDB antibody EDBmAb1; FIG. 2G: COVA14117 consisting of the anti-RSV B21M antibody heavy chain carrying a C-terminal LTα1β2 fusion with the heavy chain and light chain of the anti-RSV B21M antibody.

FIGS. 3A-3D show graphs demonstrating the results of A549 NF-κB reporter assays. FIG. 3A: Tumor associated antigen (TAA)—dependent activation of LTBR by COVA1454 compared to COVA1418 and recombinant human LIGHT; FIG. 3B: TAA—independent activation of LTBR by COVA1454 compared to COVA1418 and recombinant human LIGHT; FIG. 3C: TAA—dependent activation of LTBR by COVA14113 and COVA14116 compared to recombinant human LIGHT and recombinant human LTα1β2; FIG. 3D: TAA—independent activation of LTBR by COVA14113 and COVA14116 compared to recombinant human LIGHT and recombinant human LTα1β2.

FIGS. 4A-4D show graphs demonstrating the results of A549 NF-κB reporter assays using 1:1 heterodimers consisting of EDBmAb1 and LTBRmAb1 or LTBRmAb2. FIG. 4A: Tumor associated antigen (TAA)—dependent activation of LTBR by COVA14121 compared to COVA14120, COVA14124, COVA1413, COVA1440, and recombinant human LIGHT; FIG. 4B: TAA—independent activation of LTBR by COVA14121 compared to COVA14120, COVA14124, COVA1413, COVA1440, and recombinant human LIGHT; FIG. 4C: TAA—dependent activation of LTBR by COVA14122 compared to COVA14123, COVA14124, COVA1402, COVA1440, and recombinant human LIGHT; FIG. 4D: TAA—independent activation of LTBR by COVA14122 compared to COVA14123, COVA14124, COVA1402, COVA1440, and recombinant human LIGHT.

FIGS. 5A-5E show graphs demonstrating the results of A549 NF-κB reporter assays using 2:1 bispecific antibodies. FIG. 5A: Efficient activation of LTBR by COVA1456 (2:1 EDBmAb1×LTBR mAB1) in the presence of EDB-containing fibronectin. No LTBR activation observed with isotype control molecule COVA1462 (2:1 B21M×LTBR mAb1); FIG. 5B: No LTBR activation measured in absence of EDB-containing fibronectin by COVA1456 or its isotype control molecule COVA1462; FIG. 5C: Comparison of TAA-dependent LTBR activation by COVA1456 with COVA1482, their respective control molecules COVA1462 and COVA1486, and recombinant human LIGHT; FIG. 5D: Comparison of TAA-dependent LTBR activation by COVA1482 and bispecifics containing lower affinity variants of LTBRmAb1 COVA14107 and COVA14108, and COVA1486; FIG. 5E: Comparison of TAA-dependent LTBR activation by COVA1482 and COVA14133 (construct without protein A mutations), and their respective control molecules COVA1486 and COVA14136; FIG. 5F: Efficient activation of LTBR by COVA14133 (2:1 EDBmAb1×LTBR mAB1) and COVA14116 (2:1 EDBmAb1 x LTα1β2) in the presence of EDB-containing fibronectin. No LTBR activation observed with isotype control molecule COVA14136 (2:1 B21M×LTBR mAb1). TAA-independent activation of LTBR by COVA14117 (2:1 B21M×LTα1β2); FIG. 5G: No LTBR activation measured in absence of EDB-containing fibronectin by COVA14133 or its isotype control molecule COVA14136. TAA-independent activation of LTBR by COVA14116 and COVA14117.

FIG. 6 shows the results of flow cytometry staining of ICAM-1 on A375 cells after co-culture experiment. COVA1482 and its control molecule COVA1486 are compared to recombinant human LIGHT.

FIGS. 7A-7J show graphs demonstrating measurements of cytokines in supernatants of co-cultures treated with anti-EDB/anti-LTBR bispecific antibodies COVA14133 compared to COVA14136 and COVA1440. Assays are performed using the MSD platform. FIG. 7A: Concentrations of human RANTES; FIG. 7B: Concentrations of human IL-6; FIG. 7C: Concentrations of human IL-8; FIG. 7D: Concentrations of human MIP-3b. Graphs shown in FIGS. 7E-J include in addition the 2:1 antibody×LTα1β2 fusions COVA14116 and COVA14117 as well as EDBmAb1 COVA1452. FIG. 7E: Concentrations of human IP-10; FIG. 7F: Concentrations of human SDF-1a; FIG. 7G: Concentrations of human IL-12p70; FIG. 7H: Concentrations of human I-TAC; FIG. 7I: Concentrations of human MIP-3a; FIG. 7J: Concentrations of human TNFα.

FIGS. 8A-8B show LTBR activation by a MSLN/LTBR bispecific in A549 NF-κB reporter/CHOK1MSLN or A549 NF-κB reporter/H226 co-culture cell assays. FIG. 8A: Activation of LTBR in A549 NF-κB reporter/H226 co-culture assay. COVA14146 (2:1 MSLNmAb1×LTBRmAb1) compared to LIGHT and to the isotype control 2:1 constructs COVA1486; FIG. 8B: Concentration of secreted RANTES upon activation of LTBR in A549 NF-κB reporter/H226 co-culture assay. COVA14146 (2:1 MSLNmAb1×LTBRmAb1) compared to LIGHT and to the isotype control 2:1 constructs COVA1486.

FIGS. 9A-9B show the schematic representations of possible LTBR activation mechanisms. FIG. 9A: In presence of EDB (a tumor associated antigen (TAA)) in the extracellular matrix, the bispecific antibody can cluster LTBR on the cell surface via binding to EDB. Activation of LTBR leads to secretion of chemoattractant cytokines and chemokines. FIG. 9B: In absence of EDB in the extracellular matrix, clustering of LTBR does not take place. As a consequence, no activation of LTBR can take place.

FIG. 10 shows the migration of PBMCs towards cytokines induced by LTBR activation. Supernatants of co-cultures (as shown in FIG. 7) treated with anti-EDB/anti-LTBR bispecific antibodies COVA14133 compared to COVA14136 and COVA1440, and anti-EDB/LTα1β2 fusion COVA14116 compared to COVA14117, acted as attractant for PBMCs in a transwell migration assay. The number of PBMCs that migrated towards the co-culture supernatants were counted and are shown in the graph. Migration of PBMCs was induced in a dose-dependent manner towards supernatants from co-cultures stimulated with COVA14133, COVA14116 and to a bit lesser extend COVA14117. Supernatants from co-cultures incubated with non-targeted control molecule COVA14136 did not induce migration of PBMCs.

FIGS. 11A-11B shows the adhesion and transmigration of monocytes to HUVECs monolayers stimulated with anti-EDB/anti-LTBR bispecific antibody COVA14133 compared to its control COVA14136. In an imaging-based assay consisting of a continuous flow of monocytes across HUVEC monolayers grown in the presence of EDB and stimulated with 50 nM COVA14133 or COVA14136, the number of adherent (A) and transmigrated (B) monocytes was counted over time. Student T-test analyses of COVA14133 against COVA14136 was conducted and marked as follows: *P<0.05, ** P<0.01, *** P<0.005.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., preferably a human.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., anti-LTBR bispecific antibodies and polynucleotides that encode them, anti-LTBR/anti-EDB bispecific antibodies and polynucleotides that encode them, LTBR polypeptides and LTBR polynucleotides that encode them, EDB polypeptides and EDB polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected with a nucleic acid molecule of the invention. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed multispecific binding molecule, e.g. bispecific antibody, can be within the cytoplasm of a host cell, into the extracellular milieu such as the growth medium of a cell culture or anchored to the cell membrane. Preferably the multispecific binding molecule is secreted from production host cells into the culture medium.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

A “multispecific binding molecule” as used herein means a molecule that specifically binds to at least two different molecules. Preferably, the molecule is a protein, for instance comprising an antibody or fragment or derivative thereof. A multispecific binding molecule or antibody of the invention has at least one binding domain specifically binding to LTBR, and at least one binding domain specifically binding to EDB of fibronectin, and in view of the presence of a binding specificity towards LTBR is sometimes referred to as “anti-LTBR” binding molecule or antibody herein.

A “binding domain” as used herein means a functional part of a binding molecule, e.g. from an antibody, that confers specific binding of the binding molecule to a target molecule. Examples of binding domains are variable regions of antibodies that confer specific binding to a target molecule, and may be formed by more than one chain of an antibody, e.g. the variable domain of a heavy chain paired to the variable domain of a light chain, or by a single chain such as in scFv molecules, or e.g. a single domain such as VHH from llamas, e.g. nanobodies, etc. The target molecule of the present invention is LTBR or fibronectin, in particular EDB of fibronectin.

The term “specific binding” as used herein refers to antibody binding to a predetermined antigen with greater affinity than for other antigens. Typically, the antibody binds to a predetermined antigen with a dissociation constant (K_{D}) of about 1×10⁻⁷ M or less, for example about 1×10⁻⁸M or less, about 1×10⁻⁹M or less, about 1×10⁻¹⁰ M or less, about 1×10⁻¹¹ M or less, about 1×10¹² M or less, about 1×10¹³ M or less or about 1×10¹⁴ M or less, typically with a K_{D} that is at least ten fold less than its K_{D} for binding to a non-specific antigen or epitope (e.g., BSA, casein). The dissociation constant can be measured using standard procedures. Antibodies that specifically bind to a predetermined antigen may, however, have cross-reactivity to other related antigens, for example to the same predetermined antigen from other species (homologs), such as human or monkey, for example Macaca fascicularis (cynomolgus, cyno) or Pan troglodytes (chimpanzee, chimp).

The term “tumor associated antigen” or “TAA” as used herein means antigens present on tumor cells or present in the extracellular matrix of tumors, which antigens are not qualitatively different form antigens found on normal cells or in extracellular matrix of normal tissues, but which are quantitatively different in some respect, e.g. they are present on tumor cells or in the extracellular matrix of tumors in significantly greater amounts, in higher density, at a different site of expression, and/or are differentially accessible to the immune system, etc. In certain embodiments, the tumor associated antigen is present on tumor cells or in the tumor extracellular matrix in an at least two times higher amount as on non-tumor cells or extracellular matrix, more preferably an at least five times higher amount, such as e.g. an at least 10-times higher amount, even more preferably an at least 100-times higher amount, such as e.g. an at least 1000-times higher amount and most preferably an at least 10′000-times higher amount. EDB is present in fibronectin in the extracellular matrix of tumor tissue, whereas it is typically not detectable in fibronectin forms that are present in normal tissue (i.e. the same tissue under normal conditions and not being in a tumor environment).

The term “extracellular matrix” as used herein means a non-cellular component present within all tissues and organs in the form of a three-dimensional network of extracellular macromolecules, such as collagen, enzymes, and glycoproteins, that provide structural and biochemical support of surrounding cells. Its exact composition varies for different tissues, but it is generally made up of proteoglycans, water, minerals, and fibrous proteins. A proteoglycan is composed of a protein core surrounded by long chains of starch-like molecules called glycosaminoglycans. Two main classes of extracellular matrix molecules make up the matrix: proteoglycans, and fibrous proteins, including for example collagen, elastin, fibronectin, and laminin.

### Antibodies

The invention generally relates to anti-LTBR multispecific binding molecules, nucleic acids and expression vectors encoding the multispecific binding molecules, recombinant cells containing the vectors, and compositions comprising the multispecific binding molecules. In preferred embodiments, the anti-LTBR multispecific binding molecules are anti-LTBR multispecific antibodies, such as anti-LTBR bispecific antibodies or antigen binding fragments thereof. In certain embodiments, the anti-LTBR multispecific binding molecules can comprise binding domains specifically binding to LTBR which binding domains are in a different format than antibodies or functional fragments thereof, e.g. they may comprise anti-LTBR Fynomers, anti-LTBR affimers, anti-LTBR darpins, and/or other protein scaffolds screened for candidates that specifically bind to LTBR. In multispecific binding molecules of the invention, the binding domain with specificity towards LTBR is not provided by LIGHT or LTα1β2 (natural ligands of LTBR), nor functional fragments or derivatives thereof such as 3×hmLIGHT. In preferred embodiments a binding domain with specificity towards LTBR in the multispecific binding molecules of the invention comprise an antibody against LTBR, preferably an agonistic antibody against LTBR, or a functional fragment or derivative thereof, such as an scFv. Agonistic antibodies against LTBR as such have been described, and non-limiting examples are BHA10 (e.g. WO2004002431), and CBE11 (e.g. WO0230986), or can alternatively be generated according to known methods for antibody generation, such as immunization of mice, phage display, etc.

Fyn SH3-derived polypeptides or Tynomers' are well known in the art and have been described e.g. in Grabulovski et al. (2007) JBC, 282, p. 3196-3204; WO 2008/022759; Bertschinger et al (2007) Protein Eng Des Sel 20(2):57-68; and Gebauer and Skerra (2009) Curr Opinion in Chemical Biology 13:245-255. The term “Fyn SH3-derived polypeptide”, used interchangeably herein with the term “Fynomer”, refers to a non-immunoglobulin-derived binding polypeptide (e.g. a so-called scaffold as described in Gebauer and Skerra (2009) Curr Opinion in Chemical Biology 13:245-255) derived from the human Fyn SH3 domain. Fynomers are small about 7-kDa globular polypeptides. The SH3 domain of the human Fyn kinase was successfully used as a scaffold to engineer proteins (Fyn SH3-derived binding proteins termed Fynomers) that bind with high affinity and specificity to different target proteins (WO 2008/022759, WO 2011/023685, WO 2013/135588, WO 2014/170063, Grabulovski D. et al., (2007) J Biol Chem 282, p. 3196-3204, Bertschinger J. et al. (2007) Protein Eng Des Sel, 20, p. 57-68, and Schlatter et al. (2012) mAbs, 4(4) p. 497-50).

Affimer molecules are small proteins (12-14 kDa) that bind to target molecules with similar specificity and affinity to that of antibodies. These engineered non-antibody binding proteins are designed to mimic the molecular recognition characteristics of monoclonal antibodies in different applications (see e.g. Tiede et al, eLife 2017, DOI: 10.7554/eLife.24903).

DARPins (for designed ankyrin repeat proteins) are genetically engineered antibody mimetic proteins, typically exhibiting highly specific protein binding, and are derived from natural ankyrin proteins. They consist of at least three repeat motifs, and typically their molecular mass is about 14 or 18 kDa for four- or five-repeat DARPins, respectively. DARPin designs were for instance described in Binz et al, 2003, J. Mol. Biol. 332: 489-503.

Other binding protein formats, such as protein scaffolds, are known in the art and can also be used to provide one or more binding domains of certain embodiments of multispecific binding molecules of the invention.

In preferred embodiments of the invention, the binding domain that binds to LTBR activates LTBR upon binding and is derived from an antibody, preferably an agonistic antibody, that specifically binds to LTBR. In specific embodiments, the binding domain that binds to LTBR is a single chain variable domain (scFv) of an antibody, which scFv can be in any available format, e.g. stabilized by methods described previously and/or herein.

The invention in certain embodiments relates to anti-LTBR/anti-EDB bispecific antibodies or antigen-binding fragments thereof, nucleic acids and expression vectors encoding the antibodies, recombinant cells containing the vectors, and compositions comprising the bispecific antibodies. Methods of making the multispecific binding molecules and/or antibodies, and methods of using the multispecific binding molecules and/or antibodies to treat diseases, including cancer, are also provided. The multispecific binding molecules and/or antibodies disclosed herein possess one or more desirable functional properties, including but not limited to one or more of specific binding to LTBR and EDB, high specificity to LTBR and EDB, and/or the ability to treat or prevent cancer when administered alone or in combination with other anti-cancer therapies.

As used herein, the term “antibody” is used in a broad sense and includes immunoglobulin or antibody molecules including human, humanized, composite and chimeric antibodies and antigen binding domains that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies of the invention include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated bispecific antibody that specifically binds to LTBR is substantially free of bispecific antibodies that do not bind to LTBR; an isolated bispecific antibody that specifically binds to LTBR and/or EDB is substantially free of bispecific antibodies that do not bind to LTBR and/or EDB). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. The monoclonal antibodies of the invention can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene. In certain embodiments, monoclonal antibodies are produced by a recombinant host cell that expresses nucleic acid sequences encoding the antibody. Such a recombinant host cell can for instance be obtained by transfection of the nucleic acid sequences into a parent cell, e.g. a CHO cell. The recombinant host cell can be cultured under conditions conducive to expression of the antibody in the host cell, and the antibody can be isolated from the host cell, the culture medium, or both.

In certain embodiments, a multispecific binding molecule of the invention comprises an antibody or one or more antigen-binding fragments thereof. As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdab) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment of the heavy chain. According to other particular embodiments, the antigen-binding fragment comprises Fab and F(ab′). In some embodiments, the antigen binding fragments include IgG-like molecules with complementary CH3 domains to force heterodimerisation; recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule. In some embodiments, IgG-like molecules with complementary CH3 domains molecules include the Triomab/Quadroma (Trion Pharma/Fresenius Biotech), the Knobs-in-Holes (Genentech), CrossMAbs (Roche) and the electrostatically-matched (Amgen), the LUZ-Y (Genentech), the Strand Exchange Engineered Domain body (SEEDbody)(EMD Serono), the Biclonic (Merus) or the DuoBody (Genmab A/S, see e.g. Labrijn et al, 2013, PNAS 110: 5145-5150). In some embodiments, the antigen binding fragments include “Stapled single chain Fv” or “spFv” refers to a scFv that comprises one or more disulfide bonds between the VH and the linker or the VL and the linker. Typically the spFv may comprise one disulfide bond between the VH and the linker, one disulfide bond between the VL and the linker, or two disulfide bonds between the VH and the linker and the VL and the linker. scFv molecules which comprise disulfide bonds between the VH and the VL are excluded from the term “spFv”.

As used herein, the term “single-chain antibody” refers to a conventional single-chain antibody in the field, which comprises a heavy chain variable region and a light chain variable region connected by a short peptide, e.g. of about 15 to about 20 amino acids. As used herein, the term “single domain antibody” refers to a conventional single domain antibody in the field, which comprises a heavy chain variable region and a heavy chain constant region or which comprises only a heavy chain variable region.

In certain embodiments, multispecific binding molecules of the invention comprise an antibody with one or more mutations in the Fc that abrogate binding to protein A. Such mutations facilitate purification of heterodimer, and have for instance been described in WO2010151792.

As used herein, the term “human antibody” refers to an antibody produced by a human or an antibody having an amino acid sequence corresponding to an antibody produced by a human made using any technique known in the art. This definition of a human antibody includes intact or full-length antibodies, antigen-binding fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes do not overlap or do not substantially overlap. In an embodiment, the first and second epitopes are on different antigens, e.g., different proteins (or different subunits of a multimeric protein). In certain embodiments, a multispecific antibody comprises a third, fourth, or fifth immunoglobulin variable domain, or even more immunoglobulin variable domains. In an embodiment, a multispecific antibody is a bispecific antibody molecule, a trispecific antibody molecule, or a tetraspecific antibody molecule.

As used herein, the term “bispecifc antibody” refers to a multispecific antibody that binds no more than two epitopes, preferably no more than two antigens. A bispecific antibody is characterized by a first immunoglobulin variable domain which has binding specificity for a first epitope (e.g., an epitope on a LTBR antigen) and a second immunoglobulin variable domain that has binding specificity for a second epitope (e.g., an epitope on EDB). In an embodiment, a bispecific antibody comprises a first heavy chain variable domain and a first light chain variable domain which form a binding domain having binding specificity for a first epitope and a second heavy chain variable domain and a second light chain variable domain which form a binding domain having binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, a bispecific antibody comprises a scFv, or fragment thereof, having binding specificity for a first epitope, and a heavy chain variable domain sequence and a light chain variable domain sequence which have binding specificity for a second epitope. In preferred embodiments of the invention, the first epitope is located on LTBR and the second epitope is located on fibronectin, in particular EDB thereof.

In certain embodiments, a multispecific binding molecule according to the invention comprises an antibody, e.g. an IgG, with an scFv fused to the antibody. The scFv may in certain embodiments have binding specificity for LTBR. Both arms (comprising the variable regions) of the antibody may in certain embodiments bind to EDB of fibronectin. The scFv may be fused to a light chain of the antibody or to a heavy chain of the antibody, and may be fused to the N-terminus or to the C-terminus of the heavy or light chain. In certain embodiments, the scFv is fused to the N-terminus of the heavy chain. In other embodiments, the scFv is fused to the C-terminus of the heavy chain. It will be clear to the skilled person based on the instant disclosure that other forms are also possible, e.g. wherein a bispecific antibody comprising one arm specifically binding to LTBR and another arm specifically binding to EDB is supplemented by fusing an scFv specifically binding to EDB to one of the chains of the antibody, etc.

As used herein, the term “LTBR” refers to a polypeptide that is a cell surface receptor for lymphotoxin involved in apoptosis and cytokine release, which is a member of the tumor necrosis factor receptor superfamily. LTBR can also be referred to as “tumor necrosis factor receptor superfamily member 3 (TNFRSF3).” LTBR is expressed on the surface of many cell types, including cells of epithelial and myeloid lineages. LTBR can specifically bind the lymphotoxin membrane form (a complex of lymphotoxin-alpha and lymphotoxin-beta). Activation of LTBR can trigger apoptosis via TRAF3 and TRAF5 and can lead to the release of interleukin 8. Unless noted, preferably the LTBR is a human LTBR. A human LTBR amino acid sequence is provided by UniProt number P36941.

The term “EDB” or “extra domain B” refers to a domain of fibronectin that can be included in fibronectin molecules based on the splicing pattern of the fibronectin pre-mRNA. Extra domain B is a complete fibronectin (FN) type III repeat that comprises 91 amino acid residues. Generally, EDB is undetectable in normal adult tissues, but exhibits greater expression in fetal and tumor tissues in the extracellular matrix, and accumulates around neovasculature during angiogenic processes, thus making EDB a potential marker and target of angiogenesis. Unless noted, preferably EDB is a human EDB. A human EDB containing fibronectin isoform amino acid sequence is provided by UniProt number P02751.

The term “fibronectin” refers to a polypeptide that is a high molecular weight glycoprotein of the extracellular matrix. Fibronectin can bind to membrane-spanning receptor proteins, referred to as integrins. Fibronectin can also bind other extracellular matrix proteins, such as collagen, fibrin, and heparan sulfate proteoglycans. Fibronectin can exist as a protein dimer, consisting of two nearly identical monomers linked by a pair of disulfide bonds. Fibronectin is produced from a single gene, but alternative splicing of the fibronectin pre-mRNA molecule leads to the creation of several isoforms of fibronectin, one of which is EDB fibronectin. Fibronectin can play a role in cell adhesion, growth, migration, and differentiation, and it can be important for processes such as wound healing and embryonic development. A human fibronectin amino acid sequence is provided by UniProt number P02751, which contains extra domain B, and NCBI Accession Numbers NP_001263337 (isoform B), NP_001263338 (isoform c), NP_001263339 (isoform d), NP_001263340 (isoform e), and NP_001263341 (isoform f), NP_001293058 (isoform 8), NP_001293059 (isoform 9), NP_001293060 (isoform 10), NP_001293061 (isoform 11), and NP_002017 (isoform 3).

As used herein, an antibody or binding molecule that “specifically binds to LTBR” refers to an antibody or molecule comprising an antigen binding domain thereof that binds to a LTBR, preferably a human LTBR, with a KD of 1×10⁻⁷ M or less, preferably 1×10⁻⁸M or less, more preferably 5×10⁻⁹ M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a BIACORE® system, or by using bio-layer interferometry technology, such as an Octet RED96 system. In preferred embodiments a binding domain with specificity towards LTBR in the multispecific binding molecules of the invention comprise an antibody against LTBR, preferably an agonistic antibody against LTBR, or a functional fragment or derivative thereof, such as an scFv. An “agonistic antibody against LTBR” as used herein is an antibody binding to LTBR and capable of inducing downstream signaling either directly or upon higher order clustering, e.g. by immobilization to a solid support, by use of cross-linking antibodies, etc. Agonistic antibodies against LTBR as such have been described, and non-limiting examples are BHA10 (e.g. WO2004002431), CBE11 (e.g. WO0230986), REA412 (commercially available from Miltenyi Biotec), 31G4D8 (commercially available from BioLegend), and 71319/MAB629 (commercially available from Novus Biologicals), or can alternatively be generated according to known methods for antibody generation, such as immunization of mice, phage display, etc.

As used herein, an antigen binding domain or antigen binding fragment that “specifically binds to EDB” refers to an antigen binding domain or antigen binding fragment that binds EDB (e.g. in the form of EDB fibronectin), with a KD of 1×10⁻⁷ M or less, preferably 1×10⁻⁸M or less, more preferably 5×10⁻⁹ M or less, 1×10⁻⁹ M or less, 5×10⁻¹⁰ M or less, or 1×10⁻¹⁰ M or less.

In preferred embodiments a binding domain with specificity towards EDB in the multispecific binding molecules of the invention comprise an antibody against EDB, or a functional fragment or derivative thereof, such as an scFv. Antibodies against EDB as such have been described, and a non-limiting examples are L19 (e.g. WO9745544) and other antibodies binding to ED-B or to adjacent domains (e.g. Carnemolla et al. Int. J. Cancer: 68, 397-405 (1996)), or can alternatively be generated according to known methods for antibody generation, such as immunization of mice, phage display, etc.

The smaller the value of the KD of an antibody, the higher affinity that the antibody binds to a target antigen.

According to a particular aspect of the invention, provided herein are multispecific binding molecules. The multispecific binding molecules comprise (i) a first binding domain that specifically binds to a lymphotoxin beta receptor (LTBR), and (ii) a second binding domain that specifically binds to EDB, wherein the multispecific binding molecule activates LTBR upon binding of the EDB.

In certain embodiments, the multispecific binding molecule activates LTBR in a tumor specific manner. Activating LTBR in a tumor specific manner, as used herein, means that the upon simultaneous binding of the multispecific binding molecule to LTBR and the EDB, which are both present in the tumor microenvironment either on the surface of a cell or present in the extracellular matrix, LTBR is activated to trigger signaling via canonical and/or non-canonical NF-κB pathway. Activation of NF-κB pathways can lead to the establishment of a pro-inflammatory tumor microenvironment via secretion of pro-inflammatory chemokines and cytokines and expression of adhesion molecules on the surface of the cell. Simultaneous binding of the multispecific binding molecule results in activation of LTBR in the tumor. If the EDB is not present in normal tissue, i.e., normal cells, or not present in the extracellular matrix adjacent to normal tissue, the multispecific binding molecule can on normal tissue only bind LTBR, which will not result in the activation of LTBR in normal tissue. This is a significant advantage over molecules described in the prior art that are based on natural LTBR ligands, e.g. LIGHT-antibody fusions, which can activate LTBR independent of a TAA, and thus are much less tumor specific for activation of LTBR as compared to the molecules of the invention, as shown in the examples herein.

In certain embodiments, the multispecific binding molecule comprises two binding domains, such as for instance a bispecific antibody that comprises two antigen binding domains, one binding to LTBR and another one binding to EDB. In preferred embodiments, the multispecific binding molecule comprises more than two antigen binding domains, for instance one binding to LTBR and two binding to the EDB. In certain embodiments, the multispecific binding molecule comprises three binding domains. In certain embodiments, the three binding domains are all different and bind to three different antigens. In certain preferred embodiments, the three antigen binding domains comprise one binding domain that binds to a first antigen, and two binding domains that bind to a second antigen. In this embodiment, the three antigen binding domains are present in a 2:1 stoichiometry. The three antigen binding domains can, for example, comprise one first binding domain that specifically binds to an LTBR on an LTBR-expressing cell. The three antigen binding domains can, for example, comprise two second binding domains that specifically bind EDB. In certain embodiments, the two second binding domains have identical binding specificity for the EDB, e.g. the two second binding domains may be identical. It is shown herein that multispecific binding molecules of the invention that have more than one binding domain specific for EDB have further advantageous properties over multispecific binding molecules of the invention that have only one binding domain specific for EDB. In certain embodiments, LTBR is activated upon binding of LTBR and the EDB (which EDB is part of fibronectin that is present in the extracellular matrix in tumor tissue).

According to a particular aspect, provided herein are isolated anti-lymphotoxin beta receptor (LTBR) bispecific antibodies or antigen binding fragments thereof. In certain non-limiting embodiments, the binding domain that specifically binds to LTBR comprises an agonistic anti-LTBR antibody or a fragment or derivative thereof, for instance a single chain antibody fragment (scFv), comprising a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said VH and VL comprise any of the following:

(i) HCDR1, HCDR2 and HCDR3 comprising the amino acid sequences of SEQ ID NO:60, SEQ ID NO:61, and SEQ ID NO:62, respectively, and LCDR1, LCDR2 and LCDR3 comprising the amino acid sequences of SEQ ID NO:63, SEQ ID NO:64, and SEQ ID NO:65, respectively; or
(ii) HCDR1, HCDR2 and HCDR3 comprising the amino acid sequences of SEQ ID NO:83, SEQ ID NO:61, and SEQ ID NO:62, respectively, and LCDR1, LCDR2 and LCDR3 comprising the amino acid sequences of SEQ ID NO:63, SEQ ID NO:64, and SEQ ID NO:65, respectively; or
(iii) HCDR1, HCDR2 and HCDR3 comprising the amino acid sequences of SEQ ID NO:66, SEQ ID NO:67, and SEQ ID NO:68, respectively, and LCDR1, LCDR2, and LCDR3 comprising the amino acid sequences of SEQ ID NO:69, SEQ ID NO:70, and SEQ ID NO:71, respectively; or
(iv) VH comprises an amino acid sequence having at least 95%, 96%, 97%, 98%, 99% identity or 100% identity to the amino acid sequence of SEQ ID NO:43, and VL comprises an amino acid sequence having at least 95%, 96%, 97%, 98%, 99% identity or 100% identity to the amino acid sequence of SEQ ID NO:44, such as wherein VH comprises an amino acid sequence having at least 95% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 96% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 97% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 98% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 99% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having 100% identity to the amino acid sequence of SEQ ID NO:43, and VL comprises an amino acid sequence having at least 95%, 96%, 97%, 98%, 99% identity or 100% identity to the amino acid sequence of SEQ ID NO:44; or
(v) VH comprises an amino acid sequence having at least 95%, 96%, 97%, 98%, 99% identity or 100% identity to the amino acid sequence of SEQ ID NO:47, and VL comprises an amino acid sequence having at least 95%, 96%, 97%, 98%, 99% identity or 100% identity to the amino acid sequence of SEQ ID NO:48, such as wherein VH comprises an amino acid sequence having at least 95% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 96% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 97% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 98% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 99% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having 100% identity to the amino acid sequence of SEQ ID NO:47, and VL comprises an amino acid sequence having at least 95%, 96%, 97%, 98%, 99% identity or 100% identity to the amino acid sequence of SEQ ID NO:48: or

### (vi) SEQ ID NO: 22; or

(vii) SEQ ID NO: 23; or
(viii) SEQ ID NO: 25.

In certain non-limiting embodiments, the second binding domain that specifically binds to EDB comprises an antibody binding to EDB or a fragment or derivative of such antibody, for instance comprising a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said antibody or fragment thereof comprise any of the following:

(i) HCDR1, HCDR2 and HCDR3 comprising the amino acid sequences of SEQ ID NO:72, SEQ ID NO:73, and SEQ ID NO:74, respectively, and LCDR1, LCDR2 and LCDR3 comprising the amino acid sequences of SEQ ID NO:75, SEQ ID NO:76, and SEQ ID NO:77, respectively; or
(ii) VH comprises an amino acid sequence having at least 95%, 96%, 97%, 98%, 99% identity or 100% identity to the amino acid sequence of SEQ ID NO:45, and VL comprises an amino acid sequence having at least 95%, 96%, 97%, 98%, 99% identity or 100% identity to the amino acid sequence of SEQ ID NO:46, such as wherein VH comprises an amino acid sequence having at least 95% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 96% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 97% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 98% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 99% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having 100% identity to the amino acid sequence of SEQ ID NO:45, and VL comprises an amino acid sequence having at least 95%, 96%, 97%, 98%, 99% identity or 100% identity to the amino acid sequence of SEQ ID NO:46.

In certain non-limiting embodiments, the multispecific binding molecule comprises:

(1) a binding domain that specifically binds to LTBR comprises a BHA10 antibody or of a CBE11 antibody or a fragment or derivative thereof, for instance a single chain antibody fragment (scFv), comprising a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said VH and VL comprise any of the following:
(i) HCDR1, HCDR2 and HCDR3 comprising the amino acid sequences of SEQ ID NO:60, SEQ ID NO:61, and SEQ ID NO:62, respectively, and LCDR1, LCDR2 and LCDR3 comprising the amino acid sequences of SEQ ID NO:63, SEQ ID NO:64, and SEQ ID NO:65, respectively; or
(ii) HCDR1, HCDR2 and HCDR3 comprising the amino acid sequences of SEQ ID NO:83, SEQ ID NO:61, and SEQ ID NO:62, respectively, and LCDR1, LCDR2 and LCDR3 comprising the amino acid sequences of SEQ ID NO:63, SEQ ID NO:64, and SEQ ID NO:65, respectively; or
(iii) HCDR1, HCDR2 and HCDR3 comprising the amino acid sequences of SEQ ID NO:66, SEQ ID NO:67, and SEQ ID NO:68, respectively, and LCDR1, LCDR2, and LCDR3 comprising the amino acid sequences of SEQ ID NO:69, SEQ ID NO:70, and SEQ ID NO:71, respectively; or
(iv) VH comprises an amino acid sequence having at least 95%, 96%, 97%, 98%, 99% identity or 100% identity to the amino acid sequence of SEQ ID NO:43, and VL comprises an amino acid sequence having at least 95%, 96%, 97%, 98%, 99% identity or 100% identity to the amino acid sequence of SEQ ID NO:44, such as wherein VH comprises an amino acid sequence having at least 95% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 96% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 97% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 98% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 99% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having 100% identity to the amino acid sequence of SEQ ID NO:43, and VL comprises an amino acid sequence having at least 95%, 96%, 97%, 98%, 99% identity or 100% identity to the amino acid sequence of SEQ ID NO:44; or
(v) VH comprises an amino acid sequence having at least 95%, 96%, 97%, 98%, 99% identity or 100% identity to the amino acid sequence of SEQ ID NO:47, and VL comprises an amino acid sequence having at least 95%, 96%, 97%, 98%, 99% identity or 100% identity to the amino acid sequence of SEQ ID NO:48, such as wherein VH comprises an amino acid sequence having at least 95% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 96% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 97% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 98% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 99% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having 100% identity to the amino acid sequence of SEQ ID NO:47, and VL comprises an amino acid sequence having at least 95%, 96%, 97%, 98%, 99% identity or 100% identity to the amino acid sequence of SEQ ID NO:48; or

### (vi) SEQ ID NO: 22; or

(vii) SEQ ID NO: 23; or
(viii) SEQ ID NO: 25; and
(2) a second binding domain that specifically binds to EDB comprises an L19 antibody or a fragment or derivative thereof, for instance comprising a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said antibody or fragment thereof comprise HCDR1, HCDR2 and HCDR3 comprising the amino acid sequences of SEQ ID NO:72, SEQ ID NO:73, and SEQ ID NO:74, respectively, and LCDR1, LCDR2 and LCDR3 comprising the amino acid sequences of SEQ ID NO:75, SEQ ID NO:76, and SEQ ID NO:77, respectively.

In certain non-limiting embodiments, the multispecific binding molecule comprises:

(1) a binding domain that specifically binds to LTBR comprises a BHA10 antibody or of a CBE11 antibody or a fragment or derivative thereof, for instance a single chain antibody fragment (scFv), comprising a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said VH and VL comprise any of the following:
(i) HCDR1, HCDR2 and HCDR3 comprising the amino acid sequences of SEQ ID NO:60, SEQ ID NO:61, and SEQ ID NO:62, respectively, and LCDR1, LCDR2 and LCDR3 comprising the amino acid sequences of SEQ ID NO:63, SEQ ID NO:64, and SEQ ID NO:65, respectively; or
(ii) HCDR1, HCDR2 and HCDR3 comprising the amino acid sequences of SEQ ID NO:83, SEQ ID NO:61, and SEQ ID NO:62, respectively, and LCDR1, LCDR2 and LCDR3 comprising the amino acid sequences of SEQ ID NO:63, SEQ ID NO:64, and SEQ ID NO:65, respectively; or
(iii) HCDR1, HCDR2 and HCDR3 comprising the amino acid sequences of SEQ ID NO:66, SEQ ID NO:67, and SEQ ID NO:68, respectively, and LCDR1, LCDR2, and LCDR3 comprising the amino acid sequences of SEQ ID NO:69, SEQ ID NO:70, and SEQ ID NO:71, respectively; or
(iv) VH comprises an amino acid sequence having at least 95%, 96%, 97%, 98%, 99% identity or 100% identity to the amino acid sequence of SEQ ID NO:43, and VL comprises an amino acid sequence having at least 95%, 96%, 97%, 98%, 99% identity or 100% identity to the amino acid sequence of SEQ ID NO:44, such as wherein VH comprises an amino acid sequence having at least 95% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 96% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 97% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 98% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 99% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having 100% identity to the amino acid sequence of SEQ ID NO:43, and VL comprises an amino acid sequence having at least 95%, 96%, 97%, 98%, 99% identity or 100% identity to the amino acid sequence of SEQ ID NO:44; or
(v) VH comprises an amino acid sequence having at least 95%, 96%, 97%, 98%, 99% identity or 100% identity to the amino acid sequence of SEQ ID NO:47, and VL comprises an amino acid sequence having at least 95%, 96%, 97%, 98%, 99% identity or 100% identity to the amino acid sequence of SEQ ID NO:48, such as wherein VH comprises an amino acid sequence having at least 95% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 96% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 97% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 98% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 99% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having 100% identity to the amino acid sequence of SEQ ID NO:47, and VL comprises an amino acid sequence having at least 95%, 96%, 97%, 98%, 99% identity or 100% identity to the amino acid sequence of SEQ ID NO:48; or

### (vi) SEQ ID NO: 22; or

(vii) SEQ ID NO: 23; or
(viii) SEQ ID NO: 25; and
(2) a second binding domain that specifically binds to EDB comprises an L19 antibody or a fragment or derivative thereof, for instance comprising a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said antibody or fragment thereof comprises a VH that comprises an amino acid sequence having at least 95%, 96%, 97%, 98%, 99% identity or 100% identity to the amino acid sequence of SEQ ID NO:45, and VL comprises an amino acid sequence having at least 95%, 96%, 97%, 98%, 99% identity or 100% identity to the amino acid sequence of SEQ ID NO:46; such as wherein VH comprises an amino acid sequence having at least 95% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 96% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 97% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 98% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 99% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having 100% identity to the amino acid sequence of SEQ ID NO:45, and VL comprises an amino acid sequence having at least 95%, 96%, 97%, 98%, 99% identity or 100% identity to the amino acid sequence of SEQ ID NO:46.

In certain non-limiting embodiments, the multispecific molecule comprises:

(1) a binding domain that specifically binds to LTBR comprising SEQ ID NO: 22; and
(2) a second binding domain that specifically binds to EDB comprises an L19 antibody or a fragment or derivative thereof, for instance comprising a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said antibody or fragment thereof comprise any of the following:
(i) HCDR1, HCDR2 and HCDR3 comprising the amino acid sequences of SEQ ID NO:72, SEQ ID NO:73, and SEQ ID NO:74, respectively, and LCDR1, LCDR2 and LCDR3 comprising the amino acid sequences of SEQ ID NO:75, SEQ ID NO:76, and SEQ ID NO:77, respectively; or
(ii) VH comprises an amino acid sequence having at least 95%, 96%, 97%, 98%, 99% identity or 100% identity to the amino acid sequence of SEQ ID NO:45, and VL comprises an amino acid sequence having at least 95%, 96%, 97%, 98%, 99% identity or 100% identity to the amino acid sequence of SEQ ID NO:46.

In certain non-limiting embodiments, the multispecific molecule comprises:

(1) a binding domain that specifically binds to LTBR comprising SEQ ID NO: 23; and
(2) a second binding domain that specifically binds to EDB comprises an L19 antibody or a fragment or derivative thereof, for instance comprising a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said antibody or fragment thereof comprise any of the following:
(i) HCDR1, HCDR2 and HCDR3 comprising the amino acid sequences of SEQ ID NO:72, SEQ ID NO:73, and SEQ ID NO:74, respectively, and LCDR1, LCDR2 and LCDR3 comprising the amino acid sequences of SEQ ID NO:75, SEQ ID NO:76, and SEQ ID NO:77, respectively; or
(ii) VH comprises an amino acid sequence having at least 95%, 96%, 97%, 98%, 99% identity or 100% identity to the amino acid sequence of SEQ ID NO:45, and VL comprises an amino acid sequence having at least 95%, 96%, 97%, 98%, 99% identity or 100% identity to the amino acid sequence of SEQ ID NO:46.
In certain non-limiting embodiments, the multispecific molecule comprises:
(1) a binding domain that specifically binds to LTBR comprising SEQ ID NO: 25; and
(2) a second binding domain that specifically binds to EDB comprises an L19 antibody or a fragment or derivative thereof, for instance comprising a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said antibody or fragment thereof comprise any of the following:
(i) HCDR1, HCDR2 and HCDR3 comprising the amino acid sequences of SEQ ID NO:72, SEQ ID NO:73, and SEQ ID NO:74, respectively, and LCDR1, LCDR2 and LCDR3 comprising the amino acid sequences of SEQ ID NO:75, SEQ ID NO:76, and SEQ ID NO:77, respectively; or
(ii) VH comprises an amino acid sequence having at least 95%, 96%, 97%, 98%, 99% identity or 100% identity to the amino acid sequence of SEQ ID NO:45, and VL comprises an amino acid sequence having at least 95%, 96%, 97%, 98%, 99% identity or 100% identity to the amino acid sequence of SEQ ID NO:46.

In certain non-limiting embodiments, the multispecific binding molecule comprises any of the following:

(a) (i) a first heavy chain comprising the amino acid sequence of SEQ ID NO: 1 forming a binding domain with a first light chain comprising the amino acid sequence of SEQ ID NO: 2, and (ii) a second heavy chain comprising the amino acid sequence of SEQ ID NO: 4 forming a binding domain with a second light chain comprising the amino acid sequence of SEQ ID NO: 5 [multispecific binding molecule referred to as COVA14121]; or
(b) (i) a first heavy chain comprising the amino acid sequence of SEQ ID NO: 9 forming a binding domain with a first light chain comprising the amino acid sequence of SEQ ID NO: 10, and (ii) a second heavy chain comprising the amino acid sequence of SEQ ID NO: 4 forming a binding domain with a second light chain comprising the amino acid sequence of SEQ ID NO: 5 [multi specific binding molecule referred to as COVA14122].

In certain further non-limiting embodiments, the multispecific binding molecule comprises any of the following:

(c) (i) an scFv-heavy chain fusion comprising the amino acid sequence of SEQ ID NO: 30, the heavy chain part thereof (comprising SEQ ID NO: 84) forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 [multispecific binding molecule referred to as COVA1480]; or
(d) (i) an scFv-heavy chain fusion comprising the amino acid sequence of SEQ ID NO: 31, the heavy chain part thereof (comprising SEQ ID NO: 84) forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 [multispecific binding molecule referred to as COVA1481]; or
(e) (i) an scFv-heavy chain fusion comprising the amino acid sequence of SEQ ID NO: 32, the heavy chain part thereof (comprising SEQ ID NO: 84) forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 [multispecific binding molecule referred to as COVA1482]; or
(f) (i) an scFv-heavy chain fusion comprising the amino acid sequence of SEQ ID NO: 33, the heavy chain part thereof (comprising SEQ ID NO: 84) forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 [multispecific binding molecule referred to as COVA1483]; or
(g) (i) an scFv-heavy chain fusion comprising the amino acid sequence of SEQ ID NO: 34, the heavy chain part thereof (comprising SEQ ID NO: 84) forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 [multispecific binding molecule referred to as COVA14107]; or
(h) (i) an scFv-heavy chain fusion comprising the amino acid sequence of SEQ ID NO: 35, the heavy chain part thereof (comprising SEQ ID NO: 84) forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 [multispecific binding molecule referred to as COVA14108]; or
(j) (i) an scFv-heavy chain fusion comprising the amino acid sequence of SEQ ID NO: 38, the heavy chain part thereof (comprising SEQ ID NO: 3) forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 [multispecific binding molecule referred to as COVA14133]; or
(k) (i) an scFv-heavy chain fusion comprising the amino acid sequence of SEQ ID NO: 39, the heavy chain part thereof (comprising SEQ ID NO: 3) forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 [multispecific binding molecule referred to as COVA14174]; or
(l) (i) an scFv-heavy chain fusion comprising the amino acid sequence of SEQ ID NO: 56, the heavy chain part thereof (comprising SEQ ID NO: 84) forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 [multispecific binding molecule referred to as COVA1456].

In some embodiments, the multispecific molecule comprises (i) an scFv-heavy chain fusion comprising the amino acid sequence of SEQ ID NO: 38, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5.

In some embodiments, the multispecific (e.g. bispecific) molecule induces NF-κB signaling in the presence of EDB that is at least 2-fold, such as at least 3-fold, for example at least 4-fold greater than the NF-κB signaling induced in the absence of EDB (under the same conditions). Sometimes the assay is an NF-κB luciferase reporter assay. The NF-κB luciferase reporter assay may be performed using the protocol of Example 2.

In some embodiments, the multispecific (e.g. bispecific) molecule induces ICAM-1 expression of the surface of cells in the presence of EDB that is at least 2-fold, such as at least 3-fold, for example at least 4-fold greater than the ICAM-1 expression induced in the absence of EDB (under the same conditions). Sometimes the assay is an in vitro LTBR activation assay, such as an A375/WI38A subline2RA co-culture cell assay. The A375/WI38A subline2RA co-culture cell assay may be performed using the protocol of Example 3.

According to a particular aspect, the heavy chains and light chains are humanized.

In some embodiments, the bispecific antibody of the present invention comprises a diabody, a cross-body, an scFv, a Duobody, an spFv, or a bispecific antibody obtained via a controlled Fab arm exchange, as those described in the present invention.

In some embodiments, the bispecific antibodies include IgG-like molecules with complementary CH3 domains to force heterodimerization; recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

In some embodiments, IgG-like molecules with complementary CH3 domains molecules include the Triomab/Quadroma (Trion Pharma/Fresenius Biotech), the Knobs-into-Holes (Genentech), CrossMAbs (Roche) and the electrostatically-matched (Amgen), the LUZ-Y (Genentech), the Strand Exchange Engineered Domain body (SEEDbody) (EMD Serono), the Biclonic (Merus), or the DuoBody (Genmab A/S).

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) or CovX-body (CovX/Pfizer).

In some embodiments, IgG fusion molecules include Dual Variable Domain (DVD)-Ig (Abbott), IgG-like Bispecific (InnClone/Eli Lilly), Ts2Ab (MedImmune/AZ) and BsAb (Zymogenetics), HERCULES (Biogen Idec), or TvAb (Roche).

In some embodiments, Fc fusion molecules can include ScFv/Fc Fusions (Academic Institution), SCORPION (Emergent BioSolutions/Trubion, Zymogenetics/BMS), Dual Affinity Retargeting Technology (Fc-DART) (MacroGenics), or Dual(ScFv)₂-Fab (National Research Center for Antibody Medicine—China).

In some embodiments, Fab fusion bispecific antibodies include F(ab)₂ (Medarex/AMGEN), Dual-Action or Bis-Fab (Genentech), Dock-and-Lock (DNL) (ImmunoMedics), Bivalent Bispecific (Biotecnol), or Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack), or COMBODY (Epigen Biotech), dual targeting nanobodies (Ablynx), dual targeting heavy chain only domain antibodies.

Full length bispecific antibodies of the invention can be generated for example using Fab arm exchange (or half molecule exchange) between two mono specific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent mono specific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms can be engineered to favor heterodimerization over homodimerization. The resulting product is a bispecific antibody having two Fab arms or half molecules which each binding a distinct epitope, i.e. an epitope on LTBR and an epitope on EDB of fibronectin.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Publ. No. WO2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob.” Exemplary CH3 substitution pairs forming a knob and a hole are (expressed as modified position in the first CH3 domain of the first heavy chain/modified position in the second CH3 domain of the second heavy chain, using Kabat numbering): T366Y/F405A, T366W/F405W, F405W/Y407A, T394W/Y407T, T394S/Y407A, T366W/T394S, F405W/T394S, or T366W/T366S_L368A_Y407V.

Other strategies such as promoting heavy chain heterodimerization using electrostatic interactions by substituting positively charged residues at one CH3 surface and negatively charged residues at a second CH3 surface can be used, as described for instance in US Pat. Publ. No. US2010/0015133; US Pat. Publ. No. US2009/0182127; US Pat. Publ. No. US2010/028637; or US Pat. Publ. No. US2011/0123532. In other strategies, heterodimerization can be promoted by the following substitutions (expressed as modified position in the first CH3 domain of the first heavy chain/modified position in the second CH3 domain of the second heavy chain): L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V K409F Y407A/T366A_K409F, or T350V_L351Y_F405A Y407V/T350V_T366L_K392L_T394W e.g. as described in U.S. Pat. Publ. No. US2012/0149876 or U.S. Pat. Publ. No. US2013/0195849.

In addition to methods described above, bispecific antibodies of the invention can be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in PCT Pat. Publ. No. WO2011/131746. In the methods, the first monospecific bivalent antibody and the second monospecific bivalent antibody are engineered to have certain substitutions at the CH3 domain that promotes heterodimer stability; the antibodies are incubated together under reducing conditions sufficient to allow the cysteines in the hinge region to undergo disulfide bond isomerization; thereby generating the bispecific antibody by Fab arm exchange. The incubation conditions can optionally be restored to non-reducing conditions. Exemplary reducing agents that can be used are 2-mercaptoethylamine (2-MEA), dithiothreitol (DTT), dithioerythritol (DTE), glutathione, tris (2-carboxyethyl) phosphine (TCEP), L-cysteine and beta-mercaptoethanol, preferably a reducing agent selected from the group consisting of: 2-mercaptoethylamine, dithiothreitol and tris (2-carboxyethyl) phosphine. For example, incubation for at least 90 min at a temperature of at least 20° C. in the presence of at least 25 mM 2-MEA or in the presence of at least 0.5 mM dithiothreitol at a pH from 5-8, for example at pH of 7.0 or at pH of 7.4 can be used.

In some embodiments described herein, immune effector properties of the multispecific binding molecules such as bispecific antibodies of the invention can be modified, preferably silenced, e.g. through Fc modifications by techniques known to those skilled in the art. For example, Fc effector functions such as C1q binding, complement dependent cytotoxicity (CDC), antibody-dependent cell-mediated cytotoxicity (ADCC), antibody-dependent cell-mediated phagocytosis (ADCP), down regulation of cell surface receptors (e.g., B cell receptor; BCR), etc. can be provided and/or controlled by modifying residues in the Fc responsible for these activities, see, e.g., N297 mutations in Nose et al., PNAS (1983); LALA mutations in Xu et al., Cell Immunol. 200(1):16-26) (2000); and DANA mutations in Wilson et al., Cancer Cell 19(1):101-113 (2011); or e.g. mutations of aspartic acid (D) at position 265, asparagine (N) at position 297 and proline (P) at position 329, wherein numbering is indicated by the EU index as in Kabat, e.g. each to alanine (A) to get a so-called DANAPA mutant, as described in detail in WO 2019/068632.

“Antibody-dependent cell-mediated cytotoxicity” or “ADCC” refers to a cell-mediated reaction in which non-specific cytotoxic cells that express Fc receptors (FcRs) (e.g. Natural Killer (NK) cells, neutrophils, and macrophages) recognize bound antibody on a target cell and subsequently cause lysis of the target cell.

In certain embodiments, the multispecific binding molecule of the invention comprises a bispecific antibody that is chimeric.

In certain embodiments, the multispecific binding molecule of the invention comprises a bispecific antibody that is human or humanized.

In another general aspect, the invention relates to one or more nucleic acids encoding a multispecific binding molecule, e.g. bispecific antibody or antigen-binding fragment thereof of the invention. By way of a non-limiting example, a heavy chain for a bispecific antibody can be encoded by one nucleic acid, and a light chain can be encoded by a second nucleic acid. In another example, a heavy chain and a light chain of a bispecific antibody may be encoded on a single nucleic acid molecule. It will be appreciated by those skilled in the art that the coding sequence of a protein can be changed (e.g., replaced, deleted, inserted, etc.) without changing the amino acid sequence of the protein in view of the degeneracy of the genetic code. Accordingly, it will be understood by those skilled in the art that nucleic acid sequences encoding monoclonal antibodies and/or bispecific antibodies of the invention can be altered without changing the amino acid sequences of the proteins. Additionally, the one or more nucleic acids of the invention can be isolated nucleic acids. Accordingly, the invention relates to any nucleic acid molecule or combination of nucleic acid molecules encoding a molecule of the invention.

In another general aspect, the invention relates to one or more vectors comprising the one or more nucleic acids of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and/or origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments of the invention. Such techniques are well known to those skilled in the art in view of the present disclosure.

In another general aspect, the invention relates to a host cell comprising the one or more vectors comprising the one or more nucleic acids encoding a multispecific binding molecule such as a bispecific antibody or an antigen-binding fragment thereof of the invention. Any host cell known to those skilled in the art in view of the present disclosure can be used for recombinant expression of multispecific binding molecules such as bispecific antibodies or antigen-binding fragments thereof of the invention. In some embodiments, the host cells are E. coli TG1 or BL21 cells (for expression of, e.g., an scFv or Fab antibody), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it can be stably integrated into the host cell genome such that the recombinant nucleic acid is effectively expressed.

In another general aspect, the invention relates to a method of producing a multispecific binding molecule such as a bispecific antibody or antigen-binding fragment thereof disclosed herein. The methods comprise culturing a cell comprising a nucleic acid encoding the multispecific binding molecule such as a bispecific antibody or antigen-binding fragment thereof under conditions to produce a multispecific binding molecule such as a bispecific antibody or antigen-binding fragment thereof disclosed herein and recovering the multispecific binding molecule such as a bispecific antibody or antigen-binding fragment thereof from the cell or cell culture (e.g., from the supernatant). Expressed multispecific binding molecule such as bispecific antibodies or antigen-binding fragments thereof can be harvested from the cells and purified according to conventional techniques known in the art and as described herein.

Pharmaceutical Compositions

In another general aspect, the invention relates to a pharmaceutical composition comprising a multispecific binding molecule (e.g., a bispecific antibody or antigen-binding fragment thereof) of the invention and a pharmaceutically acceptable carrier. The term “pharmaceutical composition” as used herein means a product comprising a multispecific binding molecule of the invention together with a pharmaceutically acceptable carrier. Multispecific binding molecules (e.g., bispecific antibodies) of the invention and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in an antibody pharmaceutical composition can be used herein.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carriers can be used in formulating the pharmaceutical compositions of the invention.

In one embodiment of the invention, the pharmaceutical composition is a liquid formulation. A preferred example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms can be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract or under the skin.

In other embodiments, the pharmaceutical composition can be delivered intranasally, intrabuccally, or sublingually.

The pH in an aqueous formulation can be between pH 3 and pH 10. In one embodiment of the invention, the pH of the formulation is from about 7.0 to about 9.5. In another embodiment of the invention, the pH of the formulation is from about 3.0 to about 7.0.

In certain embodiments, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, or tris(hydroxymethyl)-aminomethane, and mixtures thereof. The buffer can be present individually or in the aggregate, in a concentration from about 0.01 mg/ml to about 50 mg/ml, for example from about 0.1 mg/ml to about 20 mg/ml. Pharmaceutical compositions comprising each one of these specific buffers constitute alternative embodiments of the invention.

In certain embodiments, the pharmaceutical composition comprises a preservative. Non-limiting examples of preservatives include: benzethonium chloride, benzoic acid, benzyl alcohol, bronopol, butyl 4-hydroxybenzoate, chlorobutanol, chlorocresol, chlorohexidine, chlorphenesin, o-cresol, m-cresol, p-cresol, ethyl 4-hydroxybenzoate, imidurea, methyl 4-hydroxybenzoate, phenol, 2-phenoxyethanol, 2-phenylethanol, propyl 4-hydroxybenzoate, sodium dehydroacetate, thiomerosal, and mixtures thereof. The preservative can be present individually or in the aggregate, in a concentration from about 0.01 mg/ml to about 50 mg/ml, for example from about 0.1 mg/ml to about 20 mg/ml. Pharmaceutical compositions comprising each one of these specific preservatives constitute alternative embodiments of the invention.

In certain embodiments, the pharmaceutical composition comprises an isotonic agent. Non-limiting examples of isotonic agents include a salt (such as sodium chloride), an amino acid (such as glycine, histidine, arginine, lysine, isoleucine, aspartic acid, tryptophan, or threonine), an alditol (such as glycerol, 1,2-propanediol propyleneglycol), 1,3-propanediol, or 1,3-butanediol), polyethyleneglycol (e.g. PEG400), and mixtures thereof. Another example of an isotonic agent includes a sugar. Non-limiting examples of sugars can include mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alpha and beta-HPCD, soluble starch, hydroxyethyl starch, or sodium carboxymethyl-cellulose. Another example of an isotonic agent is a sugar alcohol, wherein the term “sugar alcohol” is defined as a C(4-8) hydrocarbon having at least one —OH group. Non-limiting examples of sugar alcohols include mannitol, sorbitol, inositol, galactitol, dulcitol, xylitol, or arabitol. The isotonic agent can be present individually or in the aggregate, in a concentration from about 0.01 mg/ml to about 50 mg/ml, for example from about 0.1 mg/ml to about 20 mg/ml. Pharmaceutical compositions comprising each one of these specific isotonic agents constitute alternative embodiments of the invention.

In certain embodiments, the pharmaceutical composition comprises a chelating agent. Non-limiting examples of chelating agents include citric acid, aspartic acid, salts of ethylenediaminetetraacetic acid (EDTA), and mixtures thereof. The chelating agent can be present individually or in the aggregate, in a concentration from about 0.01 mg/ml to about 50 mg/ml, for example from about 0.1 mg/ml to about 20 mg/ml. Pharmaceutical compositions comprising each one of these specific chelating agents constitute alternative embodiments of the invention.

In certain embodiments, the pharmaceutical composition comprises a stabilizer. Non-limiting examples of stabilizers include one or more aggregation inhibitors, one or more oxidation inhibitors, one or more surfactants, and/or one or more protease inhibitors.

In certain embodiments, the pharmaceutical composition comprises a stabilizer, wherein said stabilizer is carboxy-/hydroxycellulose and derivates thereof (such as HPC, HPC-SL, HPC-L and HPMC), cyclodextrins, 2-methylthioethanol, polyethylene glycol (such as PEG 3350), polyvinyl alcohol (PVA), polyvinyl pyrrolidone, salts (such as sodium chloride), sulphur-containing substances such as monothioglycerol), or thioglycolic acid. The stabilizer can be present individually or in the aggregate, in a concentration from about 0.01 mg/ml to about 50 mg/ml, for example from about 0.1 mg/ml to about 20 mg/ml. Pharmaceutical compositions comprising each one of these specific stabilizers constitute alternative embodiments of the invention.

In certain embodiments, the pharmaceutical composition comprises one or more surfactants. The term “surfactant” refers to any molecules or ions that are comprised of a water-soluble (hydrophilic) part, and a fat-soluble (lipophilic) part. The surfactant can, for example, be selected from the group consisting of anionic surfactants, cationic surfactants, nonionic surfactants, and/or zwitterionic surfactants. The surfactant can be present individually or in the aggregate, in a concentration from about 0.1 mg/ml to about 20 mg/ml. Pharmaceutical compositions comprising each one of these specific surfactants constitute alternative embodiments of the invention.

In certain embodiments, the pharmaceutical composition comprises one or more protease inhibitors, such as, e.g., EDTA, and/or benzamidine hydrochloric acid (HCl). The protease inhibitor can be present individually or in the aggregate, in a concentration from about 0.1 mg/ml to about 20 mg/ml. Pharmaceutical compositions comprising each one of these specific protease inhibitors constitute alternative embodiments of the invention.

In another general aspect, the invention relates to a method of producing a pharmaceutical composition comprising a multispecific binding molecule such as a bispecific antibody or antigen-binding fragment thereof of the invention, comprising combining a multispecific binding molecule such as a bispecific antibody or antigen-binding fragment thereof with a pharmaceutically acceptable carrier to obtain the pharmaceutical composition.

Methods of Use

In another general aspect, the invention relates to a method of targeting LTBR on cells present in tumors (e.g., tumor cells, fibroblasts, monocytes, etc.), the method comprising exposing the cells present in tumors to a multispecific binding molecule or a pharmaceutical composition of the invention.

The functional activity of multispecific binding molecules (e.g., bispecific antibodies and antigen-binding fragments thereof) that bind LTBR and/or EDB can be characterized by methods known in the art and as described herein. Methods for characterizing the multispecific binding molecules that bind LTBR and/or EDB include, but are not limited to, affinity and specificity assays including Biacore, ELISA, and/or OctetRed analysis; binding assays to detect the binding of the multispecific binding molecules to LTBR on cancer cells and other cell types by FACS. According to particular embodiments, the methods for characterizing multispecific binding molecules that bind LTBR and/or EDB include those described below.

In another general aspect, the invention relates to a method to establish a pro-inflammatory tumor microenvironment. The methods comprise contacting the LTBR-expressing cells in the tumor microenvironment with a multispecific binding molecule of the invention, wherein contacting the LTBR-expressing cells with the multispecific binding molecule leads to the secretion of pro-inflammatory chemokines and cytokines and expression of adhesion molecules on the cell surface.

In another general aspect, the invention relates to a method of treating a cancer in a subject in need thereof, comprising administering to the subject a multispecific binding molecule of the invention (e.g., a bispecific antibody or antigen binding fragment thereof) that specifically binds LTBR and EDB of fibronectin, or a pharmaceutical composition disclosed herein. The cancer preferably is an EDB-expressing cancer. The cancer can, for example, be an LTBR-expressing cancer. The cancer can, for example, be selected from the group consisting of a prostate cancer, a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

According to embodiments of the invention, the pharmaceutical composition comprises an effective amount of an anti-LTBR multispecific binding molecule (e.g., an anti-LTBR/anti-EDB bispecific antibody or antigen-binding fragment thereof). As used herein, the term “effective amount” refers to an amount of an active ingredient or component that elicits the desired biological or medicinal response in a subject.

According to particular embodiments, an effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

In some embodiments, the effective amount of multispecific binding molecule of the invention may be administered at a dose in the range from about 0.1 mg/kg to about 25 mg/kg, about 0.1 mg/kg to about 20 mg/kg, about 0.1 mg/kg to about 15 mg/kg, about 0.1 mg/kg to about 10 mg/kg, or about 0.1 mg/kg to about 5 mg/kg.

The effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optionally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration. In some embodiments, the compositions disclosed herein may be administered to a subject by a variety of routes such as topical, oral or parenterally. Methods of parenteral delivery include intra-arterial (directly to the tissue), intramedullary, intrathecal, intraventricular, intraperitoneal, or intranasal administration.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

According to particular embodiments, provided are compositions used in the treatment of a cancer. For cancer therapy, the compositions can be used in combination with another treatment including, but not limited to, a chemotherapy, an anti-CD20 mAb, an anti-TIM-3 mAb, an anti-CTLA-4 antibody, an anti-PD-L1 antibody, an anti-PD-1 antibody, a PD-1/PD-L1 therapy, Indoleamine-pyrrole 2,3-dioxygenase (DO), an anti-OX40 antibody, an anti-GITR antibody, an anti-CD40 antibody, an anti-CD38 antibody, cytokines, oncolytic viruses, TLR agonists, STING agonist, other immuno-oncology drugs, an antiangiogenic agent, a radiation therapy, an antibody-drug conjugate (ADC), a targeted therapy, or other anticancer drugs.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

### Embodiments

This invention provides the following non-limiting embodiments.

Embodiment 1 is a multispecific binding molecule comprising:

(i) a first binding domain that specifically binds to a lymphotoxin beta receptor (LTBR), and

(ii) a second binding domain that specifically binds to extra domain B of fibronectin (EDB), wherein the multispecific binding molecule activates LTBR upon binding of the EDB.

Embodiment 2 is the multispecific binding molecule of embodiment 1, wherein the multispecific binding molecule activates LTBR in a tumor specific manner.

Embodiment 3 is the multispecific binding molecule of embodiment 1 or 2, wherein the multispecific binding molecule is a bispecific antibody.

Embodiment 4 is the multispecific binding molecule of any one of embodiments 1 to 3, wherein the multispecific binding molecule comprises two antigen binding domains.

Embodiment 5 is the multispecific binding molecule of any one of embodiments 1 to 3, wherein the multispecific binding molecule comprises three antigen binding domains.

Embodiment 6 is the multispecific binding molecule of embodiment 5, wherein the three antigen binding domains comprise one binding domain that specifically binds to LTBR.

Embodiment 7 is the multispecific binding molecule of embodiment 5 or 6, wherein the three antigen binding domains comprise two binding domains that specifically bind EDB.

Embodiment 8 is the multispecific binding molecule of any one of embodiments 5 to 7, wherein the binding domain that specifically binds to LTBR comprises a single chain variable domain of an antibody.

Embodiment 9 is the multispecific binding molecule of any one of embodiments 1 to 8, wherein the first binding domain that specifically binds to LTBR comprises a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said VH and VL comprise any one or more of the following [(i) through (viii)]:

(i) HCDR1, HCDR2 and HCDR3 comprising the amino acid sequences of SEQ ID NO:60, SEQ ID NO:61, and SEQ ID NO:62, respectively, and LCDR1, LCDR2 and LCDR3 comprising the amino acid sequences of SEQ ID NO:63, SEQ ID NO:64, and SEQ ID NO:65, respectively; or
(ii) HCDR1, HCDR2 and HCDR3 comprising the amino acid sequences of SEQ ID NO:83, SEQ ID NO:61, and SEQ ID NO:62, respectively, and LCDR1, LCDR2 and LCDR3 comprising the amino acid sequences of SEQ ID NO:63, SEQ ID NO:64, and SEQ ID NO:65, respectively; or
(iii) HCDR1, HCDR2 and HCDR3 comprising the amino acid sequences of SEQ ID NO:66, SEQ ID NO:67, and SEQ ID NO:68, respectively, and LCDR1, LCDR2, and LCDR3 comprising the amino acid sequences of SEQ ID NO:69, SEQ ID NO:70, and SEQ ID NO:71, respectively; or
(iv) VH comprises an amino acid sequence having at least 95%, 96%, 97%, 98%, 99% identity or 100% identity to the amino acid sequence of SEQ ID NO:43, and VL comprises an amino acid sequence having at least 95%, 96%, 97%, 98%, 99% identity or 100% identity to the amino acid sequence of SEQ ID NO:44, such as wherein VH comprises an amino acid sequence having at least 95% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 96% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 97% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 98% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having at least 99% identity to the amino acid sequence of SEQ ID NO:43, and VL comprises an amino acid sequence having at least 95%, 96%, 97%, 98%, 99% identity or 100% identity to the amino acid sequence of SEQ ID NO:44; VH comprises an amino acid sequence having 100% identity to the amino acid sequence of SEQ ID NO:43, and VL comprises an amino acid sequence having at least 95%, 96%, 97%, 98%, 99% identity or 100% identity to the amino acid sequence of SEQ ID NO:44; or
(v) VH comprises an amino acid sequence having at least 95%, 96%, 97%, 98%, 99% identity or 100% identity to the amino acid sequence of SEQ ID NO:47, and VL comprises an amino acid sequence having at least 95%, 96%, 97%, 98%, 99% identity or 100% identity to the amino acid sequence of SEQ ID NO:48, such as wherein VH comprises an amino acid sequence having at least 95% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 96% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 97% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 98% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having at least 99% identity to the amino acid sequence of SEQ ID NO:47, and VL comprises an amino acid sequence having at least 95%, 96%, 97%, 98%, 99% identity or 100% identity to the amino acid sequence of SEQ ID NO:48; VH comprises an amino acid sequence having 100% identity to the amino acid sequence of SEQ ID NO:47, and VL comprises an amino acid sequence having at least 95%, 96%, 97%, 98%, 99% identity or 100% identity to the amino acid sequence of SEQ ID NO:48: or

### (vi) SEQ ID NO: 22; or

(vii) SEQ ID NO: 23; or
(viii) SEQ ID NO: 25.

Embodiment 10 is the multispecific binding molecule of any one of embodiments 1 to 9, wherein the second binding domain that specifically binds to EDB comprises a heavy chain variable region (VH) and a light chain variable region (VL), wherein the VH comprises a heavy chain complementarity determining region 1 (HCDR1), a HCDR2, and a HCDR3, and the VL comprises a light chain complementarity determining region 1 (LCDR1), a LCDR2, and a LCDR3, wherein said VH and VL comprise any one or more of the following [(i) through (ii)]:

(i) HCDR1, HCDR2 and HCDR3 comprising the amino acid sequences of SEQ ID NO:72, SEQ ID NO:73, and SEQ ID NO:74, respectively, and LCDR1, LCDR2 and LCDR3 comprising the amino acid sequences of SEQ ID NO:75, SEQ ID NO:76, and SEQ ID NO:77, respectively; or
(ii) VH comprises an amino acid sequence having at least 95%, 96%, 97%, 98%, 99% identity or 100% identity to the amino acid sequence of SEQ ID NO:45, and VL comprises an amino acid sequence having at least 95%, 96%, 97%, 98%, 99% identity or 100% identity to the amino acid sequence of SEQ ID NO:46, such as wherein VH comprises an amino acid sequence having at least 95% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 96% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 97% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 98% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having at least 99% identity to the amino acid sequence of SEQ ID NO:45, and VL comprises an amino acid sequence having at least 95%, 96%, 97%, 98%, 99% identity or 100% identity to the amino acid sequence of SEQ ID NO:46; VH comprises an amino acid sequence having 100% identity to the amino acid sequence of SEQ ID NO:45, and VL comprises an amino acid sequence having at least 95%, 96%, 97%, 98%, 99% identity or 100% identity to the amino acid sequence of SEQ ID NO:46.

Embodiment 11 is the multispecific binding molecule of any one of embodiments 1 to 10, comprising any one or more of [(a) through (1)]:

(a) (i) a first heavy chain comprising the amino acid sequence of SEQ ID NO: 1 forming a binding domain with a first light chain comprising the amino acid sequence of SEQ ID NO: 2, and
(ii) a second heavy chain comprising the amino acid sequence of SEQ ID NO: 4 forming a binding domain with a second light chain comprising the amino acid sequence of SEQ ID NO: 5; or
(b) (i) a first heavy chain comprising the amino acid sequence of SEQ ID NO: 9 forming a binding domain with a first light chain comprising the amino acid sequence of SEQ ID NO: 10, and (ii) a second heavy chain comprising the amino acid sequence of SEQ ID NO: 4 forming a binding domain with a second light chain comprising the amino acid sequence of SEQ ID NO: 5.

Embodiment 12 is the multispecific binding molecule of any one of embodiments 1 to 10, comprising any of:

(c) (i) an scFv-heavy chain fusion comprising the amino acid sequence of SEQ ID NO: 30, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5; or
(d) (i) an scFv-heavy chain fusion comprising the amino acid sequence of SEQ ID NO: 31, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5; or
(e) (i) an scFv-heavy chain fusion comprising the amino acid sequence of SEQ ID NO: 32, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5; or
(f) (i) an scFv-heavy chain fusion comprising the amino acid sequence of SEQ ID NO: 33, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5; or
(g) (i) an scFv-heavy chain fusion comprising the amino acid sequence of SEQ ID NO: 34, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5; or
(h) (i) an scFv-heavy chain fusion comprising the amino acid sequence of SEQ ID NO: 35, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5; or
(j) (i) an scFv-heavy chain fusion comprising the amino acid sequence of SEQ ID NO: 38, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5; or
(k) (i) an scFv-heavy chain fusion comprising the amino acid sequence of SEQ ID NO: 39, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5; or
(l) (i) an scFv-heavy chain fusion comprising the amino acid sequence of SEQ ID NO: 56, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5.

Embodiment 13 is the multispecific binding molecule of any one of embodiments 1 to 10, comprising (i) an scFv-heavy chain fusion comprising the amino acid sequence of SEQ ID NO: 38, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5 Embodiment 14 is one or more nucleic acid molecules encoding the multispecific binding molecule of any one of embodiments 1 to 13.

Embodiment 15 is one or more vectors comprising the one or more nucleic acid molecules of embodiment 14.

Embodiment 16 is an isolated host cell comprising the one or more vectors of embodiment 15.

Embodiment 17 is a pharmaceutical composition comprising the multispecific binding molecule of any one of embodiments 1 to 13 and a pharmaceutically acceptable carrier.

Embodiment 18 is a method of treating cancer in a subject in need thereof, comprising administering to the subject the multispecific binding molecule of any one of embodiments 1 to 14, the one or more nucleic acid molecules of embodiment 15, the one or more vectors of embodiment 16, or the pharmaceutical composition of embodiment 17.

Embodiment 19 is the use of the multispecific binding molecule of any one of embodiments 1 to 14, the one or more nucleic acid molecules of embodiment 15, the one or more vectors of embodiment 16, or the pharmaceutical composition of embodiment 17, for activating LTBR in tumor tissue.

Embodiment 20 is a method of producing a multispecific binding molecule of any one of embodiments 1 to 13, the method comprising expressing the one or more nucleic acid molecules of embodiment 14 or the one or more vectors of embodiment 15 in a host cell and harvesting the multispecific binding molecule.

Embodiment 21 is the multispecific molecule of any one of embodiments 1-10, which induces NF-κB signaling in the presence of EDB that is at least 2-fold, such as at least 3-fold, for example at least 4-fold greater than the NF-κB signaling induced in the absence of EDB.

Embodiment 22 is the multispecific molecule of any one of embodiments 1-10 or 21, which induces ICAM-1 expression of the surface of cells in the presence of EDB that is at least 2-fold, such as at least 3-fold, for example at least 4-fold greater than the ICAM-1 expression induced in the absence of EDB.

### EXAMPLES

### Example 1: Generation of EDB/LTBR Bispecific Antibodies and Control Molecules

Bispecific antibodies and control molecules, derived from the target binding sequences shown in Table 1, were transiently expressed in CHO suspension cultures in serum-free/animal component-free media, and purified by protein A affinity chromatography, followed by preparative size exclusion chromatography (SEC) on a Superdex 200 10/300 GL column (GE Healthcare) using a Äkta Pure instrument (GE Healthcare). Heavy chains contained knob-into-hole (KiH) mutations to promote heterodimerization (Ridgway et al., Protein Eng. 9(7):617-21 (1996); Atwell et al., J. Mol. Biol. 270(1):26-35 (1997); Merchant et al., Nat. Biotechnol. 16(7):677-81 (1998)). Antibodies contained the IgG1sigma Fc comprising a set of seven Fc mutations—L234A, L235A, G237A, P238S, H268A, A330S, and P331S—when compared to the wild type IgG1 to reduce Fc receptor interactions (Tam et al., Antibodies (2017)).

Symmetric mono- and bispecific antibodies were generated with IgG1sigma mutations, without KiH mutations.

TABLE 1
Target binding sequences used for the constructs of Example 1.

| Antibody/Ligand | | | |
|---|---|---|---|
| designation | Target/receptor | VH or Chain 1 | VL or Chain 2 |
| LTBRmAb1 | human LTBR | WO2004002431/SEQ ID NO. 43 | WO2004002431/SEQ ID |
| (BHA10) | | | NO. 44 |
| LTBRmAb2 | human LTBR | WO0230986/SEQ ID NO. 47 | WO0230986/SEQ ID |
| (CBE11) | | | NO. 48 |
| EDBmAb1* | human extra-domain B | WO9745544/SEQ ID NO: 45 | WO9745544/SEQ ID |
| (L19) | (EDB) of fibronectin | | NO: 46 |
| B21M | RSV | Vafa O et al. (2014) Methods | Vafa O et al. (2014) Methods |
| | | 65(1)/SEQ ID NO: 49 | 65(1)/SEQ ID NO: 50 |
| 3xhmLIGHT | human LTBR and | Tang et al 2016 Cancer Cell SEQ | — |
| | human HVEM | ID NO: 16 | |
| LTα1β2 | human LTBR | WO2018119118/SEQ ID NO: 17 | — |
| MSLNmAb1 | human Mesothelin | EP2322560/SEQ ID NO: 78 | EP2322560/SEQ ID NO: 79 |

*EDBmAb1(WO9745544) used here is an anti-ED-B antibody that has been tested in the clinic, other antibodies binding to ED-B or to adjacent domains have been described previously (Carnemolla et al. Int. J. Cancer: 68, 397-405 (1996))

Protein concentration was determined by absorbance measurement at 280 nm (OD280) and purification yield determined. Analytical SEC was performed using a Bio SEC-5 column (Agilent, 5 μm particle size, 300 Å) on a Thermo Vanquish HPLC system. 10 μl purified protein was loaded on the column and elution was recorded by OD280.

Table 2 shows an overview of structural properties of the bispecific antibodies and control molecules described in this example. The molecules in boldface are molecules according to the invention, while the others are controls for different aspects.

Table 3 shows structural properties of another comparative bispecific antibody, targeting LTBR and mesothelin (a tumor associated antigen not present in the extracellular matrix), as discussed in comparative example 4.

TABLE 2
Overview of the structural properties of the EDB/LTBR bispecific antibodies and control molecules - Part 1

| | | | Binding sites | | | | | |
|---|---|---|---|---|---|---|---|---|
| | | | | | | | | B21M |
| | | | | | | | | (isotype |
| | | | | | | | | control |
| | Name | Format | LTBRmAb1 | LTBRmAb2 | EDBmAb1 | LIGHT | LTα1β2 | mAb) |
| Asymmetric | COVA14121 | 1:1 | 1 | — | 1 | — | — | — |
| antibodies; | COVA14122 | 1:1 | — | 1 | 1 | — | — | — |
| 1:1 format | COVA14120 | 1:1 | 1 | — | — | — | — | 1 |
| | COVA14123 | 1:1 | — | 1 | — | — | — | 1 |
| | COVA14124 | 1:1 | — | — | 1 | — | — | 1 |
| Asymmetric | COVA1480 | 2:1 | 1 (scFv) | — | 2 (mAb) | — | — | — |
| antibodies; | COVA1481 | 2:1 | 1 (scFv) | — | 2 (mAb) | — | — | — |
| 2:1 format | COVA1482 | 2:1 | 1 (scFv) | — | 2 (mAb) | — | — | — |
| | COVA1483 | 2:1 | 1 (scFv) | — | 2 (mAb) | — | — | — |
| | COVA1484 | 2:1 | 1 (scFv) | — | — | — | — | 2 (mAb) |
| | COVA1485 | 2:1 | 1 (scFv) | — | — | — | — | 2 (mAb) |
| | COVA1486 | 2:1 | 1 (scFv) | — | — | — | — | 2 (mAb) |
| | COVA1487 | 2:1 | 1 (scFv) | — | — | — | — | 2 (mAb) |

| | | Fc-domain | scFv features | | | |
|---|---|---|---|---|---|---|
| | | Protein A | Stapled | Disulfide | | |
| | Name | mut.* | linker | stabilized | orientation | Comments |
| Asymmetric | COVA14121 | no | — | — | | |
| antibodies; | COVA14122 | no | — | — | | |
| 1:1 format | COVA14120 | no | — | — | | LTBR/null |
| | | | | | | control for |
| | | | | | | COVA14121 |
| | COVA14123 | no | — | — | | LTBR/null |
| | | | | | | control for |
| | | | | | | COVA14122 |
| | COVA14124 | no | — | — | | EDB/null |
| | | | | | | control for |
| | | | | | | COVA14121 |
| | | | | | | and |
| | | | | | | COVA14122 |
| Asymmetric | COVA1480 | yes | yes | — | VH-VL | scFv fused to N- |
| antibodies; | | | | | | term of |
| 2:1 format | | | | | | EDBmAb1 HC |
| | COVA1481 | yes | yes | — | VL-VH | scFv fused to N- |
| | | | | | | term of |
| | | | | | | EDBmAb1 HC |
| | COVA1482 | yes | yes | — | VH-VL | scFv fused to C- |
| | | | | | | term of |
| | | | | | | EDBmAb1 HC |
| | COVA1483 | yes | yes | — | VL-VH | scFv fused to C- |
| | | | | | | term of |
| | | | | | | EDBmAb1 HC |
| | COVA1484 | yes | yes | — | VH-VL | LTBR/null |
| | | | | | | control to |
| | | | | | | COVA1480 |
| | COVA1485 | yes | yes | — | VL-VH | LTBR/null |
| | | | | | | control to |
| | | | | | | COVA1481 |
| | COVA1486 | yes | yes | — | VH-VL | LTBR/null |
| | | | | | | control to |
| | | | | | | COVA1482 and |
| | | | | | | to COVA14146 |
| | COVA1487 | yes | yes | — | VL-VH | LTBR/null |
| | | | | | | control to |
| | | | | | | COVA1483 |

TABLE 2
Overview of the structural properties of the EDB/LTBR
bispecific antibodies and control molecules - Part 2

| | | | Binding sites | | | | | |
|---|---|---|---|---|---|---|---|---|
| | | | | | | | | B21M |
| | | | | | | | | (isotype |
| | | | | | | | | control |
| | Name | Format | LTBRmAb1 | LTBRmAb2 | EDBmAb1 | LIGHT | LTα1β2 | mAb) |
| Asymmetric | COVA14107 | 2:1 | 1 (scFv) | — | 2 (mAb) | — | — | — |
| antibodies; | COVA14108 | 2:1 | 1 (scFv) | — | 2 (mAb) | — | — | — |
| 2:1 format | COVA14133 | 2:1 | 1 (scFv) | — | 2 (mAb) | — | — | — |
| | COVA14136 | 2:1 | 1 (scFv) | — | — | — | — | 2(mAb) |
| | COVA14174 | 2:1 | 1 (scFv) | — | 2 (mAb) | — | — | — |
| | COVA14175 | 2:1 | 1 (scFv) | — | — | — | — | 2(mAb) |
| | COVA1456 | 2:1 | 1 (scFv) | — | 2 (mAb) | — | — | — |
| | COVA1462 | 2:1 | 1 (scFv) | — | — | — | — | 2(mAb) |

| | | Fc-domain | scFv features | | | |
|---|---|---|---|---|---|---|
| | | Protein A | Stapled | Disulfide | | |
| | Name | mut.* | linker | stabilized | orientation | Comments |
| Asymmetric | COVA14107 | yes | yes | no | VH-VL | scFv (C-Term |
| antibodies; | | | | | | fusion to |
| 2:1 format | | | | | | EDBmAb1 HC) |
| | | | | | | contains mutations |
| | | | | | | for reduced affinity |
| | | | | | | to LTBR (VL3: |
| | | | | | | Y36F_S49Y_F87) |
| | COVA14108 | yes | yes | no | VH-VL | scFv (C-Term |
| | | | | | | fusion to |
| | | | | | | EDBmAb1 HC) |
| | | | | | | ontains mutations |
| | | | | | | for reduced affinity |
| | | | | | | to LTBR |
| | | | | | | (VH_CDR1_Y33A) |
| | COVA14133 | no | yes | no | VH-VL | scFv fused to C- |
| | | | | | | term of EDBmAb1 |
| | | | | | | HC |
| | COVA14136 | no | yes | no | VH-VL | LTBR/null control |
| | | | | | | to COVA14133 |
| | COVA14174 | no | no | yes | VH-VL | Same as |
| | | | | | | COVA14133 but |
| | | | | | | with different |
| | | | | | | stabilization of |
| | | | | | | scFv |
| | COVA14175 | no | no | yes | VH-VL | LTBR/null control |
| | | | | | | to COVA14174 |
| | COVA1456 | yes | no | yes | VH-VL | Same as |
| | | | | | | COVA1482 but |
| | | | | | | with different |
| | | | | | | stabilization of |
| | | | | | | scFv |
| | COVA1462 | yes | no | yes | VH-VL | LTBR/null control |
| | | | | | | to COVA1456 |

TABLE 2
Overview of the structural properties of the EDB/LTBR
bispecific antibodies and control molecules - Part 3

| | | | Binding sites | | | | | |
|---|---|---|---|---|---|---|---|---|
| | | | | | | | | B21M |
| | | | | | | | | (isotype |
| | | | | | | | | control |
| | Name | Format | LTBRmAb1 | LTBRmAb2 | EDBmAb1 | LIGHT | LTα1β2 | mAb) |
| Antibody | COVA1454 | 1:1 | — | — | 1 | 1 | — | — |
| | COVA1418 | 1:1 | — | — | — | 1 | — | 1 |
| Antibody | COVA14113 | 2:2 | — | — | 2(mAb) | — | 2 | — |
| LTα1β2 | COVA14114 | 2:2 | — | — | — | — | 2 | 2(mAb) |
| fusions | COVA14116 | 2:1 | — | — | 2(mAb) | — | 1 | — |
| | COVA14117 | 2:1 | — | — | — | — | 1 | 2(mAb) |
| Antibodies | COVA1413 | 2:0 | 2(mAb) | — | — | — | — | — |
| | COVA1402 | 2:0 | — | 2(mAb) | — | — | — | — |
| | COVA1440 | 2:0 | — | — | — | — | — | 2(mAb) |
| | COVA1452 | 2:0 | — | — | 2(mAb) | — | — | — |

| | | Fc-domain | scFv features | | | |
|---|---|---|---|---|---|---|
| | | Protein A | Stapled | Disulfide | | |
| | Name | mut.* | linker | stabilized | orientation | Comments |
| Antibody | COVA1454 | yes | — | — | — | |
| | COVA1418 | yes | — | — | — | LIGHT/null |
| | | | | | | control for |
| | | | | | | COVA1454 |
| Antibody | COVA14113 | no | — | — | — | |
| LTα1β2 | COVA14114 | no | — | — | — | LTα1β2/null |
| fusions | | | | | | control to |
| | | | | | | COVA14113 |
| | COVA14116 | yes | — | — | — | |
| | COVA14117 | yes | — | — | — | LTα1β2/null |
| | | | | | | control to |
| | | | | | | COVA14116 |
| Antibodies | COVA1413 | no | — | — | — | |
| | COVA1402 | no | — | — | — | |
| | COVA1440 | no | — | — | — | |
| | COVA1452 | no | — | — | — | |

*mutations in the Fc portion to abrogate binding to protein A and facilitate purification of heterodimers, described in WO2010151792.

TABLE 3
Overview of the structural properties of the MSLN/LTBR bispecific antibody

| | | | | Fc-domain | scFv features | | | |
|---|---|---|---|---|---|---|---|---|
| | | Binding sites | | Protein A | Stapled | Disulfide | | |
| Name | Format | LTBRmAb1 | MSLNmAb1 | mut. | linker | stabilized | orientation | Comments |
| COVA14146 | 2:1 | 1 (scFv) | 2(mAb) | yes | yes | no | VH-VL | HC C-terminal fusion of |
| | | | | | | | | scFv derived from |
| | | | | | | | | LTBRmAb1 to |
| | | | | | | | | MSLNmAb1. Isotype |
| | | | | | | | | control molecule for this |
| | | | | | | | | construct is COVA1486 |

Described below is how the different constructs were generated.

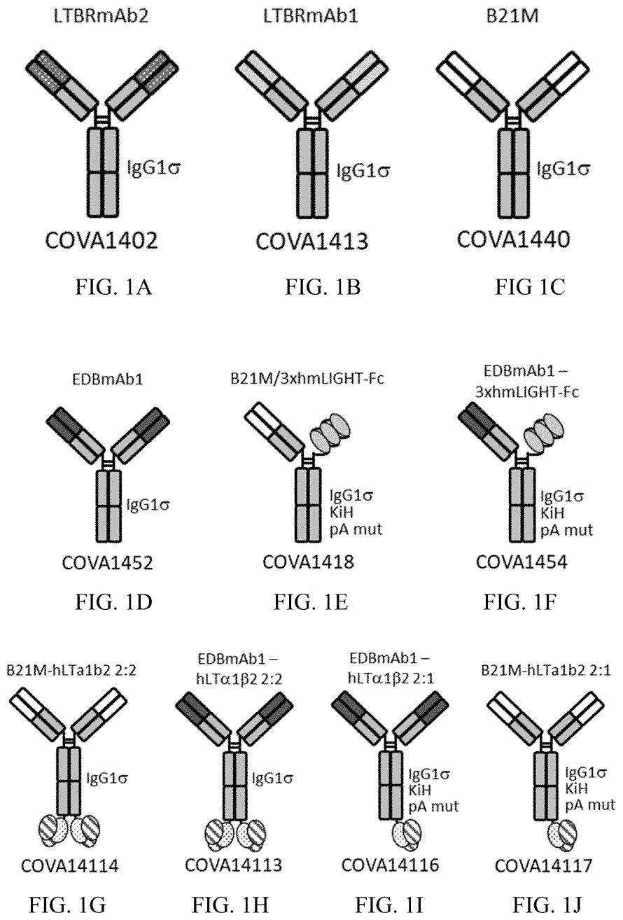

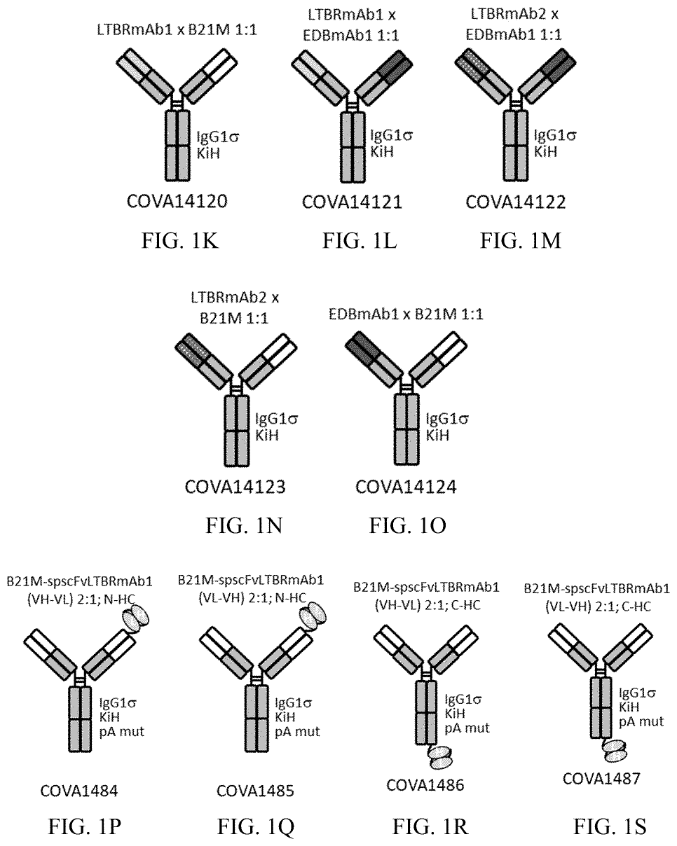

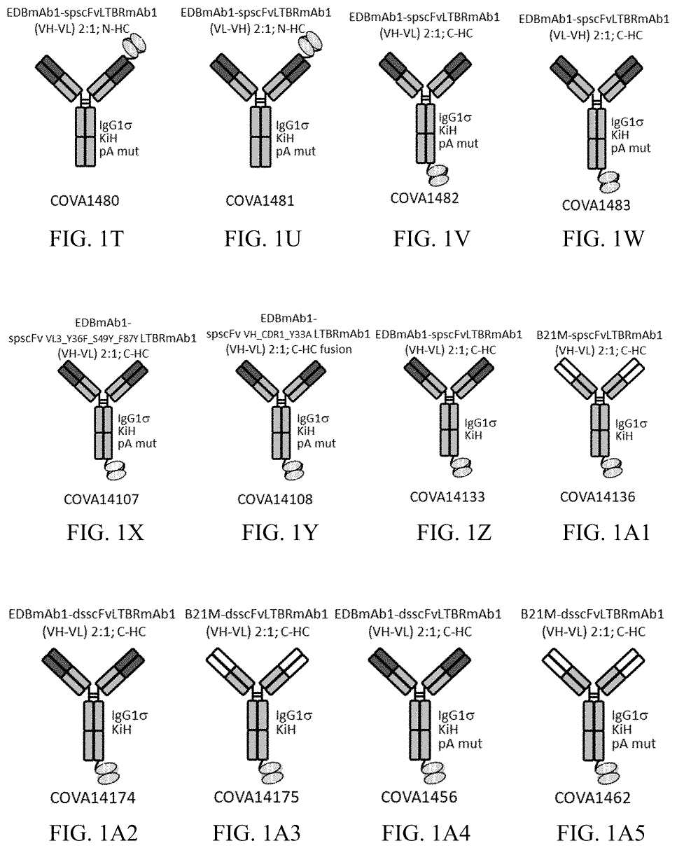

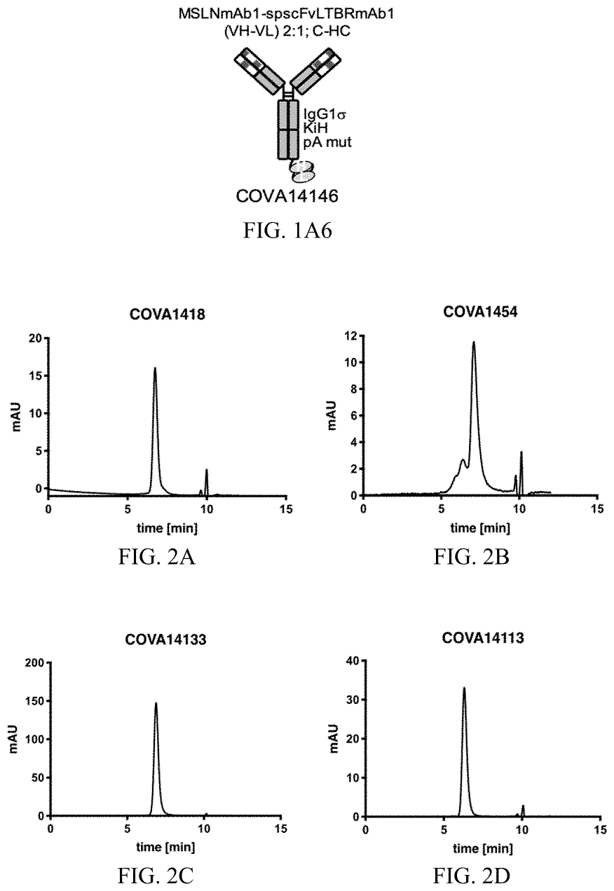

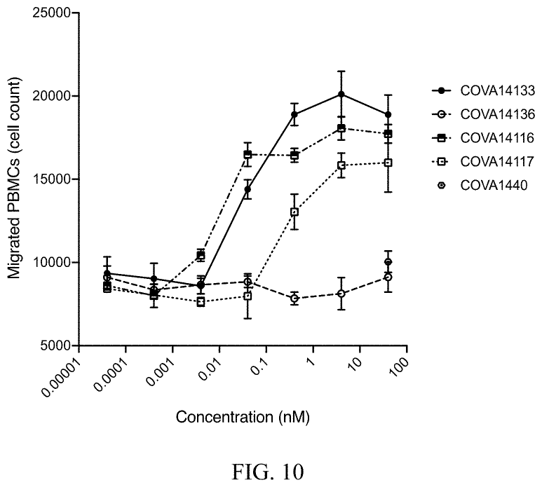

Asymmetric Antibodies, with 1:1 Stoichiometry (all IgG1sigma; all with Knob-into-Hole (KiH) Mutations):
i. COVA14121 was generated by co-expression of the heavy chain (HC; SEQ ID NO: 1) and the light chain (LC; SEQ ID NO: 2) of an agonistic LTBR antibody LTBRmAb1 and the heavy chain (HC; SEQ ID NO: 4) and light chain (LC; SEQ ID NO. 5) of an anti-EDB antibody EDBmAb1 (FIG. 1L).
ii. COVA14120 was generated by co-expression of the heavy chain (HC; SEQ ID NO: 1) and the light chain (LC; SEQ ID NO: 2) of an agonistic LTBR antibody LTBRmAb1 and the heavy chain (HC; SEQ ID NO: 7) and light chain (LC; SEQ ID NO. 8) of an anti-RSV antibody B21M (FIG. 1K).
iii. COVA14122 was generated by co-expression of the heavy chain (HC; SEQ ID NO: 9) and the light chain (LC; SEQ ID NO: 10) of an agonistic LTBR antibody LTBRmAb2 and the heavy chain (HC; SEQ ID NO: 4) and light chain (LC; SEQ ID NO. 5) of an anti-EDB antibody EDBmAb1 (FIG. 1M).
iv. COVA14123 was generated by co-expression of the heavy chain (HC; SEQ ID NO: 9) and the light chain (LC; SEQ ID NO: 10) of an agonistic LTBR antibody LTBRmAb2 and the heavy chain (HC; SEQ ID NO: 7) and light chain (LC; SEQ ID NO. 8) of an anti-RSV antibody B21M (FIG. 1N).
v. COVA14124 was generated by co-expression of the heavy chain (HC; SEQ ID NO: 4) and light chain (LC; SEQ ID NO. 5) of an anti-EDB antibody EDBmAb1 and the heavy chain (HC; SEQ ID NO: 6) and light chain (LC; SEQ ID NO. 8) of an anti-RSV antibody B21M (FIG. 10).
vi. COVA1454 was generated by co-expression of 3×hmLIGHT-Fc (SEQ ID NO: 15) with the heavy chain (HC; SEQ ID NO: 4) and light chain (LC; SEQ ID NO. 5) of an anti-EDB antibody EDBmAb1 (FIG. 1F). 3×hmLIGHT-Fc is a single-chain trimeric LIGHT engineered for better stability and for human and mouse cross-reactivity (Tang et al., Cancer Cell 29:285-96 (2016)) fused to the N-terminus of the IgG1sigma Fc.
vii. COVA1418 was generated by co-expression of 3×hmLIGHT-Fc (SEQ ID NO: 15) with the heavy chain (HC; SEQ ID NO:7) and light chain (LC; SEQ ID NO. 8) of an anti-RSV antibody B21M (FIG. 1E). 3×hmLIGHT-Fc is a single-chain trimeric LIGHT engineered for better stability and for human and mouse cross-reactivity (Tang et al., Cancer Cell 29:285-96 (2016)) fused to the N-terminus of the IgG1sigma Fc (SEQ ID NO: 58).

### Symmetric Antibodies (all IgG1sigma, No KiH Mutations):

viii. COVA14114 was generated by expression of the anti-RSV B21M antibody heavy chain carrying a C-terminal LTα1β2 fusion (SEQ ID NO: 18) with the light chain (LC; SEQ ID NO:8) of an anti-RSV B21M antibody (FIG. 1G).
ix. COVA14113 was generated by expression of the EDBmAb1 heavy chain carrying a C-terminal LTα1β2 fusion (SEQ ID NO: 20) with the light chain (LC; SEQ ID NO. 5) of an anti-EDB antibody EDBmAb1 (FIG. 1H).
x. COVA1413 was generated by co-expression of the heavy chain (HC; SEQ ID NO: 11) and the light chain (LC; SEQ ID NO: 2) of an agonistic LTBR antibody LTBRmAb1 (FIG. 1B).
xi. COVA1402 was generated by co-expression of the heavy chain (HC; SEQ ID NO: 13) and the light chain (LC; SEQ ID NO: 10) of an agonistic LTBR antibody LTBRmAb2 (FIG. 1A).
xii. COVA1440 was generated by co-expression of the heavy chain (HC; SEQ ID NO: 14) and light chain (LC; SEQ ID NO. 8) of an anti-RSV antibody B21M (FIG. 1C).
xiii. COVA1452 was generated by co-expression of the heavy chain (HC; SEQ ID NO: 12) and light chain (LC; SEQ ID NO. 5) of an anti-EDB antibody EDBmAb1 (FIG. 1D).
   Asymmetric Antibodies, with 2:1 Stoichiometry (all IgG1sigma, all with KiH Mutations)
xiv. COVA14116 was generated by co-expression of the EDBmAb1 heavy chain carrying a C-terminal LTα1β2 fusion (SEQ ID NO: 21, comprising SEQ ID NO: 84) with the heavy chain (HC; SEQ ID NO: 4) and light chain (LC; SEQ ID NO. 5) of an anti-EDB antibody EDBmAb1 (FIG. 1I).
xv. COVA14117 was generated by co-expression of the anti-RSV B21M antibody heavy chain carrying a C-terminal LTα1β2 fusion (SEQ ID NO: 19, comprising SEQ ID NO: 85) with the heavy chain (HC; SEQ ID NO: 7) and light chain (LC; SEQ ID NO: 8) of the anti-RSV B21M antibody (FIG. 1J).
xvi. COVA1484 was generated by co-expression of the anti-RSV B21M antibody heavy chain carrying a N-terminal stapled scFv BHA10 (VH-VL orientation SEQ ID NO: 22) fusion (SEQ ID NO: 26, comprising SEQ ID NO: 85) with the heavy chain (HC; SEQ ID NO: 7) and light chain (LC; SEQ ID NO: 8) of the anti-RSV B21M antibody (FIG. 1P).
xvii. COVA1485 was generated by co-expression of the anti-RSV B21M antibody heavy chain carrying a N-terminal stapled scFv BHA10 (VL-VH orientation SEQ ID NO: 23) fusion (SEQ ID NO: 27, comprising SEQ ID NO: 85) with the heavy chain (HC; SEQ ID NO: 7) and light chain (LC; SEQ ID NO: 8) of the anti-RSV B21M antibody (FIG. 1Q).
xviii. COVA1486 was generated by co-expression of the anti-RSV B21M antibody heavy chain carrying a C-terminal stapled scFv BHA10 (VH-VL orientation SEQ ID NO: 22) fusion (SEQ ID NO: 28, comprising SEQ ID NO: 85) with the heavy chain (HC; SEQ ID NO: 7) and light chain (LC; SEQ ID NO: 8) of the anti-RSV B21M antibody (FIG. 1R).
xix. COVA1487 was generated by co-expression of the anti-RSV B21M antibody heavy chain carrying a C-terminal stapled scFv BHA10 (VL-VH orientation SEQ ID NO: 23) fusion (SEQ ID NO: 29, comprising SEQ ID NO: 85) with the heavy chain (HC; SEQ ID NO: 7) and light chain (LC; SEQ ID NO: 8) of the anti-RSV B21M antibody (FIG. 1S).
xx. COVA1480 was generated by co-expression of an anti-EDB antibody EDBmAb1 heavy chain carrying a N-terminal stapled scFv BHA10 (VH-VL orientation SEQ ID NO: 22) fusion (SEQ ID NO: 30, comprising SEQ ID NO: 84) with the heavy chain (HC; SEQ ID NO: 4) and light chain (LC; SEQ ID NO: 5) of an anti-EDB antibody EDBmAb1 (FIG. 1T).
xxi. COVA1481 was generated by co-expression of an anti-EDB antibody EDBmAb1 heavy chain carrying a N-terminal stapled scFv BHA10 (VL-VH orientation SEQ ID NO: 23) fusion (SEQ ID NO: 31, comprising SEQ ID NO: 84) with the heavy chain (HC; SEQ ID NO: 4) and light chain (LC; SEQ ID NO: 5) of an anti-EDB antibody EDBmAb1 (FIG. 1U).
xxii. COVA1482 was generated by co-expression of an anti-EDB antibody EDBmAb1 heavy chain carrying a C-terminal stapled scFv BHA10 (VH-VL orientation SEQ ID NO: 22) fusion (SEQ ID NO: 32, comprising SEQ ID NO: 84) with the heavy chain (HC; SEQ ID NO: 4) and light chain (LC; SEQ ID NO: 5) of an anti-EDB antibody EDBmAb1 (FIG. 1V).
xxiii. COVA1483 was generated by co-expression of an anti-EDB antibody EDBmAb1 heavy chain carrying a C-terminal stapled scFv BHA10 (VL-VH orientation SEQ ID NO: 23) fusion (SEQ ID NO: 33, comprising SEQ ID NO: 84) with the heavy chain (HC; SEQ ID NO: 4) and light chain (LC; SEQ ID NO: 5) of an anti-EDB antibody EDBmAb1 (FIG. 1W).
xxiv. COVA14107 was generated by co-expression of an anti-EDB antibody EDBmAb1 heavy chain carrying a C-terminal stapled scFv BHA10 (VH-VL orientation, VL3 Y36F_S49Y_F87Y SEQ ID NO: 53) fusion (SEQ ID NO: 34, comprising SEQ ID NO: 84) with the heavy chain (HC; SEQ ID NO: 4) and light chain (LC; SEQ ID NO: 5) of an anti-EDB antibody EDBmAb1 (FIG. 1X).
xxv. COVA14108 was generated by co-expression of an anti-EDB antibody EDBmAb1 heavy chain carrying a C-terminal stapled scFv BHA10 (VH-VL orientation, VH_CDR1_Y33A SEQ ID NO: 54) fusion (SEQ ID NO: 35, comprising SEQ ID NO: 84) with the heavy chain (HC; SEQ ID NO: 4) and light chain (LC; SEQ ID NO: 5) of an anti-EDB antibody EDBmAb1 (FIG. 1Y).
xxvi. COVA14133 was generated by co-expression of an anti-EDB antibody EDBmAb1 heavy chain carrying a C-terminal stapled scFv BHA10 (VH-VL orientation SEQ ID NO: 22) fusion (SEQ ID NO: 38, comprising SEQ ID NO: 3) with the heavy chain (HC; SEQ ID NO: 4) and light chain (LC; SEQ ID NO: 5) of an anti-EDB antibody EDBmAb1 (FIG. 1Z).
xxvii. COVA14136 was generated by co-expression of the anti-RSV B21M antibody heavy chain carrying a C-terminal stapled scFv BHA10 (VH-VL orientation SEQ ID NO: 22) fusion (SEQ ID NO: 41, comprising SEQ ID NO: 6) with the heavy chain (HC; SEQ ID NO: 7) and light chain (LC; SEQ ID NO: 8) of an anti-EDB antibody EDBmAb1 (FIG. 1A1).
xxviii. COVA14174 was generated by co-expression of an anti-EDB antibody EDBmAb1 heavy chain carrying a C-terminal disulfide stabilized scFv BHA10 (VH-VL orientation SEQ ID NO: 25) fusion (SEQ ID NO: 39, comprising SEQ ID NO: 3) with the heavy chain (HC; SEQ ID NO: 4) and light chain (LC; SEQ ID NO: 5) of an anti-EDB antibody EDBmAb1 (FIG. 1A2).
xxix. COVA14175 was generated by co-expression of the anti-RSV B21M antibody heavy chain carrying a C-terminal disulfide stabilized scFv BHA10 (VH-VL orientation SEQ ID NO: 25) fusion (SEQ ID NO: 40, comprising SEQ ID NO: 6) with the heavy chain (HC; SEQ ID NO: 7) and light chain (LC; SEQ ID NO: 8) of the anti-RSV B21M antibody (FIG. 1A3).
xxx. COVA1456 was generated by co-expression of an anti-EDB antibody EDBmAb1 heavy chain carrying a C-terminal disulfide stabilized scFv BHA10 (VH-VL orientation SEQ ID NO: 25) fusion (SEQ ID NO: 56, comprising SEQ ID NO: 84) with the heavy chain (HC; SEQ ID NO: 4) and light chain (LC; SEQ ID NO: 5) of an anti-EDB antibody EDBmAb1 (FIG. 1A4).
xxxi. COVA1462 was generated by co-expression of the anti-RSV B21M antibody heavy chain carrying a C-terminal disulfide stabilized scFv BHA10 (VH-VL orientation SEQ ID NO: 25) fusion (SEQ ID NO: 57, comprising SEQ ID NO: 85) with the heavy chain (HC; SEQ ID NO: 7) and light chain (LC; SEQ ID NO: 8) of the anti-RSV B21M antibody (FIG. 1A5).
   Mesothelin/LTBR Bispecific: Asymmetric Antibody, with 2:1 Stoichiometry (IgG1sigma, with KiH Mutations
xxxii. COVA14146 was generated by co-expression of an anti-Mesothelin antibody MSLNmAb1 heavy chain carrying a C-terminal stapled scFv BHA10 (VH-VL orientation SEQ ID NO:22) fusion (SEQ ID NO:80, comprising SEQ ID NO: 86) with the heavy chain (HC; SEQ ID NO:81) and light chain (LC; SEQ ID NO:82) of an anti-Mesothelin antibody MSLNmAb1 (FIG. 1A6 and Table 3).

Results

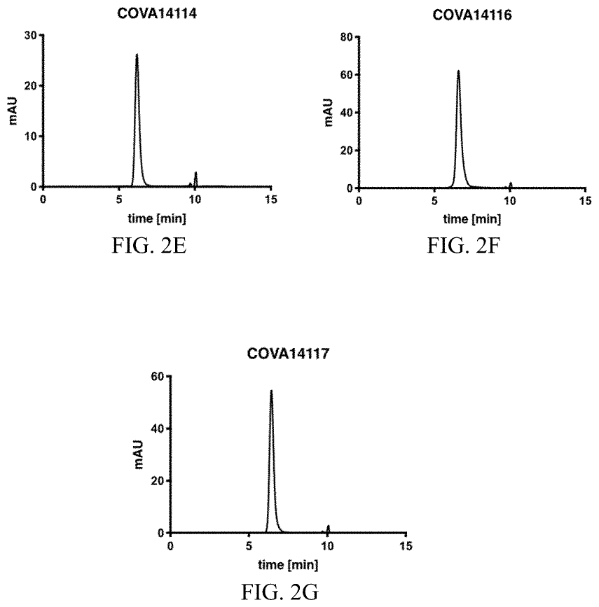

All constructs described above could be expressed and purified, however, surprisingly, the LIGHT and LTα1β2 containing constructs (COVA1418, COVA1454, COVA14113, COVA14114, COVA14116 and COVA14117; Table 2) showed up to 10-fold reduced purification yields compared to EDB/LTBR bispecifics containing a stapled scFv derived from an agonistic anti-LTBR antibody (e.g. COVA1482 and COVA14133; see Table 4). Moreover, the constructs containing LIGHT (e.g. COVA1454) showed a tendency to have a reduced monomeric content as can be seen from the size exclusion chromatogram shown in FIG. 2 and Table 4. Taken together, these facts (purification yields up to 10-fold higher and higher monomeric content) indicate that the bispecific constructs of this invention might have better biophysical properties than constructs comprising LIGHT or LTα1β2-Fc fusions.

TABLE 4
Yield and purities of selected EBD/LTBR bispecifics

| Bispecific | | Yield | Purity |
|---|---|---|---|
| name | Description | [mg/L] | [% monomer] |
| COVA1418 | 1:1 heterodimer consisting of B21M HC and LC | 6.6 | 100 |
| | paired with 3xhmLIGHT-Fc | | |
| COVA1454 | 1:1 heterodimer consisting of EDBmAb1 HC | 6.8 | 76.2 |
| | and LC paired with 3xhmLIGHT-Fc | | |
| COVA14113 | 2:2 homodimer consisting of EDBmAb1 HC | 11.0 | 99.8 |
| | fused to LTα1β2 paired with EDBmAb1 LC | | |
| COVA14114 | 2:2 homodimer consisting of B21M HC fused to | 8.0 | 100 |
| | LTα1β2 paired with B21M LC | | |
| COVA14116 | 2:1 heterodimer consisting of EDBmAb1 HC | 18.0 | 100 |
| | fused to LTα1β2 paired with EDBmAb1 HC | | |
| | and EDBmAb1 LC | | |
| COVA14117 | 2:1 heterodimer consisting of B21M HC fused to | 14.3 | 100 |
| | LTα1β2 paired with B21M HC and B21M LC | | |
| COVA1482 | 2:1 heterodimer consisting of EDBmAb1 HC | 53 | 100 |
| | fused to stapled scFv BHA10 (VH-VL) | | |
| | (contains mutation in the Fc that abrogates | | |
| | binding to Protein A to facilitate purification of | | |
| | heterodimer) paired with EDBmAb1 HC and | | |
| | EDBmAb1 LC | | |
| COVA14133 | 2:1 heterodimer consisting of EDBmAb1 HC | 64.3 | 100 |
| | fused to stapled scFv BHA10 (VH-VL) paired | | |
| | with EDBmAb1 HC and EDBmAb1 LC | | |

### Example 2: EDB Dependent In Vitro LTBR Activation—NF-κB Luciferase Reporter Assay

To show that the EDB/LTBR bispecifics are able to activate LTBR in an EDB-dependent way, the activity of the compounds was tested in an A549 cell NF-κB luciferase reporter assay in the presence or absence of EDB containing Fibronectin (EDB+Fibronectin). NF-κB signaling plays a pivotal role in regulating cell development and immune homeostasis. Activation of NF-κB through tumor necrosis factor receptors (TNFR) or the TNFR superfamily members (e.g., LTBR) occurs upon engagement with their respective ligands. The A549 lung epithelial cell line naturally expresses LTBR and the NF-κB luciferase reporter construct is stably integrated into the genome of the A549 lung epithelial cell line. Following activation by stimulants, endogenous NF-κB transcription factors bind to the DNA response elements to induce transcription of the luciferase gene.

To demonstrate EDB-dependent activation of LTBR, high binding 96-well μClear flat bottom plates (Greiner; Monroe, N.C.) were coated overnight with 150 ng/well human recombinant EDB+Fibronectin domains 7-B-8-9 (EDB+; SEQ ID NO: 51) or 150 ng/well human recombinant Fibronectin domains 7-8-9 (EDB−; SEQ ID NO: 52).

After overnight incubation, the coated plates were washed with PBS and blocked for 2 hours at 37° C. with assay medium (DMEM+10% heat inactivated FBS). A 1:5 dilution series of the compounds to be tested was prepared in assay medium as 2-fold concentration stocks (final concentrations tested ranged from 200 nM to 2.6 pM). 50 μl of diluted compounds were added to the pre-blocked plate after the blocking solution was removed by aspiration. 50 μl of a A549 cell suspension (concentration of cell suspension=0.4 Mio cells/ml assay medium) were added to each well (20,000 cells/well). A549 cells were previously detached from cell culture flask by using Accutase/EDTA and were then transferred in assay medium. Cells were incubated with the compounds for 18-20 hours at 37° C./5% CO₂.

After incubation for 18 hours, the Bio-Glo™ Luciferase Assay System (Promega; Madison, Wis.) was used to detect luciferase activity. Luminescence was measured using a Tecan M1000 Pro instrument with an integration time of 500 milliseconds. From the resulting relative light units (RLU), the fold induction of LTBR signaling was calculated as follows: Fold induction=RLU_{stimulated} cells/average RLU_{unstimulated} cells (unstimulated cells were included as control in each plate tested).

Dose response curves, including standard deviations, were plotted using GraphPad Prism, and non-linear fits were applied (log(agonist) vs. response (variable slope−three parameters)), if applicable. In order to fit the data, the x-values (concentrations of compounds) were transformed using the X=Log(x) function of GraphPad Prism.

Results

### Antibody-LIGHT Fusions

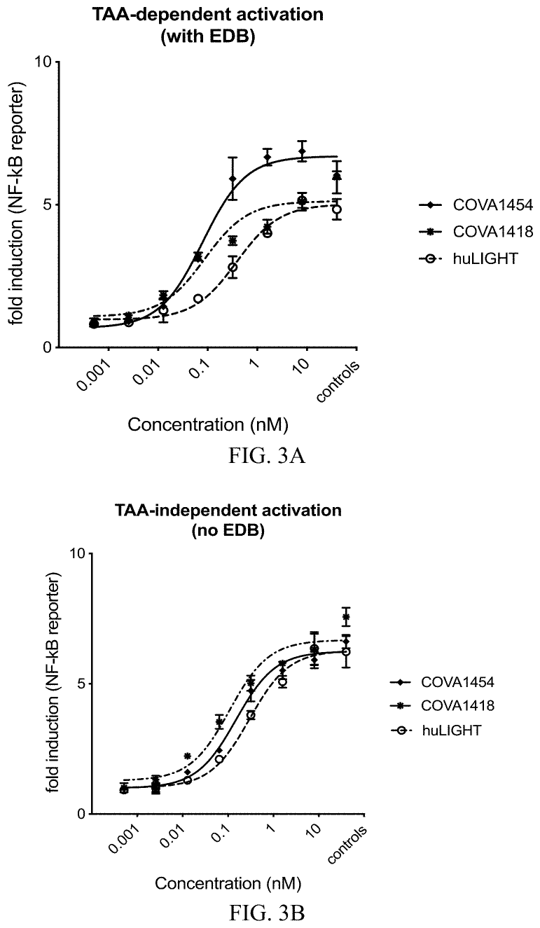

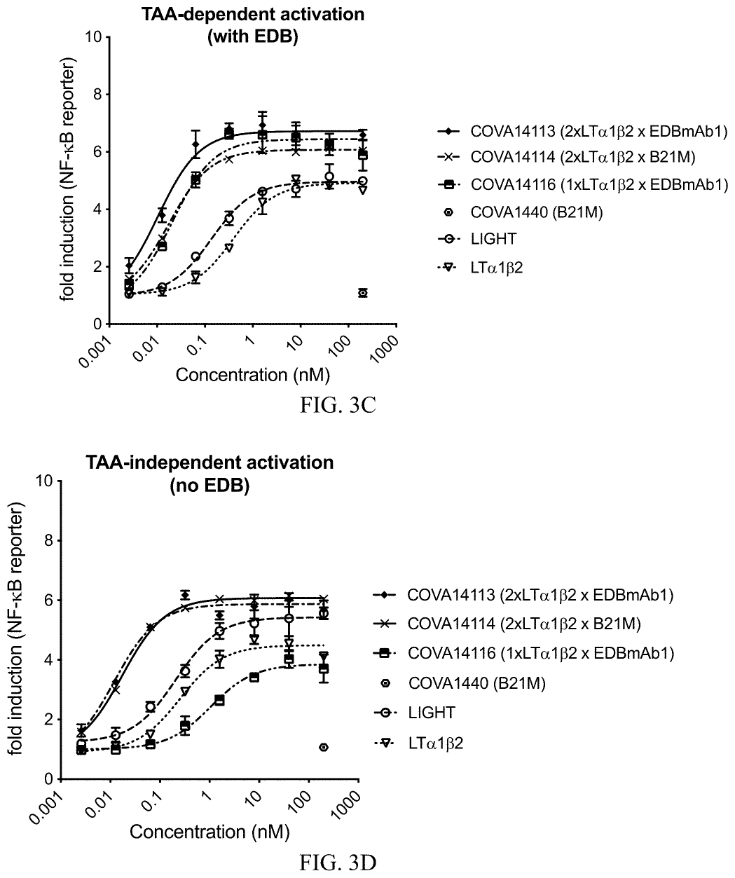

In analogy to the work published by Tang et al (Tang et al., Cancer Cell 29:285-96 (2016), where a bispecific molecule anti-EGFR-LIGHT fusion was described to have anti-tumor activity, COVA1454, a bispecific molecule consisting of one EDB binding arm and one LIGHT trimer-Fc fusion (FIG. 1F and Table 2), was designed, expressed, and tested in the A549 cell NF-κB luciferase reporter assay in the presence or absence of EDB containing Fibronectin. The activity of COVA1454 was compared to soluble recombinant human LIGHT (Cat. no. 664-LI-025/CF; R&D Systems; Minneapolis, Minn.) and non-targeted LIGHT (COVA1418; FIG. 1E and Table 2). FIG. 3A shows that, in presence of EDB containing Fibronectin, COVA1454 only slightly activates LTBR more than untargeted LIGHT (COVA1418) or soluble recombinant human LIGHT. Interestingly, FIG. 3B shows that, in absence of EDB containing Fibronectin, COVA1454, COVA1418 and soluble recombinant LIGHT activate LTBR to the same extent and to a similar degree as in presence of EDB (FIG. 3A). These findings, taken together with the broad expression of LTBR in normal tissue (Lukashev, et al. Cancer Res., 66(19):9617-24 (2006)), indicate, that antibody LIGHT fusions are not suitable to achieve tumor specific activation of LTBR. In fact, activation of LTBR in normal tissues could possibly lead to undesired off-tumor toxicities.

### Antibody-LTα1β2 Fusions

Subsequently, LTα1β2 antibody fusions (FIG. 1G-1J and Table 2) comprising either 1 or 2 LTα1β2 moieties fused to the anti-EDB antibody EDBmAb1, were generated and tested in the reporter assay.

These constructs were designed in analogy to the work by Gurney et al. who reported in vitro and in vivo studies with a bispecific fusion construct consisting of a heterotrimeric single-chain LTα1β2 moiety fused to a B7-H4 specific tumor-targeting antibody (WO2018/119118). Importantly, unlike LIGHT used in the previous section, the LTα1β2 fusion constructs are specific agonists of LTBR and do not activate HVEM.

FIGS. 3C and 3D show the results obtained with COVA14113, the fusion of 2 LTα1β2 to the EDBmAb1 antibody (FIG. 1H and Table 2), COVA14116, the fusion of 1 LTα1β2 to the EDBmAb1 antibody (FIG. 1I and Table 2) compared to COVA14114, the fusion of 2 LTα1β2 to the isotype control antibody B21M (FIG. 1G and Table 2) that serves as non-targeted LTα1β2 control, soluble recombinant human LIGHT and to soluble LTα1β2 (recombinant human Lymphotoxin α1β2; Cat. No. 8884-LY/CF; R&D Systems). In the presence of EDB (FIG. 3C), both COVA14113 and COVA14116 achieved more potent activation of LTBR than the soluble natural ligands LIGHT and LTα1β2, however the non-targeted LTα1β2 control COVA14114 showed comparable activation levels as COVA14113 and COVA14116. In the absence of EDB (FIG. 3D), the activity of COVA14113 and COVA14114 (carrying two LTα1β2 moieties) was unchanged, whereas the activity of COVA14116 (carrying one LTα1β2 moiety) was reduced to a level slightly below the activation achieved by soluble LTα1β2. These data showed that tumor antigen-dependent activation of LTBR is very difficult to achieve with such antibody-LTα1β2 constructs. In fact, the activation levels that were achieved in the absence of EDB containing Fibronectin (FIG. 3D) could be problematic due to the broad expression of LTBR in normal tissue (Lukashev, et al. Cancer Res., 66(19):9617-24(2006).

### Bispecific Antibodies Based on Agonistic Anti-LTBR Antibodies

In order to achieve tumor antigen-dependent activation of LTBR, we set out to generate bispecific antibodies (1:1 heterodimers; we included KiH mutations in the Fc region to facilitate correct pairing) consisting of the anti EDB antibody EDBmAb1 and the anti LTBR agonistic antibodies LTBRmAb1 and LTBRmAb2 (FIGS. 1K-1M), with the aim of activating LTBR only upon binding to the tumor antigen (the tumor antigen being EDB of fibronectin, which is a tumor antigen present in the extracellular matrix). Corresponding control antibodies consisting of the isotype control antibody B21M paired with LTBRmAb1 and LTBRmAb2 (FIGS. 1N-10) were also generated.

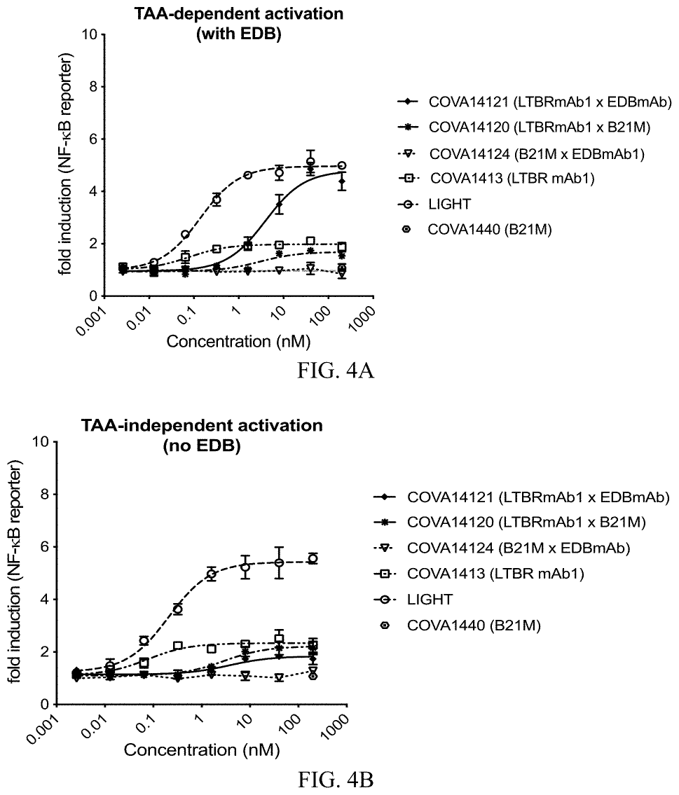

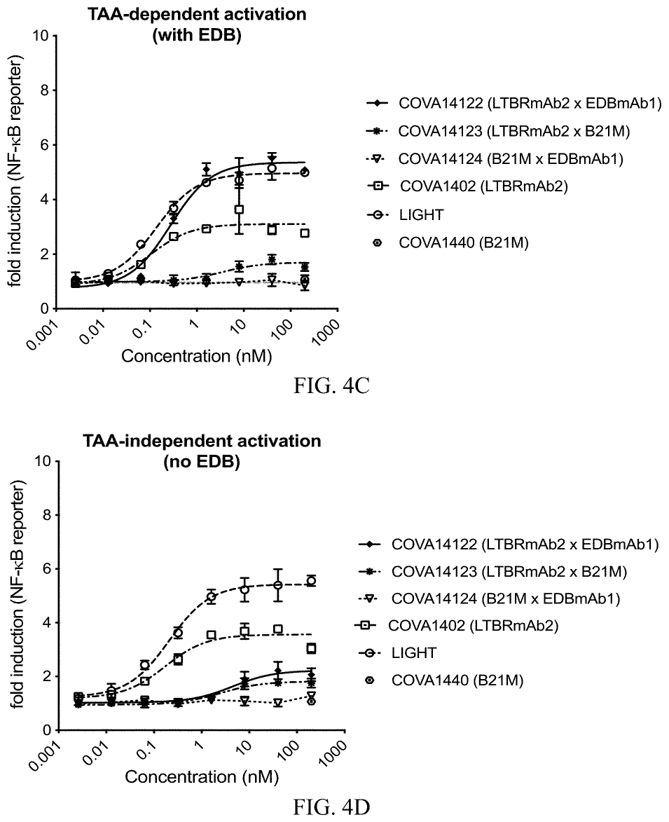

FIGS. 4A and 4C show that COVA14121 (1:1 heterodimer EDBmAb1 and LTBRmAb1; FIG. 1L and Table 2) and COVA14122 (1:1 heterodimer EDBmAb1 and LTBRmAb2; FIG. 1M and Table 2) were able to activate LTBR in an EDB dependent way. In contrast to molecules previously described, and in the examples above, as shown in FIGS. 4B and 4D, in the absence of EDB, COVA14121 and COVA14122 demonstrated only minimal LTBR activation. This residual activity could be due to residual impurities in the purified material. Table 5 shows a comparison of maximal fold inductions (of NF-kB signaling in presence or absence of ED-B containing Fibronectin) obtained with the heterodimers COVA14121 and COVA14122 or with LIGHT-(COVA1454) or LTα1β2-antibody fusions (COVA14113 and COVA14116). The comparison clearly shows, that using an agonistic antibody makes the LTBR bispecific molecules more specific. In fact, the ratio between maximal fold induction achieved in the presence of ED-B to the maximal fold induction achieved in absence of ED-B for COVA14121 and COVA14122 ranges between 4.4. and 5.4, whereas for the ligand-antibody fusions it ranges between 1.1 and 1.6, demonstrating, that these ligand-antibody fusions do not achieve specific TAA-dependent LTBR activation, in contrast to the bispecific antibodies of the present invention.

TABLE 5
Maximal fold inductions of NF-kB signaling
in presence or absence of ED-B

| | | | Ratio (max fold |
|---|---|---|---|
| | Max. fold | Max. fold | induction ED-B⁺)/ |
| | induction in | induction in | max fold induction |
| Name | presence of ED-B | absence of ED-B | ED-B⁻) |
| COVA14121 | 4.8 | 1.1 | 4.4 |
| COVA14122 | 5.4 | 1.0 | 5.4 |
| COVA1454 | 7.6 | 5.6 | 1.4 |
| COVA14113 | 6.7 | 5.9 | 1.1 |
| COVA14116 | 6.4 | 3.9 | 1.6 |

Taken together, these results suggested, that it was possible to activate LTBR in a tumor dependent way with minimal to no activation in the absence of tumor antigen using bispecific antibodies based on agonistic LTBR antibodies. In such bispecific molecules the LTBR binding antibody activates LTBR only upon binding to the tumor antigen, in this case EDB containing Fibronectin.

To further enhance the tumor antigen-dependent LTBR activation, a 2:1 bispecific format with 2 binding sites for EDB (to increase antigen mediated clustering) or 2 non-specific binding sites, and 1 binding site to LTBR (see FIGS. 1P-1W and FIGS. 1A2-1A5) was designed. For the LTBR binding site scFv fragments were used. As scFv fragments could have stability problems, 2 different methods were used to stabilize them, scFv fragments derived from LTBRmAb1 were stabilized using additional disulfide bonds between VH and VL (Reiter, et al., Nat Biotechnol. 14(10):1239-45 (1996)) or using the stapled scFv platform (VH-VL; VL-VH) and fused via a (G₄S)₃ linker to either EDBmAb1 or B21M (isotype control antibody).

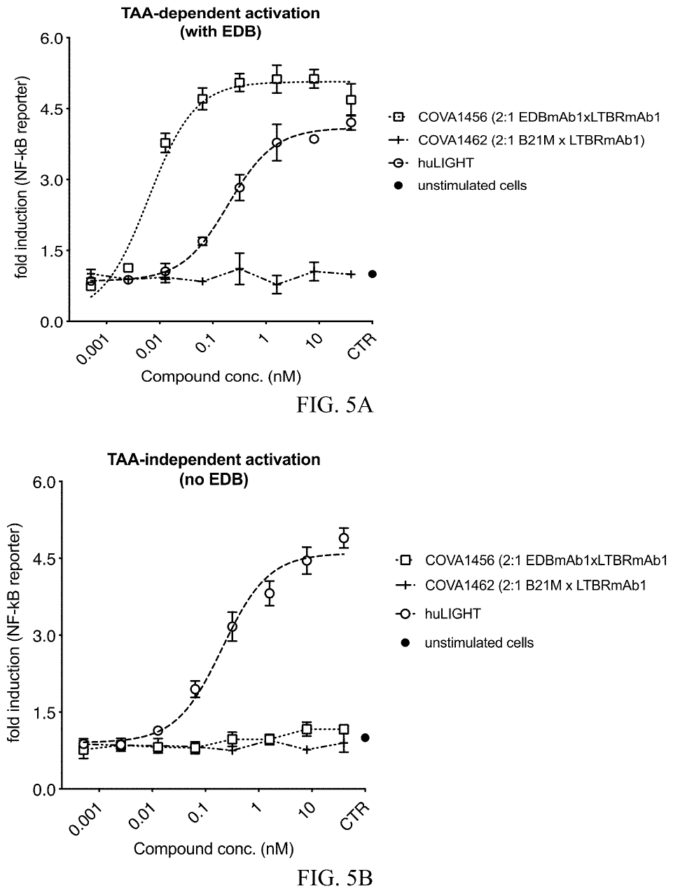

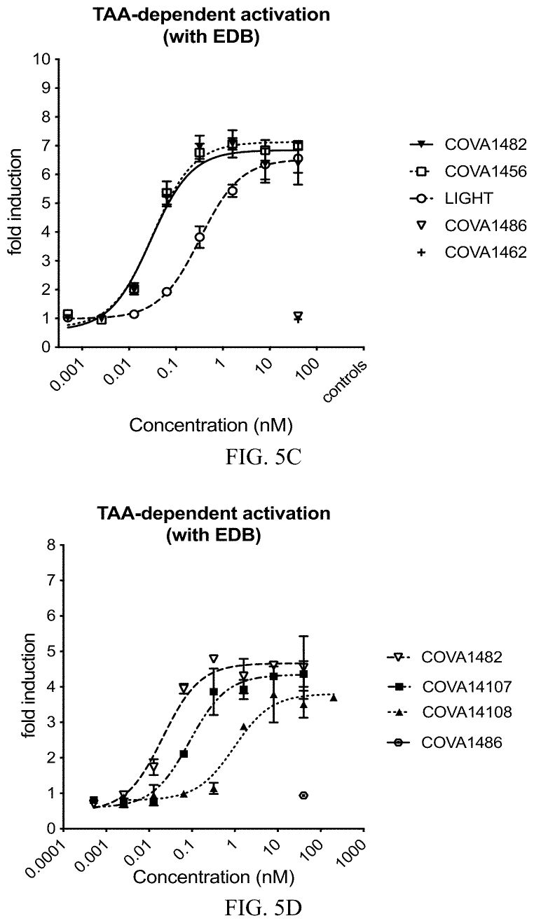

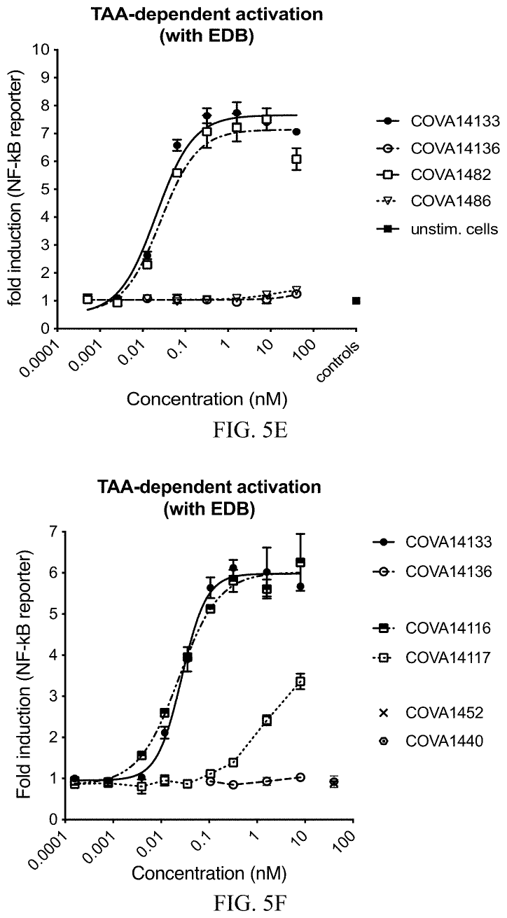

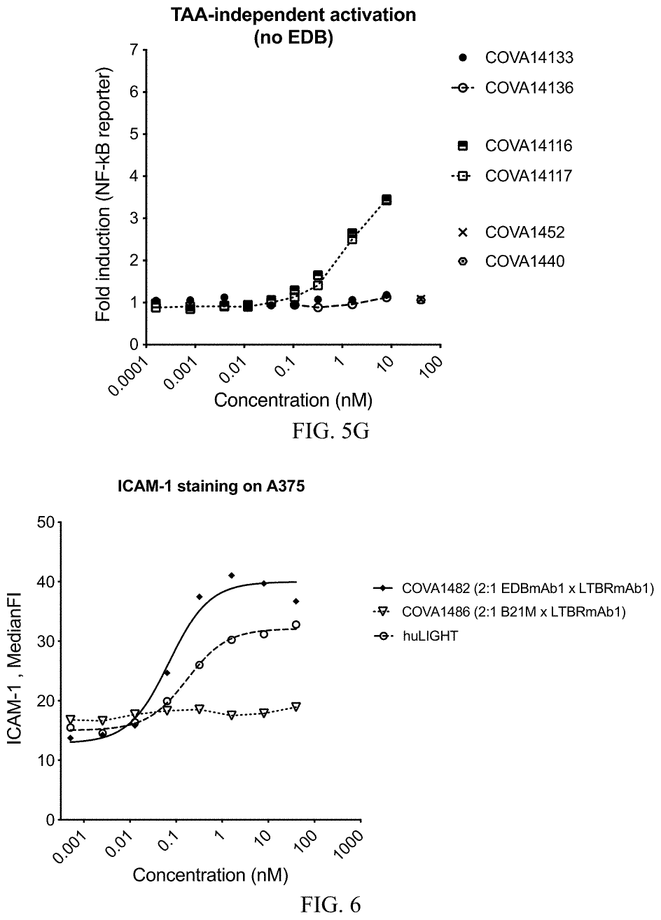

FIG. 5A demonstrated that COVA1456 (FIG. 1A4 and Table 2), a 2:1 bispecific EDB/LTBR antibody potently activated LTBR, whereas the control bispecific antibody COVA1462 (FIG. 1A5 and Table 2) was unable to activate LTBR. This indicated that clustering via binding to TAA (in this case immobilized EDB-containing Fibronectin) was a prerequisite for potent LTBR activation by the 2:1 bispecific antibody COVA1456. COVA1456 was not able to activate LTBR if EDB-containing Fibronectin was absent (FIG. 5B), which supported the fact that EDB presence was essential for LTBR activation and tumor specific activation of LTBR was achieved by bispecific antibodies targeting LTBR and EDB in the extracellular matrix.

In order to demonstrate, that the ability to activate LTBR in a TAA-dependent fashion was not an intrinsic property of the disulfide stabilized scFv derived from LTBRmAb1 used for the construction of COVA1456, COVA1456 was compared in the same A549 NF-kB reporter assay to COVA1482. COVA1482 differs from COVA1456 only in the stabilization method used for the scFv. The scFv in COVA1482, which was also derived from LTBRmAb1, was stabilized using the stapled platform. FIG. 5C showed that both COVA1482 and COVA1456 potently activated LTBR in an EDB dependent way. The corresponding isotype controls COVA1486 and COVA1462 did not activate LTBR (FIG. 5C). These results suggested that the method used to stabilize the scFv fragment did not influence the ability of the bispecifics to activate LTBR in a TAA-dependent manner. Surprisingly, the 2:1 bispecific EDB/LTBR antibodies (COVA1482 or COVA1456) showed increased potency in inducing NF-kB signaling in this reporter assay. The average EC₅₀ calculated for COVA1482 over several assays with the same experimental set up is of ca. 30 pM±10 pM, whereas COVA14121 (1:1 heterodimer) shows an EC₅₀ of ca. 3 nM in the assay shown in FIG. 4A, indicating that the 2:1 bispecifics can be 100 times more potent than 1:1 bispecifics. This could be explained by increased clustering of the LTBR binding site achieved with 2 binding sites to the TAA.

To study the effects of affinity to LTBR on the ability of such bispecifics to TAA-dependently activate LTBR, lower affinity variants (SEQ ID NO: 53, KD≈60 nM and SEQ ID NO: 54, KD≈600 nM) of the scFv fragment derived from LTBRmAb1 were generated and used to construct 2:1 bispecifics (COVA14107 FIG. 1X and Table 2; and COVA14108 FIG. 1Y and Table 2). The bispecifics were tested in the A549 NF-kB reporter assay to see the effects of affinity on activation of LTBR. FIG. 5D showed that lower affinity to LTBR corresponded to lower ability of the bispecific to activate LTBR in a TAA-dependent manner in this assay.

As mentioned in Example 1, mutations (WO2010151792) to abrogate binding to protein A (used for purification of antibodies) were introduced in the Fc of some constructs in order to facilitate the purification of the desired heterodimer. COVA14133 was generated without these mutations, and its activity was compared to COVA1482 to show that the mutations in the Fc region did not influence the activity of the bispecific. COVA14133 and COVA1482 and their respective isotype controls COVA14136 and COVA1486 were compared in the A549 NF-kB reporter assay. FIG. 5E showed that COVA14133 activated LTBR in a TAA-dependent manner with a similar efficiency as COVA1482, demonstrating that the mutations in the Fc did not influence the ability of the bispecific to activate LTBR.

In the presence of EDB (FIG. 5F), both COVA14133 (2:1 EDBmAb1×LTBR mAb1) and COVA14116 (2:1 EDBmAb1×LTα1β2) achieved potent activation of LTBR. No LTBR activation was observed with the non-targeted isotype control molecule COVA14136 (2:1 B21M x LTBR mAb1), however, the non-targeted LTα1β2 control COVA14117 (2:1 B21M×LTα1β2) showed activation independent of TAA binding. In the absence of EDB (FIG. 5G), no LTBR activation could be detected by COVA14133 or its isotype control molecule COVA14136. In contrast, TAA-independent activation of LTBR by COVA14116 and COVA14117 was measured in the absence of EDB, showing that tumor antigen-dependent activation of LTBR is very difficult to achieve with such antibody-LTα1β2 constructs.

In conclusion, COVA14133 was shown to have excellent ability to activate LTBR in a TAA-dependent manner.

### Example 3: EDB Dependent In Vitro LTBR Activation—A375/WI38VA Subline2Ra Co-Culture Cell Assay

The A375/WI38VA subline2RA co-culture assay was performed to verify if activation of LTBR in the presence of EDB+Fibronectin (produced and deposited in the extracellular matrix by WI38VA cells (Zardi, L., et al, EMBO J, 6, 2337-42 (1987)) leads to the release of cytokines and chemokines and upregulation of the adhesion molecule ICAM-1 on the A375 cells. WI38VA subline2RA (ATCC® CCL75.1™) cells were seeded in a 96-well plate at a density of 5000 cells/well and incubated for 48 hours in their growth medium (MEM w/o Glutamine+10% heat inactivated FBS+0.1 mM NEAA+2 mM L-Gln+1 mM Sodium pyruvate) at 37° C./5% CO₂. A 1:5 dilution series in triplicates of the compounds to be tested was prepared in assay medium (DMEM+10% heat inactivated FBS) as 2-fold concentration stocks (final concentrations tested ranged from 40 nM to 0.5 pM). Prior to incubation in the co-culture with the WI38VA subline2RA cells, A375 cells (ATCC® CRL-1619™) were labeled with CellTrace violet (CTV, Invitrogen; Carlsbad, Calif.). For labeling, a cell suspension, with a concentration of 10×10⁶ cells/ml and 2.5 μM CTV in 5% FBS in PBS, was incubated for 5 minutes at RT while protected from light. Cells were then washed and resuspended in assay medium at a density of 0.4×10⁶ cells/ml. Careful removal of culture medium from the plate containing the 48 hours WI38VA subline2RA culture, was followed by addition of 50 μl A375 cell suspension (20,000 cells/well; CTV+ or CTV−) in each well. 50 μl of the serial diluted compounds (final volume per well 100 μl) were added to the cells and incubated 24 hours at 37° C./5% CO₂.

After incubation for 24 or 72 hours, the supernatants were cleared by centrifugation and stored for measurement of cytokines and chemokines using MSD assays, or for use in a PBMC migration assay (24 hours incubation, Example 5). The cells were further processed for ICAM-1 measurement by flow cytometry (24 hours incubation).

Detection of ICAM-1 by Flow Cytometry

Any media left in the 96-well plate was carefully removed, cells were detached with Accutase, transferred to a DeepWell 96-well plate (triplicates were pooled in 1 well), washed, resuspended in 100 μl FACS buffer (PBS+1% FBS+0.1% NaN₃) and transferred to a round bottom 96-well plate. Antibody, i.e., anti-human ICAM-1 PE labeled (clone 1H4, Thermo; Waltham, Mass.) or isotype control antibody PE labeled (MPC-11, BioLegend; San Diego, Calif.) and LIVE/DEAD fixable near-IR stain (Invitrogen), single staining or combination staining was diluted as shown in Table 6.

TABLE 6
Dilution scheme of single staining
or combo staining in FACS buffer

| | Antibody | Final concentration | Dilution |
|---|---|---|---|
| | or stain | used (μg/ml) | factor |
| Single stainings | ICAM-1 | 1.25 | 20 |
| | Isotype control | 1.25 | 160 |
| | LIVE/DEAD | — | 400 |
| Combo staining | ICAM-1 | 1.25 | 20 |
| ICAM-1 + LIVE/DEAD | LIVE/DEAD | — | 400 |

Cells were centrifuged at 400×g at 4° C. for 4 minutes, the supernatant was discarded, and 50 μl antibody solutions were prepared as described in Table 6. Cells and antibodies were incubated in the dark at 4° C. for 30 minutes. After incubation, 120 μl were added in each well, and the cells were then centrifuged at 400×g at 4° C. for 4 minutes. Cells were washed once with FACS buffer, centrifuged and resuspended in 90 μl FACS buffer. Cells were then fixed by adding 90 μl of a 3.7% Formalin solution in PBS and were incubated for 15 minutes on ice in the dark. After fixation, cells were centrifuged at 400×g at 4° C. for 4 minutes and resuspended in 100μl FACS buffer. Cells were measured using a MACS Quant instrument at a high flowrate in screen mode, 49 μl/well were acquired. Data were analysed using the FlowLogics Software (Version 700.2A) and plotted with GraphPad Prism.

Cytokine Measurement in the Supernatants of Treated Cells Using MSD Platform

Several cytokines that are known to be under the control of NF-κB signaling were measured using the MSD platform and multiplex MSD plates. Listed here are some examples of measured cytokines:
RANTES: using R-Plex Antibody Set human RANTES (MSD);
I-TAC, IP-10, MIP-3b: using 3-PLEX cytokine release assay (MSD);
IL-8, IP-10, MIP-3b: using 3-PLEX cytokine release assay (MSD); and
IL-12p70, IL-6, TNF-α, MIP-3a, SDF-1a: using 5-PLEX cytokine release assay (MSD) The concentration of cytokines in the supernatant of treated cells was measured using the MSD platform following the manufacturer's instructions. Briefly, the protocol involved following steps:

(1) Preparation of the plate involved coating the provided plate with the linker-coupled capture antibodies. Plates were incubated with shaking overnight at 2-8° C. On the following day, plates were washed with PBST (PBS plus 0.05% Tween-20) using a plate washer (Biotek; Winooski, Vt.);

(2) Preparation of calibrator standard and detection antibody solution;

(3) Supernatants were diluted 1:3 or 1:5 depending on availability of material. Supernatants after 24 hours or 72 hours (for I-TAC, MIP-3a, TNFα) incubation were measured.

Assay Protocol:
Step 1: The sample or calibrator standard was added to the plate, and the palte was incubated at RT for 1 hour while shaking;
Step 2: The plates were washed, and the detection antibody was added. The plates were incubated with shaking for 1 hour at RT
Step 3: The plates were washed and 2× read buffer T was added. The plate was analyzed on an MSD instrument
Data were analyzed using Mesoscale Software (MSD discovery work bench program v 4.0.12.1). Dose response curves, including standard deviations from triplicates, were plotted using GraphPad Prism, and non-linear fits were applied (log(agonist) vs. response (variable slope−four parameters)), if applicable. In order to fit the data, the x-values (concentrations of compounds) were transformed using the X=Log(x) function of GraphPad Prism.

Results—Detection of ICAM-1 by Flow Cytometry

It was previously shown that NF-κB signaling can lead to upregulation of ICAM-1 on the surface of cells (da Silva Antunes, et al. Front Immunol, 9, 576, (2018)). Therefore, the levels of ICAM-1 expression on the surface of A375 cells after co-culture incubation with EDB/LTBR bispecifics were measured. As an example, FIG. 6 shows the upregulation of ICAM-1 after incubation with the EDB/LTBR bispecific COVA1482. In contrast, the isotype control molecule COVA1486 did not cause upregulation of ICAM-1. These findings indicated that the ability to cluster the LTBR scFv via binding to EDB was a prerequisite for LTBR activation, and as a consequence, ICAM-1 upregulation.

Results—Cytokine Measurement in the Supernatants of Treated Cells

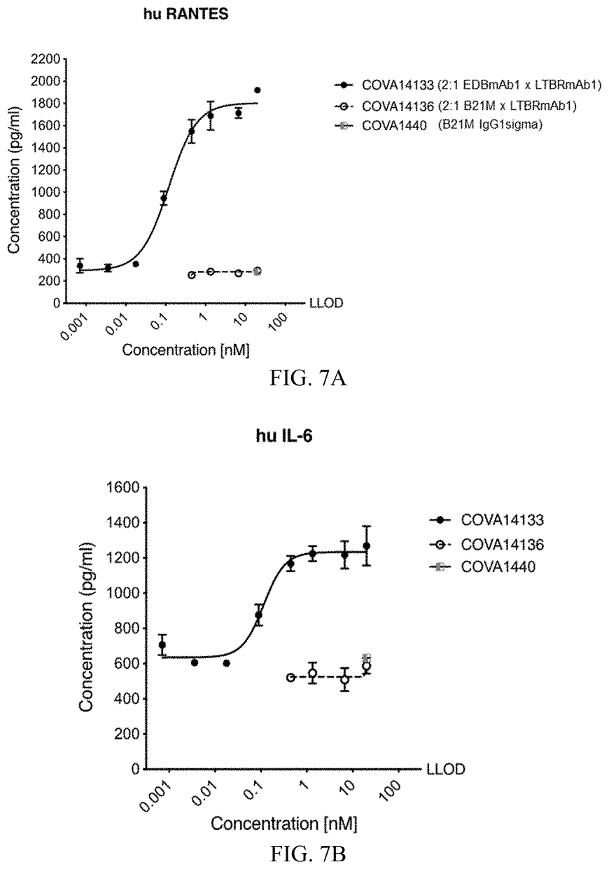

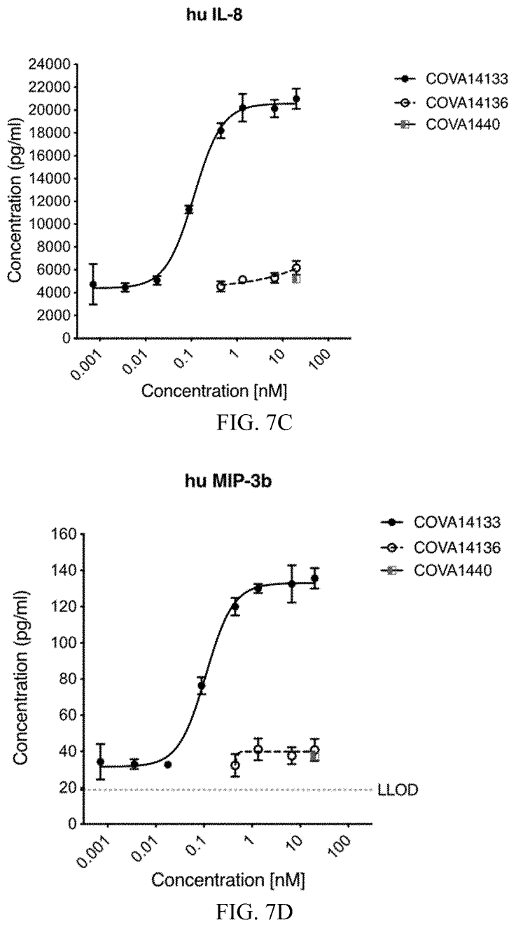

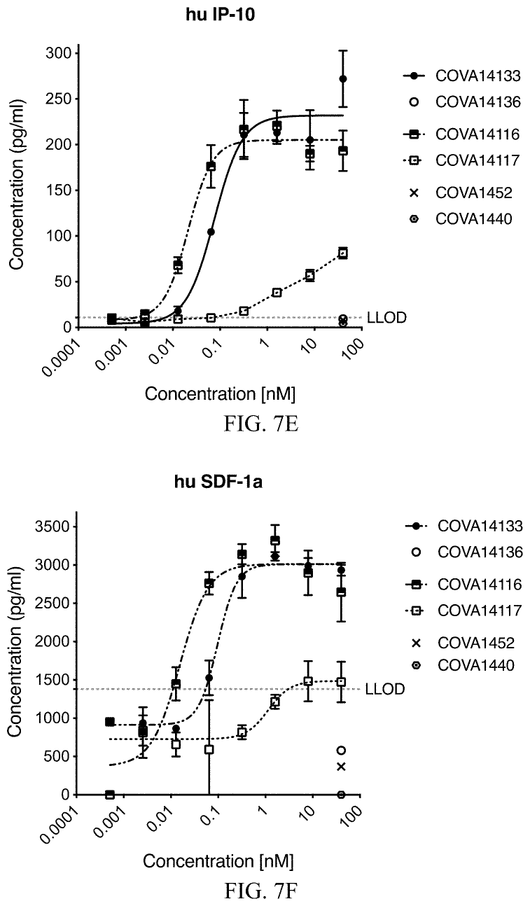

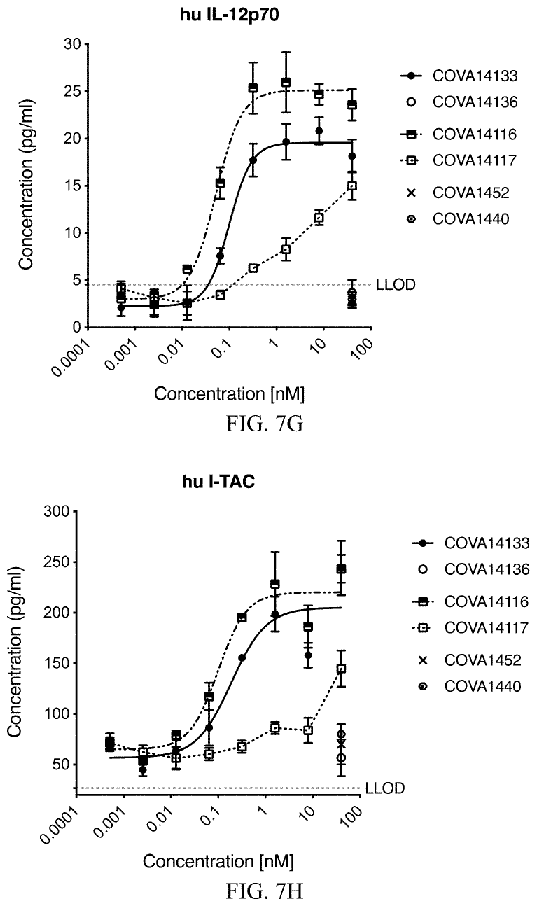

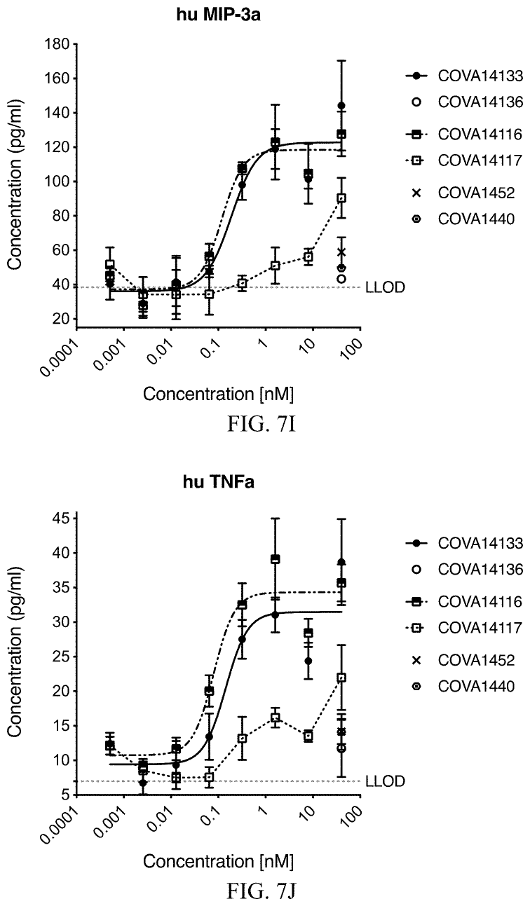

Several cytokines and chemokines, that are expressed as a result of LTBR activation were measured in the supernatant of the co-cultures that were treated with EDB/LTBR bispecifics and control molecules as described above. FIGS. 7A-J shows representative examples of cytokines readouts (FIG. 7A: RANTES, FIG. 7B: IL-6, FIG. 7C: IL-8, FIG. 7D: MIP-3b, FIG. 7E IP-10, FIG. 7F: SDF-1a, FIG. 7G: IL-12p70, FIG. 7H: I-TAC, FIG. 7I: MIP-3a, FIG. 7J: TNFα) that were upregulated by activation of LTBR with COVA14133. The untargeted LTBRmAb1 derived scFv in COVA14136 did not activate LTBR, and as a consequence, the concentration of cytokines in the supernatant was not increased above background. The background was represented by the level achieved with the B21M (COVA1440) and EDBmAb1 (COVA1452) antibodies, shown as a single concentration in the plots. FIGS. 7E-J show that both COVA14133 (2:1 EDBmAb1×LTBR mAb1) and COVA14116 (2:1 EDBmAb1×LTα1β2) achieved potent activation of LTBR, measured by induction of cytokine release. No LTBR activation was observed with the non-targeted isotype control molecule COVA14136 (2:1 B21M×LTBR mAb1), however, the non-targeted LTα1β2 control COVA14117 (2:1 B21M×LTα1β2) showed induction of cytokines independent of TAA binding. Again these data exemplify that tumor antigen-dependent activation of LTBR is very difficult to achieve with such antibody-LTα1β2 constructs.

Taken together, ICAM-1 upregulation and cytokine secretion upon LTBR activation confirmed the expected effects on cells that LTBR activation can have.

In this example, it was demonstrated that the molecules of this invention achieved efficient tumor associated antigen (EDB-containing Fibronectin) dependent activation of LTBR. Due to the broad expression of LTBR in normal tissue (Lukashev, et al. Cancer Res., 66(19):9617-24(2006)), the molecules of this invention have a clear advantage over previously described LIGHT and LTα1β2 antibody fusions, since such previously described fusions were shown herein to efficiently activate LTBR also in the absence of the tumor associated antigen, and thus lack the desired tumor specificity for LTBR activation. In contrast, the multispecific binding molecules of the invention surprisingly do have this desired tumor specificity.

### Comparative Example 4: Mesothelin Dependent In Vitro LTBR Activation—Co-Culture Cell Assay with A549 NF-κB Reporter Cells and CHOK1-huMSLN or 11226

In the Examples 2 and 3, it was demonstrated that bispecific antibodies, targeting EDB (a tumor associated antigen in the extracellular matrix) and LTBR, activated LTBR very efficiently in a tumor antigen dependent way. In order to verify if this finding holds true for any tumor antigen despite its location (deposited in the extracellular matrix or on the cell surface of tumor cells), a bispecific 2:1 antibody targeting Mesothelin (MSLN), a tumor associated antigen expressed on different types of tumor (Hassan and Ho, European Journal of Cancer, 44: 46-53 (2008)) and LTBR was designed and produced as described in Example 1. COVA14146 is a 2:1 MSLN/LTBR bispecific antibody consisting of an anti-Mesothelin antibody (MSLNmAb1) fused to a scFv fragment derived from LTBRmAb1. To show if a LTBR bispecific antibody targeting LTBR and a tumor associated antigen present of the cell surface of tumor cells (e.g. Mesothelin) was able to efficiently activate LTBR in a tumor-dependent way, a co-culture cell assay was used. The co-culture assays used were the A549 cell NF-κB luciferase reporter cell assay (described in Example 2) and H226 cells (mesothelioma cell line; ATCC® CRL-5826) known to express Mesothelin (Fan et al. Mol. Canc. Ther. Vol. 1, 595-600 (2002)) and LTBR.

### Preparation of 11226 Cells

10,000 cells per well (in 75 μl assay medium: DMEM+10% FBS-HI) of a H226 cell (express about 200,000 copies of Mesothelin and 10,000 copies of LTBR) suspension were seeded in a 96-well tissue culture plate and were incubated for 6 hours at 37° C./5% CO₂ in their growth media (MEM+2 mM Glutamine+10% FBS-HI+10 μg/ml Puromycin and RPMI-1640+10% FBS+1 mM Na-Pyruvate respectively) to allow the cells to attach to the plate.

### Preparation of Compounds

The compounds were tested in a concentration range from 100 nM down to 1.3 pM. A 4-fold 1 in 5 serial dilution of the compounds was prepared in assay medium (DMEM+10% FBS-HI) and stored at 4° C. until use.

### Preparation and Addition of A549 Reporter Cells

A549 reporter cells were detached from the cell culture flask with Accutase/EDTA and transferred in to assay medium (DMEM+10% FBS-HI). After adding a total of 20′000 A549 reporter cells per well to the plates containing H226 cells, 50 μL of the pre-diluted compounds were added to each well and incubated for 20 hrs at 37° C./5% CO₂.

### Measurement of Luminescence in Treated Co-Cultures

After incubation for 20 hours, the Bio-Glo™ Luciferase Assay System (Promega; Madison, Wis.) was used according to manufacturer's instructions to detect luciferase activity. Luminescence was measured using a Tecan M1000 Pro instrument with an integration time of 500 milliseconds. From the resulting relative light units (RLU), the fold induction of LTBR signaling was calculated as follows: Fold induction=RLU_{stimulated} cells/average RLU_{unstimulated} cells (unstimulated cells were included as control in each plate tested).

Dose response curves, including standard deviations, were plotted using GraphPad Prism, and non-linear fits were applied (log(agonist) vs. response (variable slope−three parameters)), if applicable. In order to fit the data, the x-values (concentrations of compounds) were transformed using the X=Log(x) function of GraphPad Prism.

### Cytokine Measurement in the Supernatants of Treated Cells Using MSD Platform

Several cytokines known to be under the control of NF-κB signaling can be measured using the MSD platform and multiplex MSD plates. As an example, the method for the measurement of RANTES using R-Plex Antibody Set human RANTES (MSD) is described herein.

The concentration of RANTES in the supernatant of treated cells was measured using the MSD platform following the manufacturer's instructions. Briefly, the protocol involved following steps:

(1) Preparation of the plate involved coating the provided plate with the linker-coupled capture antibodies. Plates were incubated with shaking overnight at 2-8° C. On the following day, plates were washed with PBST (PBS plus 0.05% Tween-20) using a plate washer (Biotek; Winooski, Vt.);

(2) Preparation of calibrator standard and detection antibody solution;

(3) Supernatants were diluted 1:3 or 1:5 depending on availability of material.

Assay Protocol:
Step 1: The sample or calibrator standard was added to the plate, and the plate was incubated at RT for 1 hour while shaking;
Step 2: The plates were washed, and the detection antibody was added. The plates were incubated with shaking for 1 hour at RT
Step 3: The plates were washed and 2× read buffer T was added. The plate was analyzed on an MSD instrument
Data were analyzed using Mesoscale Software (MSD discovery work bench program v 4.0.12.1) and plotted using GraphPad Prism.

### Results—Mesothelin-Dependent Activation of LTBR in A549 Reporter Cells/H226 Co-Culture Assay

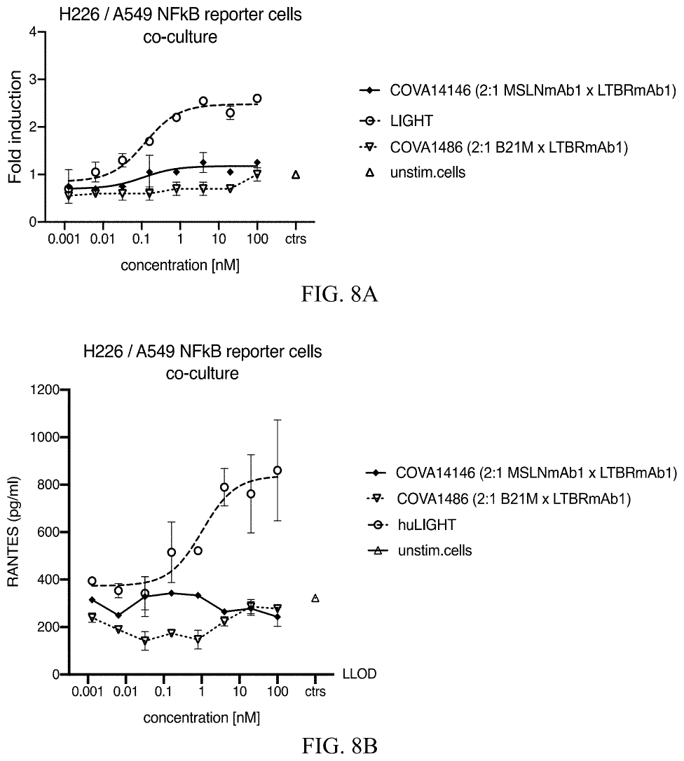

A co-culture assay with A549 reporter cells and H226 cells was performed to verify if COVA14146 was able to activate LTBR in a more physiological system, where, due to its broad expression (Lukashev, et al. Cancer Res., 66(19):9617-24(2006), LTBR and Mesothelin (and other tumor associated antigens on the cell surface of tumor cells, e.g. EGFR) are expected to be co-expressed on the cell surface of tumor cells. In FIG. 8A, it was shown that under these conditions COVA14146 did not activate LTBR efficiently. The concentrations of RANTES secreted in the supernatants of treated cells were measured to confirm the inability of COVA14146 to efficiently activate LTBR. As expected, FIG. 8B shows that RANTES was secreted by cells treated with COVA14146 to same extent as from cells that were treated with the isotype control molecule COVA1486, confirming that LTBR cannot be activated under these conditions.

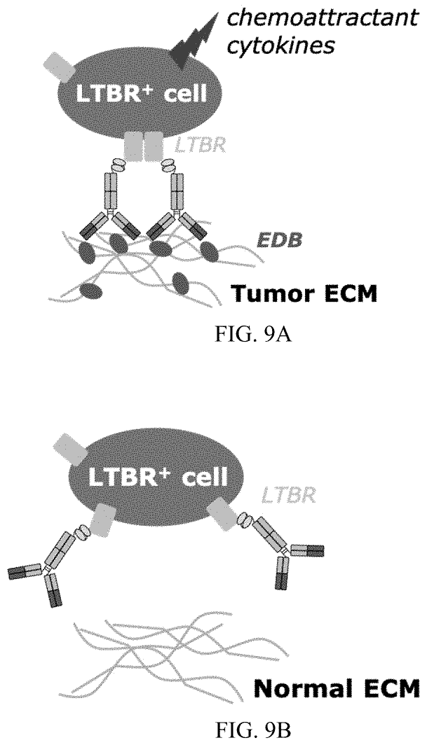

Taken together, the data presented in Examples 2-4 suggested, that EDB-containing Fibronectin (a tumor associated antigen that is deposited in the extracellular matrix of a tumor; FIG. 9A), can lead to efficient clustering of LTBR leading to its efficient activation unlike antigens co-expressed with LTBR on the surface of tumor cells (e.g. Mesothelin). Activation of LTBR under the conditions depicted in FIG. 9A, by the bispecific antibodies of this invention, led to secretion of chemoattractant chemokines and cytokines and the overexpression of adhesion molecules (e.g. ICAM-1) on treated cells. In the absence of EDB-containing Fibronectin (FIG. 9B) the bispecific antibodies of this invention were not able to activate LTBR and as a consequence no expression of chemoattractant chemokines and cytokines or overexpression of adhesion molecules was observed. Moreover, it was shown that tumor associated antigens co-expressed with LTBR on tumor cells were not suitable to activate LTBR in a tumor-dependent manner by bispecific antibodies.

In short, it is shown herein that targeting of LTBR and a tumor-associated antigen (TAA) that is co-expressed with LTBR on tumor cells with a bispecific antibody that binds to both LTBR and such TAA, was not capable of activating LTBR in a tumor-specific manner (example 4), and that targeting of LTBR via fusion proteins that contain a TAA-binding part and one of the natural LTBR ligands LIGHT or LTα1β2 did result in LTBR activation but not in a tumor-specific manner (example 2). Strikingly and surprisingly however, bispecific antibodies that bind with one binding domain to LTBR and with another binding domain to EDB of Fibronectin (a TAA that is present in the extracellular matrix), were capable of activating LTBR in a tumor-specific manner (examples 2 and 3). Particularly good results were observed when such bispecific antibodies contained three binding domains, e.g. two binding domains targeting the EDB and one binding domain targeting LTBR. This makes the bispecific antibodies of the invention interesting candidates for cancer immunotherapy, in view of their tumor specificity.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

### Example 5: Transwell Migration of PBMC Towards Conditioned Medium from A375/WI38VA Subline2RA Co-Culture Cell Assay

In Example 3, it was demonstrated that bispecific antibodies, targeting EDB (a tumor associated antigen in the extracellular matrix) and LTBR, activated LTBR very efficiently in a tumor antigen dependent way, resulting in the production of pro-inflammatory cytokines.

The aim of this assay is to study if the cytokines and chemokines, produced in the co-culture assay, can attract PBMCs and lead to their migration. Human PBMCs were isolated from buffy coats by Ficoll Paque density gradient centrifugation and A375/WI38VA co-cultures were prepared and stimulated with EDB/LTBR bispecifics and control molecules as described in Example 3.

After 24 hrs incubation at 37° C./5% CO₂ the supernatants of the stimulated co-cultures were transferred to 96-well DeepWell plates and were diluted 1:1 with assay medium (RPMI1640+10% FBS+1 mM Sodium pyruvate). After dilution the supernatants were centrifuged (500×g/5 min) and transferred to a fresh 96-well DeepWell plate to eliminate any cells or cellular debris.

Recombinant SDF-1a, a potent chemoattractant, was used at a concentration of 40 ng/ml in assay medium as a positive control to stimulate PBMCs migration. 235 μl/well of the conditioned media, SDF-1a controls or assay medium were transferred for the migration assay (performed in triplicate) in the carrier plates of HTS Transwell®-96 Permeable Supports with 5 μm Pore Polycarbonate Membrane (Corning), that had been previously equilibrated in assay medium (RPMI1640+10% FBS+1 mM Sodium pyruvate) at 37° C./5% CO₂ for at least 1 hr. After placing the membrane inserts back into the carrier plates, 75 μl/well of a PBMC suspension with 4.67×10⁶ cells/ml were added to all wells of the migration assay plates, resulting in 350′000 cells/well. The plates were incubated for 2 hrs at 37° C./5% CO₂ to allow migration of PBMC towards the conditioned medium.

After 2 hrs incubation the plate inserts were removed and the migrated cells in the carrier plates were carefully resuspended and transferred to fresh U-bottom 96-well plates. The migrated cells were centrifuged (400×g/4 min), re-suspended in 50 FACS buffer (PBS containing 1% FBS-HI, 0.1% Natriumazide, 1 mM EDTA), and directly measured using a MACS Quant instrument (high flow rate and in fast mode). Data were analysed using the FlowLogics Software (Version 700.2A). Dose response curves, including standard deviations from triplicates, were plotted using GraphPad Prism. In order to fit the data, the x-values (concentrations of compounds) were transformed using the X=Log(x) function of GraphPad Prism.

Results—Transwell Migration of PBMC Towards Conditioned Medium from A375/WI38VA subline2RA Co-Culture Cell Assay

In this example it was studied if the cocktail of cytokines and chemokines, expressed upon activation of LTBR in a co-culture assay (see Example 3), could induce migration of PBMCs towards the cytokines and chemokines gradient. A transwell migration assay was established, where the supernatants of co-cultures stimulated with EDB/LTBR bispecific antibodies at different concentrations were placed in the lower chamber, whereas freshly isolated human PBMCs were added to the upper chamber of a transwell plate. After 2 hrs incubation time the migrated cells were counted and phenotyped by flow cytometry. FIG. 10 shows a representative result of a migration assay. The migration of PBMCs was induced in a dose-dependent manner towards supernatants from co-cultures stimulated with COVA14133 (EDB/LTBR bispecific), whereas supernatants from co-cultures incubated with non-targeted control molecule COVA14136 (isotype control/LTBR) did not induce migration of PBMCs.—LTα1β2 antibody fusions COVA14116 (EDB mAb1-LTα1β2; 2:1) and to some extend COVA14117 (B21M-LTα1β2; 2:1) did also induce migration of PBMCs. The migration of different immune cell sub-populations was confirmed by staining with immune cell markers to phenotype the migrated cells. The migration of monocytes, eosino-/neutrophils, basophils, NK cells, NKT cells, dendritic cells and T cells was confirmed (data not shown). This example confirms that EDB-dependent activation of LTBR leads to the secretion of cytokines and chemokines and shows that these can act as chemoattractant for immune cells (FIG. 10).

Moreover this example shows once again, that the molecules of this invention have a clear advantage over previously described LTα1β2 antibody fusions, since such previously described fusions were shown herein to efficiently activate LTBR also in the absence of the tumor associated antigen and lead to migration of PBMCs, and thus lack the desired tumor specificity for LTBR activation. In contrast, the multispecific binding molecules of the invention surprisingly do have this desired tumor specificity.

### Example 6: Effects of EDB-Dependent LTBR-Mediated Endothelial Activation on the Trafficking of Monocytes Through Endothelial Monolayers

After demonstrating in Examples 3 and 5, that the cytokines produced upon EDB-dependent activation of LTBR could lead to the migration of PBMC towards the cytokine gradient, the aim of the assay described here was to verify if the activation of LTBR on endothelial cells in an EDB-dependent manner would lead to increased trafficking of monocytes through the endothelial layer.

The monocytes used in this assay were purified from EDTA-blood collected from healthy donors using the respective negative selection kits (Miltenyi Biotec) and were used at 1.5×10⁶ cells/ml. HUVECs (human umbilical vein endothelial cells) were cultured in chamber slides coated with recombinant EDB+Fibronectin domains 7-B-8-9 (EDB+; SEQ ID NO: 51) for 48 hrs using M199 supplemented media (M199 media, 20% FCS, hydrocortisone (0.1 μM), heparin (100 μg/ml), ECGS 15 μg/ml, vitamin C (10 μg/ml), penicillin/streptomycin (1%/1%)). The HUVECs were then stimulated with TNF (500 U/ml; positive control), EDB/LTBR bispecific (COVA14133; 50 nM), or the untargeted LTBRmAb1 derived scFv (COVA14136; 50 nM) and were incubated for 2 days.

The flow assay set-up consisted of a heated microscope chamber (37° C.) and a calibrated pump where flow can be generated over HUVEC monolayers by perfusing wash buffer (M199 culture media, 0.1% BSA)+/−monocyte suspension. The flow rate is representative of small venules/capillaries (0.05-Pa). Wash-buffer is then pumped over the HUVECs for 10-mins to remove activation media, equating to 20-mins of total HUVECs exposure. Monocytes are then perfused over the HUVECs for 6-mins (Step-2) followed by 50-mins of wash-buffer (Step-3). This was done at 0.1-Pa, which is standard for all monocytes recruitment protocols. Throughout steps 2-3, images of the captured monocytes were made using phase-contrast microscopy, and a camera. Individual images were recorded every 30-secs on 1 fixed field and compiled into short movie sequences, allowing analysis of individual monocytes over large areas. Monocytes adherent to the surface of the HUVECs have a phase-white/grey appearance, whereas those that have transmigrated have a phase-black appearance.

The adherent and the transmigrated cells were counted within a fixed grid on each image in a defined area of 0.19 mm² for the duration of the experiment by playing the movie sequence. Time points for each cell count are conducted at fixed timepoints throughout the experiment. The total number of adherent cells is representative of the sum of captured cells at each time point; a percentage of which will transmigrate (Phase grey+black). Transmigration events (phase black) are a percentage of total monocytes (Phase grey+black) captured from flow per unit field. Monocytes typically remain adherent with very few detachment events for the duration of co-culture.

All experiments were carried out using triplicate fields and presented as a mean value with +standard error measurements (+SEM). Statistical analyses assumed parametric distributions and were conducted using the Student T test. P values from significance scores are presented on figures as follows: * P<0.05, ** P<0.01, *** P<0.005 (Bradfield P F, et al. Blood. 2007 Oct. 1; 110(7):2545-55).

### Results—Effects of EDB-Dependent LTBR-Mediated Endothelial Activation on the Trafficking of Monocytes Through Endothelial Monolayers

This example studies the effects of EDB-dependent LTBR activation on endothelial cell monolayers on the adhesion and transmigration of monocytes, as endothelial cells have been previously shown to express LTBR on their surface (Lukashev, et al. Cancer Res., 66(19):9617-24(2006)).

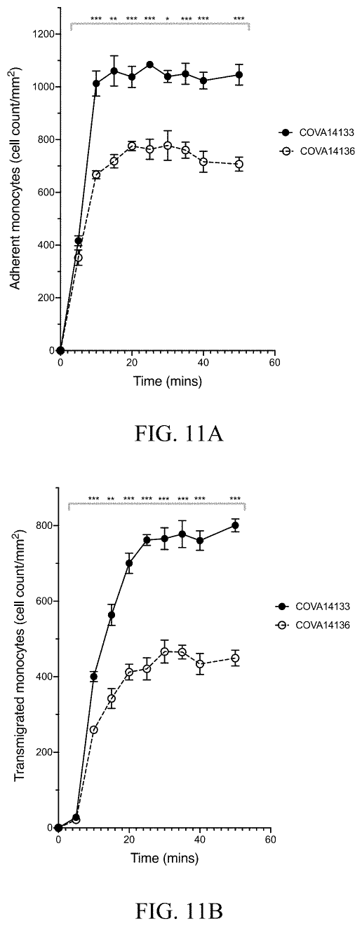

FIG. 11A shows that more monocytes can adhere to HUVEC monolayers, grown in presence of EDB-containing Fibronectin, that were activated with COVA14133 (EDB/LTBR bispecific) as compared with monolayers activated with the non-targeted control molecule COVA14136 (isotype control/LTBR).

FIG. 11B shows that, not only the adhesion, but also the transmigration of monocytes through the HUVEC monolayer after activation with COVA14133 is increased compared to HUVEC that were incubated with the non-targeted control COVA14136.

In conclusion, the results of this example further confirm, that the molecules of this invention have the clear advantage of being able to activate LTBR only in the presence of EDB-containing Fibronectin, unlike previously described LTα1β2 antibody fusions, providing the desired tumor specificity for LTBR activation.

SEQUENCE LISTING

SEQ ID NO: 1 (HC BHA10 IgG1s knob)
QVQLVQSGAEVKKPGSSVKVSCKASGYTFTTYYLHWVRQAPGQGLEWMGWIYPGNVHAQYNEKFKGRVTITADKSTS
TAYMELSSLRSEDTAVYYCARSWEGFPYWGQGTTVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTHTCPPCPAP
EAAGASSVFLFPPKPKDTLMISRTPEVTCVVVDVSAEDPEVKFNWYVDGVEVHNAKTKPREEQYNSTYRVVSVLTVL
HQDWLNGKEYKCKVSNKALPSSIEKTISKAKGQPREPQVYTLPPCREEMTKNQVSLWCLVKGFYPSDIAVEWESNGQ
PENNYKTTPPVLDSDGSFFLYSKLTVDKSRWQQGNVFSCSVMHEALHNHYTQKSLSLSPGK
SEQ ID NO: 2 (LC BHA10)
DIQMTQSPSSLSASVGDRVTITCKASQNVGINVAWYQQKPGKAPKSLISSASYRYSGVPSRFSGSGSGTDFTLTISS
LQPEDFATYFCQQYDTYPFTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
SEQ ID NO: 3 (HC L19 IgG1s knob)
EVQLLESGGGLVQPGGSLRLSCAASGETFSSFSMSWVRQAPGKGLEWVSSISGSSGTTYYADSVKGRFTISRDNSKN
TLYLQMNSLRAEDTAVYYCAKPFPYFDYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTHTCPPCPAP
EAAGASSVFLFPPKPKDTLMISRTPEVTCVVVDVSAEDPEVKFNWYVDGVEVHNAKTKPREEQYNSTYRVVSVLTVL
HQDWLNGKEYKCKVSNKALPSSIEKTISKAKGQPREPQVYTLPPCREEMTKNQVSLWCLVKGFYPSDIAVEWESNGQ
PENNYKTTPPVLDSDGSFFLYSKLTVDKSRWQQGNVFSCSVMHEALHNHYTQKSLSLSPGK
SEQ ID NO: 4 (HC L19 IgG1s hole)
EVQLLESGGGLVQPGGSLRLSCAASGETFSSFSMSWVRQAPGKGLEWVSSISGSSGTTYYADSVKGRFTISRDNSKN
TLYLQMNSLRAEDTAVYYCAKPFPYFDYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTHTCPPCPAP
EAAGASSVFLFPPKPKDTLMISRTPEVTCVVVDVSAEDPEVKFNWYVDGVEVHNAKTKPREEQYNSTYRVVSVLTVL
HQDWLNGKEYKCKVSNKALPSSIEKTISKAKGQPREPQVCTLPPSREEMTKNQVSLSCAVKGFYPSDIAVEWESNGQ
PENNYKTTPPVLDSDGS FELVSKLTVDKSRWQQGNVESCSVMHEALHNHYTQKSLSLSPGK
SEQ ID NO: 5 (LC L19)
EIVLTQSPGTLSLSPGERATLSCRASQSVSSSFLAWYQQKPGQAPRLLIYYASSRATGIPDRFSGSGSGTDFTLTIS
RLEPEDFAVYYCQQTGRIPPTFGQGTKVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC
SEQ ID NO: 6 (HC B21M (RSV) IgG1s knob)
QITLKESGPTLVKPTQTLTLTCTFSGFSLSTSGMGVSWIRQPPGKALEWLAHIYWDDDKRYNPSLKSRLTITKDTSK
NQVVLTMTNMDPVDTATYYCARLYGFTYGFAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTHTCPP
CPAPEAAGASSVFLFPPKPKDTLMISRTPEVTCVVVDVSAEDPEVKFNWYVDGVEVHNAKTKPREEQYNSTYRVVSV
LTVLHQDWLNGKEYKCKVSNKALPSSIEKTISKAKGQPREPQVYTLPPCREEMTKNQVSLWCLVKGFYPSDIAVEWE
SNGQPENNYKTTPPVLDSDGSFFLYSKLTVDKSRWQQGNVFSCSVMHEALHNHYTQKSLSLSPGK
SEQ ID NO: 7 (HC B21M (RSV) IgG1s hole)
QITLKESGPTLVKPTQTLTLTCTFSGFSLSTSGMGVSWIRQPPGKALEWLAHIYWDDDKRYNPSLKSRLTITKDTSK
NQVVLTMTNMDPVDTATYYCARLYGFTYGFAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTHTCPP
CPAPEAAGASSVFLFPPKPKDTLMISRTPEVTCVVVDVSAEDPEVKFNWYVDGVEVHNAKTKPREEQYNSTYRVVSV
LTVLHQDWLNGKEYKCKVSNKALPSSIEKTISKAKGQPREPQVCTLPPSREEMTKNQVSLSCAVKGFYPSDIAVEWE
SNGQPENNYKTTPPVLDSDGSFELVSKLTVDKSRWQQGNVESCSVMHEALHNHYTQKSLSLSPGK
SEQ ID NO: 8 [LC B21M (RSV)]
DIVMTQSPDSLAVSLGERATINCRASQSVDYNGISYMHWYQQKPGQPPKLLIYAASNPESGVPDRFSGSGSGTDFTL
TISSLQAEDVAVYYCQQIIEDPWTFGQGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGEC
SEQ ID NO: 9 HC (CBEll IgG1s knob)
EVQLVESGGGLVKPGGSLRLSCAASGETFSDYYMYWFRQAPGKGLEWVATISDGGSYTYYPDSVKGRFTISRDNAKN
SLYLQMSSLRAEDTAVYYCAREENGNFYYFDYWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTHTCPP
CPAPEAAGASSVFLFPPKPKDTLMISRTPEVTCVVVDVSAEDPEVKFNWYVDGVEVHNAKTKPREEQYNSTYRVVSV
LTVLHQDWLNGKEYKCKVSNKALPSSIEKTISKAKGQPREPQVYTLPPCREEMTKNQVSLWCLVKGFYPSDIAVEWE
SNGQPENNYKTTPPVLDSDGSFFLYSKLTVDKSRWQQGNVFSCSVMHEALHNHYTQKSLSLSPGK
SEQ ID NO: 10 (LCCBE11)
DIQMTQSPSSLSASVGDRVTITCKAGQDIKSYLSWYQQKPGKAPKLLIYYATRLADGVPSRFSGSGSGTDYTLTISS
LQPEDFATYYCLQHGESPWTFGGGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
SEQ ID NO: 11 (HC BHA10 IgG1s)
QVQLVQSGAEVKKPGSSVKVSCKASGYTFTTYYLHWVRQAPGQGLEWMGWIYPGNVHAQYNEKFKGRVTITADKSTS
TAYMELSSLRSEDTAVYYCARSWEGFPYWGQGTTVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTHTCPPCPAP
EAAGASSVFLFPPKPKDTLMISRTPEVTCVVVDVSAEDPEVKFNWYVDGVEVHNAKTKPREEQYNSTYRVVSVLTVL
HQDWLNGKEYKCKVSNKALPSSIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK
SEQ ID NO: 12 (HC L19 IgG1s)
EVQLLESGGGLVQPGGSLRLSCAASGETFSSFSMSWVRQAPGKGLEWVSSISGSSGTTYYADSVKGRFTISRDNSKN
TLYLQMNSLRAEDTAVYYCAKPFPYFDYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTHTCPPCPAP
EAAGASSVFLFPPKPKDTLMISRTPEVTCVVVDVSAEDPEVKFNWYVDGVEVHNAKTKPREEQYNSTYRVVSVLTVL
HQDWLNGKEYKCKVSNKALPSSIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK
SEQ ID NO: 13 (HCCBE11 IgG1s)
EVQLVESGGGLVKPGGSLRLSCAASGETFSDYYMYWFRQAPGKGLEWVATISDGGSYTYYPDSVKGRFTISRDNAKN
SLYLQMSSLRAEDTAVYYCAREENGNFYYFDYWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTHTCPP
CPAPEAAGASSVFLFPPKPKDTLMISRTPEVTCVVVDVSAEDPEVKFNWYVDGVEVHNAKTKPREEQYNSTYRVVSV
LTVLHQDWLNGKEYKCKVSNKALPSSIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLSLSPGK
SEQ ID NO: 14 (HC B21M (RSV) IgG1s)
QITLKESGPTLVKPTQTLTLTCTFSGFSLSTSGMGVSWIRQPPGKALEWLAHIYWDDDKRYNPSLKSRLTITKDTSK
NQVVLTMTNMDPVDTATYYCARLYGFTYGFAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTHTCPP
CPAPEAAGASSVFLFPPKPKDTLMISRTPEVTCVVVDVSAEDPEVKFNWYVDGVEVHNAKTKPREEQYNSTYRVVSV
LTVLHQDWLNGKEYKCKVSNKALPSSIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLSLSPGK
SEQ ID NO: 15 (3xhmLIGHT fusion to Fc with IgG1s, knob and pA mut)
RRSHEVNPAAHLTGANSSLTGSGGPLLWETQLGLAFLRGLSYHDGALVVTKTGYYYIYSKVQLGGVGCPLGLAGTIT
HGLYKRTPRYPEELELLVSQQSPCGRATSSSRVWWDSSFLGGVVHLEAGEKVVVRVLGKRLVRLRDGTRSYFGAFMV
GGGGSGGGGSGGGGSGGGGSRRSHEVNPAAHLTGANSSLTGSGGPLLWETQLGLAFLRGLSYHDGALVVTKTGYYYI
YSKVQLGGVGCPLGLAGTITHGLYKRTPRYPEELELLVSQQSPCGRATSSSRVWWDSSFLGGVVHLEAGEKVVVRVL
GKRLVRLRDGTRSYFGAFMVGGGGSGGGGSGGGGSGGGGSRRSHEVNPAAHLTGANSSLTGSGGPLLWETQLGLAFL
RGLSYHDGALVVTKTGYYYIYSKVQLGGVGCPLGLAGTITHGLYKRTPRYPEELELLVSQQSPCGRATSSSRVWWDS
SFLGGVVHLEAGEKVVVRVLGKRLVRLRDGTRSYFGAFMVGGGGSGGGGSGGGGSDKTHTCPPCPAPEAAGASSVFL
FPPKPKDTLMISRTPEVTCVVVDVSAEDPEVKFNWYVDGVEVHNAKTKPREEQYNSTYRVVSVLTVLHQDWLNGKEY
KCKVSNKALPSSIEKTISKAKGQPREPQVYTLPPCREEMTKNQVSLWCLVKGFYPSDIAVEWESNGQPENNYKTTPP
VLDSDGSFFLYSKLTVDKSRWQQGNVFSCSVMHEALHNRFTQKSLSLSPGK
SEQ ID NO: 16 (3xhmLIGHT single chain used for fusion)
RRSHEVNPAAHLTGANSSLTGSGGPLLWETQLGLAFLRGLSYHDGALVVTKTGYYYIYSKVQLGGVGCPLGLAGTIT
HGLYKRTPRYPEELELLVSQQSPCGRATSSSRVWWDSSFLGGVVHLEAGEKVVVRVLGKRLVRLRDGTRSYFGAFMV
GGGGSGGGGSGGGGSGGGGSRRSHEVNPAAHLTGANSSLTGSGGPLLWETQLGLAFLRGLSYHDGALVVTKTGYYYI
YSKVQLGGVGCPLGLAGTITHGLYKRTPRYPEELELLVSQQSPCGRATSSSRVWWDSSFLGGVVHLEAGEKVVVRVL
GKRLVRLRDGTRSYFGAFMVGGGGSGGGGSGGGGSGGGGSRRSHEVNPAAHLTGANSSLTGSGGPLLWETQLGLAFL
RGLSYHDGALVVTKTGYYYIYSKVQLGGVGCPLGLAGTITHGLYKRTPRYPEELELLVSQQSPCGRATSSSRVWWDS
SFLGGVVHLEAGEKVVVRVLGKRLVRLRDGTRSYFGAFMV
SEQ ID NO: 17 (LTa1b2 used for fusion)
KPAAHLIGDPSKQNSLLWRANTDRAFLQDGFSLSNNSLLVPTSGIYEVYSQVVESGKAYSPKATSSPLYLAHEVQLF
SSQYPFHVPLLSSQKMVYPGLQEPWLHSMYHGAAFQLTQGDQLSTHTDGIPHLVLSPSTVFFGAFALLSPGLPAAHL
IGAPLKGQGLGWETTKEQAFLTSGTQFSDAEGLALPQDGLYYLYCLVGYRGRAPPGGGDPQGRSVTLRSSLYRAGGA
YGPGTPELLLEGAETVTPVLDPARRQGYGPLWYTSVGFGGLVQLRRGERVYVNISHPDMVDFARGKTFFGAVMVGLS
PGLPAAHLIGAPLKGQGLGWETTKEQAFLTSGTQFSDAEGLALPQDGLYYLYCLVGYRGRAPPGGGDPQGRSVTLRS
SLYRAGGAYGPGTPELLLEGAETVTPVLDPARRQGYGPLWYTSVGFGGLVQLRRGERVYVNISHPDMVDFARGKTFF
GAVMVG
SEQ ID NO: 18 (HC B21M fusion to LTa1b2 IgG1s)
QITLKESGPTLVKPTQTLTLTCTFSGFSLSTSGMGVSWIRQPPGKALEWLAHIYWDDDKRYNPSLKSRLTITKDTSK
NQVVLTMTNMDPVDTATYYCARLYGFTYGFAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTHTCPP
CPAPEAAGASSVFLFPPKPKDTLMISRTPEVTCVVVDVSAEDPEVKFNWYVDGVEVHNAKTKPREEQYNSTYRVVSV
LTVLHQDWLNGKEYKCKVSNKALPSSIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLSLSPGKAHSTLKPAAHLI
GDPSKQNSLLWRANTDRAFLQDGFSLSNNSLLVPTSGIYEVYSQVVESGKAYSPKATSSPLYLAHEVQLFSSQYPFH
VPLLSSQKMVYPGLQEPWLHSMYHGAAFQLTQGDQLSTHTDGIPHLVLSPSTVFFGAFALLSPGLPAAHLIGAPLKG
QGLGWETTKEQAFLTSGTQFSDAEGLALPQDGLYYLYCLVGYRGRAPPGGGDPQGRSVTLRSSLYRAGGAYGPGTPE
LLLEGAETVTPVLDPARRQGYGPLWYTSVGFGGLVQLRRGERVYVNISHPDMVDFARGKTFFGAVMVGLSPGLPAAH
LIGAPLKGQGLGWETTKEQAFLTSGTQFSDAEGLALPQDGLYYLYCLVGYRGRAPPGGGDPQGRSVTLRSSLYRAGG
AYGPGTPELLLEGAETVTPVLDPARRQGYGPLWYTSVGFGGLVQLRRGERVYVNISHPDMVD FARGKTFFGAVMVG
SEQ ID NO: 19 (HC B21M fusion to LTa1b2, IgG1s, knob, with pA mutations)
QITLKESGPTLVKPTQTLTLTCTFSGFSLSTSGMGVSWIRQPPGKALEWLAHIYWDDDKRYNPSLKSRLTITKDTSK
NQVVLTMTNMDPVDTATYYCARLYGFTYGFAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTHTCPP
CPAPEAAGASSVFLFPPKPKDTLMISRTPEVTCVVVDVSAEDPEVKFNWYVDGVEVHNAKTKPREEQYNSTYRVVSV
LTVLHQDWLNGKEYKCKVSNKALPSSIEKTISKAKGQPREPQVYTLPPCREEMTKNQVSLWCLVKGFYPSDIAVEWE
SNGQPENNYKTTPPVLDSDGSFFLYSKLTVDKSRWQQGNVFSCSVMHEALHNRFTQKSLSLSPGKAHSTLKPAAHLI
GDPSKQNSLLWRANTDRAFLQDGFSLSNNSLLVPTSGIYEVYSQVVESGKAYSPKATSSPLYLAHEVQLFSSQYPFH
VPLLSSQKMVYPGLQEPWLHSMYHGAAFQLTQGDQLSTHTDGIPHLVLSPSTVFFGAFALLSPGLPAAHLIGAPLKG
QGLGWETTKEQAFLTSGTQFSDAEGLALPQDGLYYLYCLVGYRGRAPPGGGDPQGRSVTLRSSLYRAGGAYGPGTPE
LLLEGAETVTPVLDPARRQGYGPLWYTSVGFGGLVQLRRGERVYVNISHPDMVDFARGKTFFGAVMVGLSPGLPAAH
LIGAPLKGQGLGWETTKEQAFLTSGTQFSDAEGLALPQDGLYYLYCLVGYRGRAPPGGGDPQGRSVTLRSSLYRAGG
AYGPGTPELLLEGAETVTPVLDPARRQGYGPLWYTSVGFGGLVQLRRGERVYVNISHPDMVDFARGKTFFGAVMVG
SEQ ID NO: 20 (HC L19 fusion to LTa1b2 IgG1s)
EVQLLESGGGLVQPGGSLRLSCAASGFTFSSFSMSWVRQAPGKGLEWVSSISGSSGTTYYADSVKGRFTISRDNSKN
TLYLQMNSLRAEDTAVYYCAKPFPYFDYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTHTCPPCPAP
EAAGASSVFLFPPKPKDTLMISRTPEVTCVVVDVSAEDPEVKFNWYVDGVEVHNAKTKPREEQYNSTYRVVSVLTVL
HQDWLNGKEYKCKVSNKALPSSIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGKAHSTLKPAAHLIGDPS
KQNSLLWRANTDRAFLQDGFSLSNNSLLVPTSGIYFVYSQVVFSGKAYSPKATSSPLYLAHEVQLFSSQYPFHVPLL
SSQKMVYPGLQEPWLHSMYHGAAFQLTQGDQLSTHTDGIPHLVLSPSTVFFGAFALLSPGLPAAHLIGAPLKGQGLG
WETTKEQAFLTSGTQFSDAEGLALPQDGLYYLYCLVGYRGRAPPGGGDPQGRSVTLRSSLYRAGGAYGPGTPELLLE
GAETVTPVLDPARRQGYGPLWYTSVGFGGLVQLRRGERVYVNISHPDMVDFARGKTFFGAVMVGLSPGLPAAHLIGA
PLKGQGLGWETTKEQAFLTSGTQFSDAEGLALPQDGLYYLYCLVGYRGRAPPGGGDPQGRSVTLRSSLYRAGGAYGP
GTPELLLEGAETVTPVLDPARRQGYGPLWYTSVGFGGLVQLRRGERVYVNISHPDMVDFARGKTFFGAVMVG
SEQ ID NO: 21 (HC L19 fusion to LTa1b2, IgG1s, knob, with pA mutations)
EVQLLESGGGLVQPGGSLRLSCAASGFTFSSFSMSWVRQAPGKGLEWVSSISGSSGTTYYADSVKGRFTISRDNSKN
TLYLQMNSLRAEDTAVYYCAKPFPYFDYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTHTCPPCPAP
EAAGASSVFLFPPKPKDTLMISRTPEVTCVVVDVSAEDPEVKFNWYVDGVEVHNAKTKPREEQYNSTYRVVSVLTVL
HQDWLNGKEYKCKVSNKALPSSIEKTISKAKGQPREPQVYTLPPCREEMTKNQVSLWCLVKGFYPSDIAVEWESNGQ
PENNYKTTPPVLDSDGSFFLYSKLTVDKSRWQQGNVFSCSVMHEALHNRFTQKSLSLSPGKAHSTLKPAAHLIGDPS
KQNSLLWRANTDRAFLQDGFSLSNNSLLVPTSGIYFVYSQVVFSGKAYSPKATSSPLYLAHEVQLFSSQYPFHVPLL
SSQKMVYPGLQEPWLHSMYHGAAFQLTQGDQLSTHTDGIPHLVLSPSTVFFGAFALLSPGLPAAHLIGAPLKGQGLG
WETTKEQAFLTSGTQFSDAEGLALPQDGLYYLYCLVGYRGRAPPGGGDPQGRSVTLRSSLYRAGGAYGPGTPELLLE
GAETVTPVLDPARRQGYGPLWYTSVGFGGLVQLRRGERVYVNISHPDMVDFARGKTFFGAVMVGLSPGLPAAHLIGA
PLKGQGLGWETTKEQAFLTSGTQFSDAEGLALPQDGLYYLYCLVGYRGRAPPGGGDPQGRSVTLRSSLYRAGGAYGP
GTPELLLEGAETVTPVLDPARRQGYGPLWYTSVGFGGLVQLRRGERVYVNISHPDMVDFARGKTFFGAVMVG
SEQ ID NO: 22 [stapled scFv BHA10 (VH-VL)]
QVQLVQSGAEVKKPGSSVKVSCKASGYTFTTYYLHWVRQAPGCGLEWMGWIYPGNVHAQYNEKFKGRVTITADKSTS
TAYMELSSLRSEDTAVYYCARSWEGFPYWGQGTTVTVSSGGGSGGGSGCPPCGGGGDIQMTQSPSSLSASVGDRVTI
TCKASQNVGINVAWYQQKPGKAPKSLISSASYRYSGVPSRFSGSGSGTDFTLTISSLQPEDFATYFCQQYDTYPFTF
GCGTKVEIK
SEQ ID NO: 23 [stapled scFv BHA10 (VL-VH)]
DIQMTQSPSSLSASVGDRVTITCKASQNVGINVAWYQQKPGCAPKSLISSASYRYSGVPSRFSGSGSGTDFTLTISS
LQPEDFATYFCQQYDTYPFTFGQGTKVEIKGGSGGSGGCPPCGSGGQVQLVQSGAEVKKPGSSVKVSCKASGYTFTT
YYLHWVRQAPGQGLEWMGWIYPGNVHAQYNEKFKGRVTITADKSTSTAYMELSSLRSEDTAVYYCARSWEGFPYWGC
GTTVTVSS
SEQ ID NO: 24 [stapled linker (VH-VL)]
GGGSGGGSGCPPCGGGG
SEQ ID NO: 25 stabilized scFv BHA10 (VH-VL)]
QVQLVQSGAEVKKPGSSVKVSCKASGYTFTTYYLHWVRQAPGQCLEWMGWIYPGNVHAQYNEKFKGRVTITADKSTS
TAYMELSSLRSEDTAVYYCARSWEGFPYWGQGTTVTVSSGGGGSGGGGSGGGGSGGGGSDIQMTQSPSSLSASVGDR
VTITCKASQNVGINVAWYQQKPGKAPKSLISSASYRYSGVPSRFSGSGSGTDFTLTISSLQPEDFATYFCQQYDTYP
FTFGCGTKVEIK
SEQ ID NO: 26 (HC B21M N-term stapled BHA10 (VH-VL), IgG1s, knob, with pA mutations)
QVQLVQSGAEVKKPGSSVKVSCKASGYTFTTYYLHWVRQAPGCGLEWMGWIYPGNVHAQYNEKFKGRVTITADKSTS
TAYMELSSLRSEDTAVYYCARSWEGFPYWGQGTTVTVSSGGGSGGGSGCPPCGGGGDIQMTQSPSSLSASVGDRVTI
TCKASQNVGINVAWYQQKPGKAPKSLISSASYRYSGVPSRFSGSGSGTDFTLTISSLQPEDFATYFCQQYDTYPFTF
GCGTKVEIKGGGGSGGGGSGGGGSGGGGSGGGGSQITLKESGPTLVKPTQTLTLTCTFSGFSLSTSGMGVSWIRQPP
GKALEWLAHIYWDDDKRYNPSLKSRLTITKDTSKNQVVLTMTNMDPVDTATYYCARLYGFTYGFAYWGQGTLVTVSS
ASTKGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPEAAGASSVFLFPPKPKDTLMISRTPEVTCVVVDVSAEDP
EVKFNWYVDGVEVHNAKTKPREEQYNSTYRVVSVLTVLHQDWLNGKEYKCKVSNKALPSSIEKTISKAKGQPREPQV
YTLPPCREEMTKNQVSLWCLVKGFYPSDIAVEWESNGQPENNYKTTPPVLDSDGSFFLYSKLTVDKSRWQQGNVFSC
SVMHEALHNRFTQKSLSLSPGK
SEQ ID NO: 27 (HCB21M N-term stapled BHA10 (VL-VH), IgG1s, knob, with pA mutations)
DIQMTQSPSSLSASVGDRVTITCKASQNVGINVAWYQQKPGCAPKSLISSASYRYSGVPSRFSGSGSGTDFTLTISS
LQPEDFATYFCQQYDTYPFTFGQGTKVEIKGGSGGSGGCPPCGSGGQVQLVQSGAEVKKPGSSVKVSCKASGYTFTT
YYLHWVRQAPGQGLEWMGWIYPGNVHAQYNEKFKGRVTITADKSTSTAYMELSSLRSEDTAVYYCARSWEGFPYWGC
GTTVTVSSGGGGSGGGGSGGGGSGGGGSGGGGSQITLKESGPTLVKPTQTLTLTCTFSGFSLSTSGMGVSWIRQPPG
KALEWLAHIYWDDDKRYNPSLKSRLTITKDTSKNQVVLTMTNMDPVDTATYYCARLYGFTYGFAYWGQGTLVTVSSA
STKGPSVFPLAPSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGASSVFLFPPKPKDTLMISRTPEVTCVVVDVSAEDPE
VKFNWYVDGVEVHNAKTKPREEQYNSTYRVVSVLTVLHQDWLNGKEYKCKVSNKALPSSIEKTISKAKGQPREPQVY
TLPPCREEMTKNQVSLWCLVKGFYPSDIAVEWESNGQPENNYKTTPPVLDSDGSFFLYSKLTVDKSRWQQGNVFSCS
VMHEALHNRFTQKSLSLSPGK
SEQ ID NO: 28 (HC B21MC-term stapled BHA (VH-VL), IgG1s, knob, with pA mutations)
QITLKESGPTLVKPTQTLTLTCTFSGFSLSTSGMGVSWIRQPPGKALEWLAHIYWDDDKRYNPSLKSRLTITKDTSK
NQVVLTMTNMDPVDTATYYCARLYGFTYGFAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTHTCPP
CPAPEAAGASSVFLFPPKPKDTLMISRTPEVTCVVVDVSAEDPEVKFNWYVDGVEVHNAKTKPREEQYNSTYRVVSV
LTVLHQDWLNGKEYKCKVSNKALPSSIEKTISKAKGQPREPQVYTLPPCREEMTKNQVSLWCLVKGFYPSDIAVEWE
SNGQPENNYKTTPPVLDSDGSFFLYSKLTVDKSRWQQGNVFSCSVMHEALHNRFTQKSLSLSPGKGGGGSGGGGSGG
GGSQVQLVQSGAEVKKPGSSVKVSCKASGYTFTTYYLHWVRQAPGCGLEWMGWIYPGNVHAQYNEKFKGRVTITADK
STSTAYMELSSLRSEDTAVYYCARSWEGFPYWGQGTTVTVSSGGGSGGGSGCPPCGGGGDIQMTQSPSSLSASVGDR
VTITCKASQNVGINVAWYQQKPGKAPKSLISSASYRYSGVPSRFSGSGSGTDFTLTISSLQPEDFATYFCQQYDTYP
FTFGCGTKVEIK
SEQ ID NO: 29 (HC B21MC-term stapled BHA (VL-VH), IgG1s, knob, with pA mutations)
QITLKESGPTLVKPTQTLTLTCTFSGFSLSTSGMGVSWIRQPPGKALEWLAHIYWDDDKRYNPSLKSRLTITKDTSK
NQVVLTMTNMDPVDTATYYCARLYGFTYGFAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTHTCPP
CPAPEAAGASSVFLFPPKPKDTLMISRTPEVTCVVVDVSAEDPEVKFNWYVDGVEVHNAKTKPREEQYNSTYRVVSV
LTVLHQDWLNGKEYKCKVSNKALPSSIEKTISKAKGQPREPQVYTLPPCREEMTKNQVSLWCLVKGFYPSDIAVEWE
SNGQPENNYKTTPPVLDSDGSFFLYSKLTVDKSRWQQGNVFSCSVMHEALHNRFTQKSLSLSPGKGGGGSGGGGSGG
GGSDIQMTQSPSSLSASVGDRVTITCKASQNVGINVAWYQQKPGCAPKSLISSASYRYSGVPSRFSGSGSGTDFTLT
ISSLQPEDFATYFCQQYDTYPFTFGQGTKVEIKGGSGGSGGCPPCGSGGQVQLVQSGAEVKKPGSSVKVSCKASGYT
FTTYYLHWVRQAPGQGLEWMGWIYPGNVHAQYNEKFKGRVTITADKSTSTAYMELSSLRSEDTAVYYCARSWEGFPY
WGCGTTVTVSS
SEQ ID NO: 30 (HC L19 N-term stapled BHA10 (VH-VL), IgG1s, knob, with pA mutations)
QVQLVQSGAEVKKPGSSVKVSCKASGYTFTTYYLHWVRQAPGCGLEWMGWIYPGNVHAQYNEKFKGRVTITADKSTS
TAYMELSSLRSEDTAVYYCARSWEGFPYWGQGTTVTVSSGGGSGGGSGCPPCGGGGDIQMTQSPSSLSASVGDRVTI
TCKASQNVGINVAWYQQKPGKAPKSLISSASYRYSGVPSRFSGSGSGTDFTLTISSLQPEDFATYFCQQYDTYPFTF
GCGTKVEIKGGGGSGGGGSGGGGSGGGGSGGGGSEVQLLESGGGLVQPGGSLRLSCAASGETFSSFSMSWVRQAPGK
GLEWVSSISGSSGTTYYADSVKGRFTISRDNSKNTLYLQMNSLRAEDTAVYYCAKPFPYFDYWGQGTLVTVSSASTK
GPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKKVEPKSCDKTHTCPPCPAPEAAGASSVFLFPPKPKDTLMISRTPEVTCVVVDVSAEDPEVKF
NWYVDGVEVHNAKTKPREEQYNSTYRVVSVLTVLHQDWLNGKEYKCKVSNKALPSSIEKTISKAKGQPREPQVYTLP
PCREEMTKNQVSLWCLVKGFYPSDIAVEWESNGQPENNYKTTPPVLDSDGSFFLYSKLTVDKSRWQQGNVFSCSVMH
EALHNRFTQKSLSLSPGK
SEQ ID NO: 31 (HC L19 N-term stapled BHA10 (VL-VH), IgG1s, knob, with pA mutations)
DIQMTQSPSSLSASVGDRVTITCKASQNVGINVAWYQQKPGCAPKSLISSASYRYSGVPSRFSGSGSGTDFTLTISS
LQPEDFATYFCQQYDTYPFTFGQGTKVEIKGGSGGSGGCPPCGSGGQVQLVQSGAEVKKPGSSVKVSCKASGYTFTT
YYLHWVRQAPGQGLEWMGWIYPGNVHAQYNEKFKGRVTITADKSTSTAYMELSSLRSEDTAVYYCARSWEGFPYWGC
GTTVTVSSGGGGSGGGGSGGGGSGGGGSGGGGSEVQLLESGGGLVQPGGSLRLSCAASGFTFSSFSMSWVRQAPGKG
LEWVSSISGSSGTTYYADSVKGRFTISRDNSKNTLYLQMNSLRAEDTAVYYCAKPFPYFDYWGQGTLVTVSSASTKG
PSVFPLAPSSKSTSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPEAAGASSVFLFPPKPKDTLMISRTPEVTCVVVDVSAEDPEVKFN
WYVDGVEVHNAKTKPREEQYNSTYRVVSVLTVLHQDWLNGKEYKCKVSNKALPSSIEKTISKAKGQPREPQVYTLPP
CREEMTKNQVSLWCLVKGFYPSDIAVEWESNGQPENNYKTTPPVLDSDGSFFLYSKLTVDKSRWQQGNVFSCSVMHE
ALHNRFTQKSLSLSPGK
SEQ ID NO: 32 (HC L19C-term stapled BHA10 (VH-VL), IgG1s, knob, with pA mutations)
EVQLLESGGGLVQPGGSLRLSCAASGETFSSFSMSWVRQAPGKGLEWVSSISGSSGTTYYADSVKGRFTISRDNSKN
TLYLQMNSLRAEDTAVYYCAKPFPYFDYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTHTCPPCPAP
EAAGASSVFLFPPKPKDTLMISRTPEVTCVVVDVSAEDPEVKFNWYVDGVEVHNAKTKPREEQYNSTYRVVSVLTVL
HQDWLNGKEYKCKVSNKALPSSIEKTISKAKGQPREPQVYTLPPCREEMTKNQVSLWCLVKGFYPSDIAVEWESNGQ
PENNYKTTPPVLDSDGSFFLYSKLTVDKSRWQQGNVFSCSVMHEALHNRFTQKSLSLSPGKGGGGSGGGGSGGGGSQ
VQLVQSGAEVKKPGSSVKVSCKASGYTFTTYYLHWVRQAPGCGLEWMGWIYPGNVHAQYNEKFKGRVTITADKSTST
AYMELSSLRSEDTAVYYCARSWEGFPYWGQGTTVTVSSGGGSGGGSGCPPCGGGGDIQMTQSPSSLSASVGDRVTIT
CKASQNVGINVAWYQQKPGKAPKSLISSASYRYSGVPSRFSGSGSGTDFTLTISSLQPEDFATYFCQQYDTYPFTFG
CGTKVEIK
SEQ ID NO: 33 (HC L19C-term stapled BHA10 (VL-VH), IgG1s, knob, with pA mutations)
EVQLLESGGGLVQPGGSLRLSCAASGETFSSFSMSWVRQAPGKGLEWVSSISGSSGTTYYADSVKGRFTISRDNSKN
TLYLQMNSLRAEDTAVYYCAKPFPYFDYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTHTCPPCPAP
EAAGASSVFLFPPKPKDTLMISRTPEVTCVVVDVSAEDPEVKFNWYVDGVEVHNAKTKPREEQYNSTYRVVSVLTVL
HQDWLNGKEYKCKVSNKALPSSIEKTISKAKGQPREPQVYTLPPCREEMTKNQVSLWCLVKGFYPSDIAVEWESNGQ
PENNYKTTPPVLDSDGSFFLYSKLTVDKSRWQQGNVFSCSVMHEALHNRFTQKSLSLSPGKGGGGSGGGGSGGGGSD
IQMTQSPSSLSASVGDRVTITCKASQNVGINVAWYQQKPGCAPKSLISSASYRYSGVPSRFSGSGSGTDFTLTISSL
QPEDFATYFCQQYDTYPFTFGQGTKVEIKGGSGGSGGCPPCGSGGQVQLVQSGAEVKKPGSSVKVSCKASGYTFTTY
YLHWVRQAPGQGLEWMGWIYPGNVHAQYNEKFKGRVTITADKSTSTAYMELSSLRSEDTAVYYCARSWEGFPYWGCG
TTVTVSS
SEQ ID NO: 34 (HC L19C-term stapled (VL3 Y36F 549Y F87Y) BHA (VH-VL), IgG1s,
knob, with pA mutations)
EVQLLESGGGLVQPGGSLRLSCAASGETFSSFSMSWVRQAPGKGLEWVSSISGSSGTTYYADSVKGRFTISRDNSKN
TLYLQMNSLRAEDTAVYYCAKPFPYFDYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTHTCPPCPAP
EAAGASSVFLFPPKPKDTLMISRTPEVTCVVVDVSAEDPEVKFNWYVDGVEVHNAKTKPREEQYNSTYRVVSVLTVL
HQDWLNGKEYKCKVSNKALPSSIEKTISKAKGQPREPQVYTLPPCREEMTKNQVSLWCLVKGFYPSDIAVEWESNGQ
PENNYKTTPPVLDSDGSFFLYSKLTVDKSRWQQGNVFSCSVMHEALHNRFTQKSLSLSPGKGGGGSGGGGSGGGGSQ
VQLVQSGAEVKKPGSSVKVSCKASGYTFTTYYLHWVRQAPGCGLEWMGWIYPGNVHAQYNEKFKGRVTITADKSTST
AYMELSSLRSEDTAVYYCARSWEGFPYWGQGTTVTVSSGGGSGGGSGCPPCGGGGDIQMTQSPSSLSASVGDRVTIT
CKASQNVGINVAWFQQKPGKAPKSLIYSASYRYSGVPSRFSGSGSGTDFTLTISSLQPEDFATYYCQQYDTYPFTFG
CGTKVEIK
SEQ ID NO: 35 (HC L19C-term stapled (VHCDR1 Y33A) BHA10 (VH-VL), IgG1s, knob,
with pA mutations)
EVQLLESGGGLVQPGGSLRLSCAASGETFSSFSMSWVRQAPGKGLEWVSSISGSSGTTYYADSVKGRFTISRDNSKN
TLYLQMNSLRAEDTAVYYCAKPFPYFDYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTHTCPPCPAP
EAAGASSVFLFPPKPKDTLMISRTPEVTCVVVDVSAEDPEVKFNWYVDGVEVHNAKTKPREEQYNSTYRVVSVLTVL
HQDWLNGKEYKCKVSNKALPSSIEKTISKAKGQPREPQVYTLPPCREEMTKNQVSLWCLVKGFYPSDIAVEWESNGQ
PENNYKTTPPVLDSDGSFFLYSKLTVDKSRWQQGNVFSCSVMHEALHNRFTQKSLSLSPGKGGGGSGGGGSGGGGSQ
VQLVQSGAEVKKPGSSVKVSCKASGYTFTTYALHWVRQAPGCGLEWMGWIYPGNVHAQYNEKFKGRVTITADKSTST
AYMELSSLRSEDTAVYYCARSWEGFPYWGQGTTVTVSSGGGSGGGSGCPPCGGGGDIQMTQSPSSLSASVGDRVTIT
CKASQNVGINVAWYQQKPGKAPKSLISSASYRYSGVPSRFSGSGSGTDFTLTISSLQPEDFATYFCQQYDTYPFTFG
CGTKVEIK
SEQ ID NO: 36 (HC B21MC-term stapled (VL3 Y36F 549Y F87Y) BHA (VH-VL), IgG1s,
knob, with pA mutations)
QITLKESGPTLVKPTQTLTLTCTFSGFSLSTSGMGVSWIRQPPGKALEWLAHIYWDDDKRYNPSLKSRLTITKDTSK
NQVVLTMTNMDPVDTATYYCARLYGFTYGFAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTHTCPP
CPAPEAAGASSVFLFPPKPKDTLMISRTPEVTCVVVDVSAEDPEVKFNWYVDGVEVHNAKTKPREEQYNSTYRVVSV
LTVLHQDWLNGKEYKCKVSNKALPSSIEKTISKAKGQPREPQVYTLPPCREEMTKNQVSLWCLVKGFYPSDIAVEWE
SNGQPENNYKTTPPVLDSDGSFFLYSKLTVDKSRWQQGNVFSCSVMHEALHNRFTQKSLSLSPGKGGGGSGGGGSGG
GGSQVQLVQSGAEVKKPGSSVKVSCKASGYTFTTYYLHWVRQAPGCGLEWMGWIYPGNVHAQYNEKFKGRVTITADK
STSTAYMELSSLRSEDTAVYYCARSWEGFPYWGQGTTVTVSSGGGSGGGSGCPPCGGGGDIQMTQSPSSLSASVGDR
VTITCKASQNVGINVAWFQQKPGKAPKSLIYSASYRYSGVPSRFSGSGSGTDFTLTISSLQPEDFATYYCQQYDTYP
FTFGCGTKVEIK
SEQ ID NO: 37 (HC B21MC-term stapled (VHCDR1 Y33A) BHA10 (VH-VL), IgG1s, knob,
with pA mutations)
QITLKESGPTLVKPTQTLTLTCTFSGFSLSTSGMGVSWIRQPPGKALEWLAHIYWDDDKRYNPSLKSRLTITKDTSK
NQVVLTMTNMDPVDTATYYCARLYGFTYGFAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTHTCPP
CPAPEAAGASSVFLFPPKPKDTLMISRTPEVTCVVVDVSAEDPEVKFNWYVDGVEVHNAKTKPREEQYNSTYRVVSV
LTVLHQDWLNGKEYKCKVSNKALPSSIEKTISKAKGQPREPQVYTLPPCREEMTKNQVSLWCLVKGFYPSDIAVEWE
SNGQPENNYKTTPPVLDSDGSFFLYSKLTVDKSRWQQGNVFSCSVMHEALHNRFTQKSLSLSPGKGGGGSGGGGSGG
GGSQVQLVQSGAEVKKPGSSVKVSCKASGYTFTTYALHWVRQAPGCGLEWMGWIYPGNVHAQYNEKFKGRVTITADK
STSTAYMELSSLRSEDTAVYYCARSWEGFPYWGQGTTVTVSSGGGSGGGSGCPPCGGGGDIQMTQSPSSLSASVGDR
VTITCKASQNVGINVAWYQQKPGKAPKSLISSASYRYSGVPSRFSGSGSGTDFTLTISSLQPEDFATYFCQQYDTYP
FTFGCGTKVEIK
SEQ ID NO: 38 (HC L19C-term stapled BHA10 (VH-VL), IgG1s, knob, no pA mutations)
EVQLLESGGGLVQPGGSLRLSCAASGETFSSFSMSWVRQAPGKGLEWVSSISGSSGTTYYADSVKGRFTISRDNSKN
TLYLQMNSLRAEDTAVYYCAKPFPYFDYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTHTCPPCPAP
EAAGASSVFLFPPKPKDTLMISRTPEVTCVVVDVSAEDPEVKFNWYVDGVEVHNAKTKPREEQYNSTYRVVSVLTVL
HQDWLNGKEYKCKVSNKALPSSIEKTISKAKGQPREPQVYTLPPCREEMTKNQVSLWCLVKGFYPSDIAVEWESNGQ
PENNYKTTPPVLDSDGSFFLYSKLTVDKSRWQQGNVFSCSVMHEALHNHYTQKSLSLSPGKGGGGSGGGGSGGGGSQ
VQLVQSGAEVKKPGSSVKVSCKASGYTFTTYYLHWVRQAPGCGLEWMGWIYPGNVHAQYNEKFKGRVTITADKSTST
AYMELSSLRSEDTAVYYCARSWEGFPYWGQGTTVTVSSGGGSGGGSGCPPCGGGGDIQMTQSPSSLSASVGDRVTIT
CKASQNVGINVAWYQQKPGKAPKSLISSASYRYSGVPSRFSGSGSGTDFTLTISSLQPEDFATYFCQQYDTYPFTFG
CGTKVEIK
SEQ ID NO: 39 (HC L19C-term disulfide stab, BHA (VH-VL), IgG1s, knob,
without pA mutations)
EVQLLESGGGLVQPGGSLRLSCAASGETFSSFSMSWVRQAPGKGLEWVSSISGSSGTTYYADSVKGRFTISRDNSKN
TLYLQMNSLRAEDTAVYYCAKPFPYFDYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTHTCPPCPAP
EAAGASSVFLFPPKPKDTLMISRTPEVTCVVVDVSAEDPEVKFNWYVDGVEVHNAKTKPREEQYNSTYRVVSVLTVL
HQDWLNGKEYKCKVSNKALPSSIEKTISKAKGQPREPQVYTLPPCREEMTKNQVSLWCLVKGFYPSDIAVEWESNGQ
PENNYKTTPPVLDSDGSFFLYSKLTVDKSRWQQGNVFSCSVMHEALHNHYTQKSLSLSPGKGGGGSGGGGSGGGGSQ
VQLVQSGAEVKKPGSSVKVSCKASGYTFTTYYLHWVRQAPGQCLEWMGWIYPGNVHAQYNEKFKGRVTITADKSTST
AYMELSSLRSEDTAVYYCARSWEGFPYWGQGTTVTVSSGGGGSGGGGSGGGGSGGGGSDIQMTQSPSSLSASVGDRV
TITCKASQNVGINVAWYQQKPGKAPKSLISSASYRYSGVPSRFSGSGSGTDFTLTISSLQPEDFATYFCQQYDTYPF
TFGCGTKVEIK
SEQ ID NO: 40 (HC B21MC-term disulfide stab, BHA10 (VH-VL), IgG1s, knob,
without pA mutations)
QITLKESGPTLVKPTQTLTLTCTFSGFSLSTSGMGVSWIRQPPGKALEWLAHIYWDDDKRYNPSLKSRLTITKDTSK
NQVVLTMTNMDPVDTATYYCARLYGFTYGFAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTHTCPP
CPAPEAAGASSVFLFPPKPKDTLMISRTPEVTCVVVDVSAEDPEVKFNWYVDGVEVHNAKTKPREEQYNSTYRVVSV
LTVLHQDWLNGKEYKCKVSNKALPSSIEKTISKAKGQPREPQVYTLPPCREEMTKNQVSLWCLVKGFYPSDIAVEWE
SNGQPENNYKTTPPVLDSDGSFFLYSKLTVDKSRWQQGNVFSCSVMHEALHNHYTQKSLSLSPGKGGGGSGGGGSGG
GGSQVQLVQSGAEVKKPGSSVKVSCKASGYTFTTYYLHWVRQAPGQCLEWMGWIYPGNVHAQYNEKFKGRVTITADK
STSTAYMELSSLRSEDTAVYYCARSWEGFPYWGQGTTVTVSSGGGGSGGGGSGGGGSGGGGSDIQMTQSPSSLSASV
GDRVTITCKASQNVGINVAWYQQKPGKAPKSLISSASYRYSGVPSRFSGSGSGTDFTLTISSLQPEDFATYFCQQYD
TYPFTFGCGTKVEIK
SEQ ID NO: 41 (HC B21MC-term stapled BHA10 (VH-VL), IgG1s, knob, no pA mutations)
QITLKESGPTLVKPTQTLTLTCTFSGFSLSTSGMGVSWIRQPPGKALEWLAHIYWDDDKRYNPSLKSRLTITKDTSK
NQVVLTMTNMDPVDTATYYCARLYGFTYGFAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTHTCPP
CPAPEAAGASSVFLFPPKPKDTLMISRTPEVTCVVVDVSAEDPEVKFNWYVDGVEVHNAKTKPREEQYNSTYRVVSV
LTVLHQDWLNGKEYKCKVSNKALPSSIEKTISKAKGQPREPQVYTLPPCREEMTKNQVSLWCLVKGFYPSDIAVEWE
SNGQPENNYKTTPPVLDSDGSFFLYSKLTVDKSRWQQGNVFSCSVMHEALHNHYTQKSLSLSPGKGGGGSGGGGSGG
GGSQVQLVQSGAEVKKPGSSVKVSCKASGYTFTTYYLHWVRQAPGCGLEWMGWIYPGNVHAQYNEKFKGRVTITADK
STSTAYMELSSLRSEDTAVYYCARSWEGFPYWGQGTTVTVSSGGGSGGGSGCPPCGGGGDIQMTQSPSSLSASVGDR
VTITCKASQNVGINVAWYQQKPGKAPKSLISSASYRYSGVPSRFSGSGSGTDFTLTISSLQPEDFATYFCQQYDTYP
FTFGCGTKVEIK
SEQ ID NO: 42 [(GGGGS)4 linker (used to connect FITfragments in disulfide
stabilized scFv)]
GGGGSGGGGSGGGGSGGGGS
SEQ ID NO: 43 (VH BHA10)
QVQLVQSGAEVKKPGSSVKVSCKASGYTFTTYYLHWVRQAPGQGLEWMGWIYPGNVHAQYNEKFKGRVTITADKSTS
TAYMELSSLRSEDTAVYYCARSWEGFPYWGQGTTVTVSS
SEQ ID NO: 44 (VL BHA10)
DIQMTQSPSSLSASVGDRVTITCKASQNVGINVAWYQQKPGKAPKSLISSASYRYSGVPSRFSGSGSGTDFTLTISS
LQPEDFATYFCQQYDTYPFTFGQGTKVEIK
SEQ ID NO: 45 (VH L19)
EVQLLESGGGLVQPGGSLRLSCAASGETFSSFSMSWVRQAPGKGLEWVSSISGSSGTTYYADSVKGRFTISRDNSKN
TLYLQMNSLRAEDTAVYYCAKPFPYFDYWGQGTLVTVSS
SEQ ID NO: 46 (VL L19)
EIVLTQSPGTLSLSPGERATLSCRASQSVSSSFLAWYQQKPGQAPRLLIYYASSRATGIPDRFSGSGSGTDFTLTIS
RLEPEDFAVYYCQQTGRIPPTFGQGTKVEIK
SEQ ID NO: 47 (VHCBE11)
EVQLVESGGGLVKPGGSLRLSCAASGETFSDYYMYWFRQAPGKGLEWVATISDGGSYTYYPDSVKGRFTISRDNAKN
SLYLQMSSLRAEDTAVYYCAREENGNFYYFDYWGQGTTVTVSS
SEQ ID NO: 48 (VLCBE11)
DIQMTQSPSSLSASVGDRVTITCKAGQDIKSYLSWYQQKPGKAPKLLIYYATRLADGVPSRFSGSGSGTDYTLTISS
LQPEDFATYYCLQHGESPWTFGGGTKLEIK
SEQ ID NO: 49 (VH B21M)
QITLKESGPTLVKPTQTLTLTCTFSGESLSTSGMGVSWIRQPPGKALEWLAHIYWDDDKRYNPSLKSRLTITKDTSK
NQVVLTMTNMDPVDTATYYCARLYGFTYGFAYWGQGTLVTVSS
SEQ ID NO: 50 (VL B21M)
DIVMTQSPDSLAVSLGERATINCRASQSVDYNGISYMHWYQQKPGQPPKLLIYAASNPESGVPDRFSGSGSGTDFTL
TISSLQAEDVAVYYCQQIIEDPWTFGQGTKVEIK
SEQ ID NO: 51 (Fibronectin domains 7B89)
PLSPPTNLHLEANPDTGVLTVSWERSTTPDITGYRITTTPTNGQQGNSLEEVVHADQSSCTEDNLSPGLEYNVSVYT
VKDDKESVPISDTIIPEVPQLTDLSFVDITDSSIGLRWTPLNSSTIIGYRITVVAAGEGIPIFEDFVDSSVGYYTVT
GLEPGIDYDISVITLINGGESAPTTLTQQTAVPPPTDLRFTNIGPDTMRVTWAPPPSIDLTNELVRYSPVKNEEDVA
ELSISPSDNAVVLTNLLPGTEYVVSVSSVYEQHESTPLRGRQKTGLDSPTGIDESDITANSFTVHWIAPRATITGYR
IRHHPEHFSGRPREDRVPHSRNSITLTNLTPGTEYVVSIVALNGREESPLLIGQQSTHHHHHH
SEQ ID NO: 52 (Fibronectin domains 789)
PLSPPTNLHLEANPDTGVLTVSWERSTTPDITGYRITTTPTNGQQGNSLEEVVHADQSSCTEDNLSPGLEYNVSVYT
VKDDKESVPISDTIIPAVPPPTDLRFTNIGPDTMRVTWAPPPSIDLTNELVRYSPVKNEEDVAELSISPSDNAVVLT
NLLPGTEYVVSVSSVYEQHESTPLRGRQKTGLDSPTGIDESDITANSFTVHWIAPRATITGYRIRHHPEHFSGRPRE
DRVPHSRNSITLTNLTPGTEYVVSIVALNGREESPLLIGQQSTHHHHHH
SEQ ID NO: 53 [stapled scFv (VL3 Y36F 549Y F87Y) BHA10 (VH-VL)]
QVQLVQSGAEVKKPGSSVKVSCKASGYTETTYYLHWVRQAPGCGLEWMGWIYPGNVHAQYNEKFKGRVTITADKSTS
TAYMELSSLRSEDTAVYYCARSWEGFPYWGQGTTVTVSSGGGSGGGSGCPPCGGGGDIQMTQSPSSLSASVGDRVTI
TCKASQNVGINVAWFQQKPGKAPKSLIYSASYRYSGVPSRFSGSGSGTDFTLTISSLQPEDFATYYCQQYDTYPFTF
GCGTKVEIK
SEQ ID NO: 54 [stapled scFv (VHCDR1 Y33A) BHA10 (VH-VL)]
QVQLVQSGAEVKKPGSSVKVSCKASGYTETTYALHWVRQAPGCGLEWMGWIYPGNVHAQYNEKFKGRVTITADKSTS
TAYMELSSLRSEDTAVYYCARSWEGFPYWGQGTTVTVSSGGGSGGGSGCPPCGGGGDIQMTQSPSSLSASVGDRVTI
TCKASQNVGINVAWYQQKPGKAPKSLISSASYRYSGVPSRFSGSGSGTDFTLTISSLQPEDFATYFCQQYDTYPFTF
GCGTKVEIK
SEQ ID NO: 55 [stapled linker (VL-VH)]
GGSGGSGGCPPCGSGG
SEQ ID NO: 56 (HC L19C-term disulfide stab BHA10 (VH-VL), IgG1s, knob, with pA
mutations)
EVQLLESGGGLVQPGGSLRLSCAASGFTESSFSMSWVRQAPGKGLEWVSSISGSSGTTYYADSVKGRETISRDNSKN
TLYLQMNSLRAEDTAVYYCAKPFPYFDYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTHTCPPCPAP
EAAGASSVFLEPPKPKDTLMISRTPEVTCVVVDVSAEDPEVKFNWYVDGVEVHNAKTKPREEQYNSTYRVVSVLTVL
HQDWLNGKEYKCKVSNKALPSSIEKTISKAKGQPREPQVYTLPPCREEMTKNQVSLWCLVKGFYPSDIAVEWESNGQ
PENNYKTTPPVLDSDGSFELYSKLTVDKSRWQQGNVESCSVMHEALHNRFTQKSLSLSPGKGGGGSGGGGSGGGGSQ
VQLVQSGAEVKKPGSSVKVSCKASGYTETTYYLHWVRQAPGQCLEWMGWIYPGNVHAQYNEKFKGRVTITADKSTST
AYMELSSLRSEDTAVYYCARSWEGFPYWGQGTTVTVSSGGGGSGGGGSGGGGSGGGGSDIQMTQSPSSLSASVGDRV
TITCKASQNVGINVAWYQQKPGKAPKSLISSASYRYSGVPSRFSGSGSGTDFTLTISSLQPEDFATYFCQQYDTYPF
TFGCGTKVEIK
SEQ ID NO: 57 (HC B21MC-term disulfide stab, BHA10 (VH-VL), IgG1s, knob,
with pA mutations)
QITLKESGPTLVKPTQTLTLTCTFSGFSLSTSGMGVSWIRQPPGKALEWLAHIYWDDDKRYNPSLKSRLTITKDTSK
NQVVLTMTNMDPVDTATYYCARLYGFTYGFAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTHTCPP
CPAPEAAGASSVFLFPPKPKDTLMISRTPEVTCVVVDVSAEDPEVKFNWYVDGVEVHNAKTKPREEQYNSTYRVVSV
LTVLHQDWLNGKEYKCKVSNKALPSSIEKTISKAKGQPREPQVYTLPPCREEMTKNQVSLWCLVKGFYPSDIAVEWE
SNGQPENNYKTTPPVLDSDGSFFLYSKLTVDKSRWQQGNVFSCSVMHEALHNRFTQKSLSLSPGKGGGGSGGGGSGG
GGSQVQLVQSGAEVKKPGSSVKVSCKASGYTFTTYYLHWVRQAPGQCLEWMGWIYPGNVHAQYNEKFKGRVTITADK
STSTAYMELSSLRSEDTAVYYCARSWEGFPYWGQGTTVTVSSGGGGSGGGGSGGGGSGGGGSDIQMTQSPSSLSASV
GDRVTITCKASQNVGINVAWYQQKPGKAPKSLISSASYRYSGVPSRFSGSGSGTDFTLTISSLQPEDFATYFCQQYD
TYPFTFGCGTKVEIK
SEQ ID NO: 58 (IgG1 sigma Fc)
DKTHTCPPCPAPEAAGASSVFLFPPKPKDTLMISRTPEVTCVVVDVSAEDPEVKFNWYVDGVEVHNAKTKPREEQYN
STYRVVSVLTVLHQDWLNGKEYKCKVSNKALPSSIEKTISKAKGQPREPQVYTLPPSREEMTKNQVSLTCLVKGFYP
SDIAVEWESNGQPENNYKTTPPVLDSDGSFFLYSKLTVDKSRWQQGNVFSCSVMHEALHNHYTQKSLSLSPGK
SEQ ID NO: 59 [(GGGGS)3 linker scFv to Fc]
GGGGSGGGGSGGGGS
SEQ ID NO: 60 (HCDR1 of VH BHA10)
TYYLH
SEQ ID NO: 61 (HCDR2 of VH BHA10)
WIYPGNVHAQYNEKFKG
SEQ ID NO: 62 (HCDR3 of VH BHA10)
SWEGFPY
SEQ ID NO: 63 (LCDR1 of VL BHA10)
KASQNVGINVA
SEQ ID NO: 64 (LCDR2 of VL BHA10)
SASYRYS
SEQ ID NO: 65 (LCDR3 of VL BHA10)
QQYDTYPFT
SEQ ID NO: 66 (HCDR1 of VHCBE11)
DYYMY
SEQ ID NO: 67 (HCDR2 of VHCBE11)
TISDGGSYTYYPDSVK
SEQ ID NO: 68 (HCDR3 of VHCBE11)
EENGNFYYFDY
SEQ ID NO: 69 (LCDR1 of VLCBE11)
KAGQDIKSYLS
SEQ ID NO: 70 (LCDR2 of VLCBE11)
YATRLAD
SEQ ID NO: 71 (LCDR3 of VLCBE11)
LQHGESPWT
SEQ ID NO: 72 (HCDR1 of VH L19)
SFSMS
SEQ ID NO: 73 (HCDR2 of VH L19)
SISGSSGTTYYADSVKG
SEQ ID NO: 74 (HCDR3 of VH L19)
PFPYFDY
SEQ ID NO: 75 (LCDR1 of VL L19)
RASQSVSSSFLA
SEQ ID NO: 76 (LCDR2 of VL L19)
YASSPAT
SEQ ID NO: 77 (LCDR3 of VL L19)
QQTGRIPPT
SEQ ID NO: 78 (VH MSLNmAb1)
QVQLQQSGPELEKPGASVKISCKASGYSFTGYTMNWVKQSHGKSLEWIGLITPYNGASSYNQKFRGKATLTVDKSSS
TAYMDLLSLTSEDSAVYFCARGGYDGRGFDYWGSGTPVTVSS
SEQ ID NO: 79 (VL MSLNmAb1)
DIELTQSPAIMSASPGEKVTMTCSASSSVSYMHWYQQKSGTSPKRWIYDTSKLASGVPGRFSGSGSGNSYSLTISSV
EAEDDATYYCQQWSKHPLTFGSGTKVEIK
SEQ ID NO: 80 (MSLNmAb1 HCC-term stapled BHA10 (VH-VL), IgG1s, knob, with pA
mutations)
QVQLQQSGPELEKPGASVKISCKASGYSFTGYTMNWVKQSHGKSLEWIGLITPYNGASSYNQKFRGKATLTVDKSSS
TAYMDLLSLTSEDSAVYFCARGGYDGRGFDYWGSGTPVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVDKKVEPKSCDKTHTCPPC
PAPEAAGASSVFLEPPKPKDTLMISRTPEVTCVVVDVSAEDPEVKFNWYVDGVEVHNAKTKPREEQYNSTYRVVSVL
TVLHQDWLNGKEYKCKVSNKALPSSIEKTISKAKGQPREPQVYTLPPCREEMTKNQVSLWCLVKGFYPSDIAVEWES
NGQPENNYKTTPPVLDSDGSFELYSKLTVDKSRWQQGNVESCSVMHEALHNRFTQKSLSLSPGKGGGGSGGGGSGGG
GSQVQLVQSGAEVKKPGSSVKVSCKASGYTETTYYLHWVRQAPGCGLEWMGWIYPGNVHAQYNEKFKGRVTITADKS
TSTAYMELSSLRSEDTAVYYCARSWEGFPYWGQGTTVTVSSGGGSGGGSGCPPCGGGGDIQMTQSPSSLSASVGDRV
TITCKASQNVGINVAWYQQKPGKAPKSLISSASYRYSGVPSRFSGSGSGTDFTLTISSLQPEDFATYFCQQYDTYPF
TFGCGTKVEIK
SEQ ID NO: 81 (HC MSLNmAb1 IgG1s hole)
QVQLQQSGPELEKPGASVKISCKASGYSFTGYTMNWVKQSHGKSLEWIGLITPYNGASSYNQKFRGKATLTVDKSSS
TAYMDLLSLTSEDSAVYFCARGGYDGRGFDYWGSGTPVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVDKKVEPKSCDKTHTCPPC
PAPEAAGASSVFLEPPKPKDTLMISRTPEVTCVVVDVSAEDPEVKFNWYVDGVEVHNAKTKPREEQYNSTYRVVSVL
TVLHQDWLNGKEYKCKVSNKALPSSIEKTISKAKGQPREPQVCTLPPSREEMTKNQVSLSCAVKGFYPSDIAVEWES
NGQPENNYKTTPPVLDSDGSFELVSKLTVDKSRWQQGNVESCSVMHEALHNHYTQKSLSLSPGK
SEQ ID NO: 82 (LC MLSNmAb1)
DIELTQSPAIMSASPGEKVTMTCSASSSVSYMHWYQQKSGTSPKRWIYDTSKLASGVPGRESGSGSGNSYSLTISSV
EAEDDATYYCQQWSKHPLTFGSGTKVEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC
SEQ ID NO: 83 (BHA10 HCDR1 low affinity variant)
TYALH
SEQ ID NO: 84 (HC L19 IgG1s knob with pA mutations)
VQLLESGGGLVQPGGSLRLSCAASGETFSSFSMSWVRQAPGKGLEWVSSISGSSGTTYYADSVKGRFTISRDNSKNT
LYLQMNSLRAEDTAVYYCAKPFPYFDYWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKVDKKVEPKSCDKTHTCPPCPAPE
AAGASSVFLFPPKPKDTLMISRTPEVTCVVVDVSAEDPEVKFNWYVDGVEVHNAKTKPREEQYNSTYRVVSVLTVLH
QDWLNGKEYKCKVSNKALPSSIEKTISKAKGQPREPQVYTLPPCREEMTKNQVSLWCLVKGFYPSDIAVEWESNGQP
ENNYKTTPPVLDSDGSFFLYSKLTVDKSRWQQGNVFSCSVMHEALHNRFTQKSLSLSPGK
SEQ ID NO: 85 (HC B21M (RSV) IgG1s knob with pA mutations)
QITLKESGPTLVKPTQTLTLTCTFSGFSLSTSGMGVSWIRQPPGKALEWLAHIYWDDDKRYNPSLKSRLTITKDTSK
NQVVLTMTNMDPVDTATYYCARLYGFTYGFAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTHTCPP
CPAPEAAGASSVFLFPPKPKDTLMISRTPEVTCVVVDVSAEDPEVKFNWYVDGVEVHNAKTKPREEQYNSTYRVVSV
LTVLHQDWLNGKEYKCKVSNKALPSSIEKTISKAKGQPREPQVYTLPPCREEMTKNQVSLWCLVKGFYPSDIAVEWE
SNGQPENNYKTTPPVLDSDGS FFLYSKLTVDKSRWQQGNVFSCSVMHEALHNRFTQKSLSLSPGK
SEQ ID NO: 86 (MSLNmAbl HC, IgG1s, knob, with pA mutations)
QVQLQQSGPELEKPGASVKISCKASGYSFTGYTMNWVKQSHGKSLEWIGLITPYNGASSYNQKFRGKATLTVDKSSS
TAYMDLLSLTSEDSAVYFCARGGYDGRGFDYWGSGTPVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVDKKVEPKSCDKTHTCPPC
PAPEAAGASSVFLFPPKPKDTLMISRTPEVTCVVVDVSAEDPEVKFNWYVDGVEVHNAKTKPREEQYNSTYRVVSVL
TVLHQDWLNGKEYKCKVSNKALPSSIEKTISKAKGQPREPQVYTLPPCREEMTKNQVSLWCLVKGFYPSDIAVEWES
NGQPENNYKTTPPVLDSDGSFFLYSKLTVDKSRWQQGNVFSCSVMHEALHNRFTQKSLSLSPGK

1. A multispecific binding molecule comprising:
(iii) a first binding domain that specifically binds to a lymphotoxin beta receptor (LTBR), and
(iv) a second binding domain that specifically binds to extra domain B (EDB) of fibronectin,
wherein the multispecific binding molecule activates LTBR upon binding of the EDB.

2. The multispecific binding molecule of claim 1, wherein the multispecific binding molecule activates LTBR in a tumor specific manner.

3. The multispecific binding molecule of claim 1, wherein the multispecific binding molecule is a bispecific antibody.

4. The multispecific binding molecule of claim 1, wherein the multispecific binding molecule comprises two antigen binding domains.

5. The multispecific binding molecule of claim 1, wherein the multispecific binding molecule comprises three antigen binding domains.

6. The multispecific binding molecule of claim 5, wherein the three antigen binding domains comprise one binding domain that specifically binds to LTBR.

7. The multispecific binding molecule of claim 5, wherein the three antigen binding domains comprise two binding domains that specifically bind EDB.

8. The multispecific binding molecule of claim 5, wherein the binding domain that specifically binds to LTBR comprises a single chain variable domain of an antibody.

9. The multispecific binding molecule of claim 1, wherein the first binding domain that specifically binds to LTBR comprises amino acid sequences selected from the group consisting of:
(i) HCDR1, HCDR2 and HCDR3 comprising the amino acid sequences of SEQ ID NO:60, SEQ ID NO:61, and SEQ ID NO:62, respectively, and LCDR1, LCDR2 and LCDR3 comprising the amino acid sequences of SEQ ID NO:63, SEQ ID NO:64, and SEQ ID NO:65, respectively;
(ii) HCDR1, HCDR2 and HCDR3 comprising the amino acid sequences of SEQ ID NO:83, SEQ ID NO:61, and SEQ ID NO:62, respectively, and LCDR1, LCDR2 and LCDR3 comprising the amino acid sequences of SEQ ID NO:63, SEQ ID NO:64, and SEQ ID NO:65, respectively; (iii) HCDR1, HCDR2 and HCDR3 comprising the amino acid sequences of SEQ ID NO:66, SEQ ID NO:67, and SEQ ID NO:68, respectively, and LCDR1, LCDR2, and LCDR3 comprising the amino acid sequences of SEQ ID NO:69, SEQ ID NO:70, and SEQ ID NO:71, respectively;
(iv) VH comprises an amino acid sequence having at least 95%, 96%, 97%, 98%, 99% identity or 100% identity to the amino acid sequence of SEQ ID NO:43, and VL comprises an amino acid sequence having at least 95% identity to the amino acid sequence of SEQ ID NO:44;
(v) VH comprises an amino acid sequence having at least 95% identity to the amino acid sequence of SEQ ID NO:47, and VL comprises an amino acid sequence having at least 95% identity to the amino acid sequence of SEQ ID NO:48:
(vi) SEQ ID NO: 22;
(vii) SEQ ID NO: 23; and
(viii) SEQ ID NO: 25.

10. The multispecific binding molecule of claim 1, wherein the second binding domain that specifically binds to EDB comprises amino acid sequences selected from the group consisting of:
(i) HCDR1, HCDR2 and HCDR3 comprising the amino acid sequences of SEQ ID NO:72, SEQ ID NO:73, and SEQ ID NO:74, respectively, and LCDR1, LCDR2 and LCDR3 comprising the amino acid sequences of SEQ ID NO:75, SEQ ID NO:76, and SEQ ID NO:77, respectively; and
(ii) VH comprises an amino acid sequence having at least 95% identity to the amino acid sequence of SEQ ID NO:45, and VL comprises an amino acid sequence having at least 95% identity to the amino acid sequence of SEQ ID NO:46.

11. The multispecific binding molecule of claim 1, comprising amino acid sequences selected from the group consisting of:
(a) (i) a first heavy chain comprising the amino acid sequence of SEQ ID NO: 1 forming a binding domain with a first light chain comprising the amino acid sequence of SEQ ID NO: 2, and
(ii) a second heavy chain comprising the amino acid sequence of SEQ ID NO: 4 forming a binding domain with a second light chain comprising the amino acid sequence of SEQ ID NO: 5; and
(b) (i) a first heavy chain comprising the amino acid sequence of SEQ ID NO: 9 forming a binding domain with a first light chain comprising the amino acid sequence of SEQ ID NO: 10, and (ii) a second heavy chain comprising the amino acid sequence of SEQ ID NO: 4 forming a binding domain with a second light chain comprising the amino acid sequence of SEQ ID NO: 5.

12. The multispecific binding molecule of claim 1, comprising amino acid sequences selected from the group consisting of:
(a) (i) an scFv-heavy chain fusion comprising the amino acid sequence of SEQ ID NO: 30, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5;
(b) (i) an scFv-heavy chain fusion comprising the amino acid sequence of SEQ ID NO: 31, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5;
(c) (i) an scFv-heavy chain fusion comprising the amino acid sequence of SEQ ID NO: 32, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5;
(d) (i) an scFv-heavy chain fusion comprising the amino acid sequence of SEQ ID NO: 33, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5;
(e) (i) an scFv-heavy chain fusion comprising the amino acid sequence of SEQ ID NO: 34, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5;
(f) (i) an scFv-heavy chain fusion comprising the amino acid sequence of SEQ ID NO: 35, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5;
(g) (i) an scFv-heavy chain fusion comprising the amino acid sequence of SEQ ID NO: 38, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5;
(h) (i) an scFv-heavy chain fusion comprising the amino acid sequence of SEQ ID NO: 39, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5; and
(i) (i) an scFv-heavy chain fusion comprising the amino acid sequence of SEQ ID NO: 56, the heavy chain part thereof forming a binding domain with a light chain comprising the amino acid sequence of SEQ ID NO: 5, and (ii) a heavy chain comprising the amino acid sequence of SEQ ID NO: 4 forming a binding domain with a light chain comprising the amino acid sequence of SEQ ID NO: 5.

13. One or more nucleic acid molecules encoding the multispecific binding molecule of claim 12.

14. One or more vectors comprising the one or more nucleic acid molecules of claim 13.

15. An isolated host cell comprising the one or more nucleic acid molecules of claim 13 or the one or more vectors of claim 14.

16. A pharmaceutical composition comprising the multispecific binding molecule of claim 1 and a pharmaceutically acceptable carrier.

17. A method of treating cancer in a subject in need thereof, comprising administering to the subject the multispecific binding molecule of claim 1.

18. The method of claim 17, further comprising administering a second therapeutic agent selected from a chemotherapy agent, an anti-CD20 mAb, an anti-TIM-3 mAb, an anti-CTLA-4 antibody, an anti-PD-L1 antibody, an anti-PD-1 antibody, a PD-1/PD-L1 therapy, Indoleamine-pyrrole 2,3-dioxygenase (IDO), an anti-OX40 antibody, an anti-GITR antibody, an anti-CD40 antibody, an anti-CD38 antibody, a cytokine, an oncolytic virus, a TLR agonist, a STING agonist, and combinations thereof.
